SEQUENCE LISTING (66 sequences)

SEQ ID NO: 1 - protein, 142 aa, Homo sapiens
SGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKTSATVGPKAPSGGKKATQASQEY

SEQ ID NO: 2 - protein, 4 aa, Artificial
  SQEY motif
SQEY

SEQ ID NO: 3 - protein, 1483 aa, Homo sapiens
MAPLLGRKPFPLVKPLPGEEPLFTIPHTQEAFRTREEYEARLERYSERIWTCKSTGSSQLTHKEAWEEEQEVAELLKEEFPAWYEKLVLEMVHHNTASLEKLVDTAWLEIMTKYAVGEECDFEVGKEKMLKVKIVKIHPLEKVDEEATEKKSDGACDSPSSDKENSSQIAQDHQKKETVVKEDEGRRESINDRARRSPRKLPTSLKKGERKWAPPKFLPHKYDVKLQNEDKIISNVPADSLIRTERPPNKEIVRYFIRHNALRAGTGENAPWVVEDELVKKYSLPSKFSDFLLDPYKYMTLNPSTKRKNTGSPDRKPSKKSKTDNSSLSSPLNPKLWCHVHLKKSLSGSPLKVKNSKNSKSPEEHLEEMMKMMSPNKLHTNFHIPKKGPPAKKPGKHSDKPLKAKGRSKGILNGQKSTGNSKSPKKGLKTPKTKMKQMTLLDMAKGTQKMTRAPRNSGGTPRTSSKPHKHLPPAALHLIAYYKENKDREDKRSALSCVISKTARLLSSEDRARLPEELRSLVQKRYELLEHKKRWASMSEEQRKEYLKKKREELKKKLKEKAKERREKEMLERLEKQKRYEDQELTGKNLPAFRLVDTPEGLPNTLFGDVAMVVEFLSCYSGLLLPDAQYPITAVSLMEALSADKGGFLYLNRVLVILLQTLLQDEIAEDYGELGMKLSEIPLTLHSVSELVRLCLRRSDVQEESEGSDTDDNKDSAAFEDNEVQDEFLEKLETSEFFELTSEEKLQILTALCHRILMTYSVQDHMETRQQMSAELWKERLAVLKEENDKKRAEKQKRKEMEAKNKENGKVENGLGKTDRKKEIVKFEPQVDTEAEDMISAVKSRRLLAIQAKKEREIQEREMKVKLERQAEEERIRKHKAAAEKAFQEGIAKAKLVMRRTPIGTDRNHNRYWLFSDEVPGLFIEKGWVHDSIDYRFNHHCKDHTVSGDEDYCPRSKKANLGKNASMNTQHGTATEVAVETTTPKQGQNLWFLCDSQKELDELLNCLHPQGIRESQLKERLEKRYQDIIHSIHLARKPNLGLKSCDGNQELLNFLRSDLIEVATRLQKGGLGYVEETSEFEARVISLEKLKDFGECVIALQASVIKKFLQGFMAPKQKRRKLQSEDSAKTEEVDEEKKMVEEAKVASALEKWKTAIREAQTFSRMHVLLGMLDACIKWDMSAENARCKVCRKKGEDDKLILCDECNKAFHLFCLRPALYEVPDGEWQCPACQPATARRNSRGRNYTEESASEDSEDDESDEEEEEEEEEEEEEDYEVAGLRLRPRKTIRGKHSVIPPAARSGRRPGKKPHSTRRSQPKAPPVDDAEVDELVLQTKRSSRRQSLELQKCEEILHKIVKYRFSWPFREPVTRDEAEDYYDVITHPMDFQTVQNKCSCGSYRSVQEFLTDMKQVFTNAEVYNCRGSHVLSCMVKTEQCLVALLHKHLPGHPYVRRKRKKFPDRLAEDEGDSEPEAVGQSRGRRQKK

SEQ ID NO: 4 - protein, 6 aa, Artificial
  KENSSQ motif
KENSSQ

SEQ ID NO: 5 - DNA, 1173 bp, Artificial
  WSTF kinase domain
atggcgccgctcctgggccgcaagcccttcccgctggtgaagccgttgcccggagaggagccgctcttcaccatcccgcacactcaggaggccttccgcacccgggaagagtatgaagcccgcttggaaaggtacagtgagcgcatttggacgtgcaagagtactggaagcagtcagctaacacacaaggaagcctgggaggaagaacaggaagttgctgagcttttgaaggaggagtttcctgcctggtatgagaagcttgttctggaaatggttcaccataacacagcctccttagagaagttagtagatactgcttggttggagatcatgaccaaatatgctgtgggagaagagtgtgacttcgaggttgggaaggagaaaatgctcaaggtgaagattgtgaagattcatcctttggagaaagtggatgaagaggccactgagaagaaatctgatggtgcctgtgattctccatcaagtgacaaagagaactccagtcagattgctcaggaccatcagaagaaggagacagttgtgaaagaggatgaaggaaggagagagagtattaatgacagagcacgtagatcgccacgaaaacttcctacttcattaaaaaaaggagaaaggaaatgggctcctccaaaatttctgcctcacaaatatgatgtgaaactacaaaatgaagataagatcatcagtaacgtgccagcagacagcttgattcgtacagagcgcccaccaaataaggagatagttcgatactttatacggcataatgcattacgagctggtactggtgaaaatgcaccttgggtcgtagaagatgaattggtgaagaaatactctctgcccagcaagttcagtgactttttacttgatccatacaagtatatgactctcaacccttctactaagaggaagaatactggatccccagacaggaagccctcaaagaaatccaagacagacaactcttctcttagttcaccactaaatcctaagttatggtgtcacgtacacttgaagaagtcattgagtggctcgccactcaaagtgaagaactcaaagaattccaaatctcctgaagaacatctagaagaaatgatgaagatgatgtcgcccaataagctgcacactaactttcacattcctaaaaaaggcccacctgcc

SEQ ID NO: 6 - protein, 391 aa, Artificial
  WSTF kinase domain
MAPLLGRKPFPLVKPLPGEEPLFTIPHTQEAFRTREEYEARLERYSERIWTCKSTGSSQLTHKEAWEEEQEVAELLKEEFPAWYEKLVLEMVHHNTASLEKLVDTAWLEIMTKYAVGEECDFEVGKEKMLKVKIVKIHPLEKVDEEATEKKSDGACDSPSSDKENSSQIAQDHQKKETVVKEDEGRRESINDRARRSPRKLPTSLKKGERKWAPPKFLPHKYDVKLQNEDKIISNVPADSLIRTERPPNKEIVRYFIRHNALRAGTGENAPWVVEDELVKKYSLPSKFSDFLLDPYKYMTLNPSTKRKNTGSPDRKPSKKSKTDNSSLSSPLNPKLWCHVHLKKSLSGSPLKVKNSKNSKSPEEHLEEMMKMMSPNKLHTNFHIPKKGPPA

SEQ ID NO: 7 - protein, 367 aa, Artificial
  WSTF kinase domain consensus sequence
XXXXXXXKXXXXXXXXXXXXXXFXXXXXXXXXXXXXEEYEXRLERYXERIWTCKSTGSSQLTHXXXXXXEXEVXELLKEEFPXWXEKLVLEXVHHNTXSLEKLVDXAWXEIXTKXAVGEXCDFXVGXXKXLXXKIVKXHPLXXXXXXXXXXXXXXXXXXXXXKENSSQXXXXXXXXXXXXXXXXXXXXXXXXXXXXXXXXXXXXXKKXXXKWXPPKFLPHKYDVKLXNEDKIISXVPADXLXRTERPPNKEIXRYFIRHNALRAGXGEXXPWVVEDELVKKYXLPSKFSDFLLDPXKXXXXNPSTKRKXXGSPXXKPSKKXKXXXXSXXXXXXXXXWXXXXXXXXXXXXXXXXXXXXXXXXXXXXXX

SEQ ID NO: 8 - protein, 405 aa, Artificial
  WSTF kinase domain
GPLGSHMAPLLGRKPFPLVKPLPGEEPLFTIPHTQEAFRTREEYEARLERYSERIWTCKSTGSSQLTHKEAWEEEQEVAELLKEEFPAWYEKLVLEMVHHNTASLEKLVDTAWLEIMTKYAVGEECDFEVGKEKMLKVKIVKIHPLEKVDEEATEKKSDGACDSPSSDKENSSQIAQDHQKKETVVKEDEGRRESINDRARRSPRKLPTSLKKGERKWAPPKFLPHKYDVKLQNEDKIISNVPADSLIRTERPPNKEIVRYFIRHNALRAGTGENAPWVVEDELVKKYSLPSKFSDFLLDPYKYMTLNPSTKRKNTGSPDRKPSKKSKTDNSSLSSPLNPKLWCHVHLKKSLSGSPLKVKNSKNSKSPEEHLEEMMKMMSPNKLHTNFHIPKKGPPALEHHHHHH

SEQ ID NO: 9 - protein, 367 aa, Artificial
  Human WSTF kinase domain
MAPLLGRKPFPLVKPLPGEEPLFTIPHTQEAFRTREEYEARLERYSERIWTCKSTGSSQLTHKEAWEEEQEVAELLKEEFPAWYEKLVLEMVHHNTASLEKLVDTAWLEIMTKYAVGEECDFEVGKEKMLKVKIVKIHPLEKVDEEATEKKSDGACDSPSSDKENSSQIAQDHQKKETVVKEDEGRRESINDRARRSPRKLPTSLKKGERKWAPPKFLPHKYDVKLQNEDKIISNVPADSLIRTERPPNKEIVRYFIRHNALRAGTGENAPWVVEDELVKKYSLPSKFSDFLLDPYKYMTLNPSTKRKNTGSPDRKPSKKSKTDNSSLSSPLNPKLWCHVHLKKSLSGSPLKVKNSKNSKSPEEHLE

SEQ ID NO: 10 - protein, 367 aa, Artificial
  mouse WSTF kinase domain
MAPLLGRKPFPLVKPLPGEEPLFTIPHTQEAFRTREEYEARLERYSERIWTCKSTGSSQLTHKEAWEEEQEVAELLKEEFPNWYEKLVLEMVHHNTASLEKLVDSAWLEIMTKYAVGEECDFEVGKEKMLKVKIVKIHPLEKVDEEAVEKKSDGACDSPSSDKENSSQMAQDLQKKETVVKEDEGRRESINDRARRSPRKLPTSLKKGERKWAPPKFLPHKYDVKLQNEDKIISNVPADSLIRTERPPNKEILRYFIRHNALRAGTGENAPWVVEDELVKKYSLPSKFSDFLLDPYKYMTLNPSTKRRNTGSPDRKPSKKPKRDSSSLSSPLNPKLWCHVHLEKSLNGPPLKVKNSKNSKSPEEHLE

SEQ ID NO: 11 - protein, 372 aa, Artificial
  chicken WSTF kinase domain
MAPLLGRKPFPLAKPLPPGEPGERFIIPHTQEAFRTTCCCYREYEARLERYSERIWTCKSTGSSQLTHKEAWEEEQEVAELLKEEFPIWYEKPVLEIVHHNTVSLEKLVDAAWVEIMTKFAVGEECDFEVGKEKMLPVKVVKIHPLEKVDEEASEKKSEGACDSPSSDKENSSQAAQDNQKESVLKEEDSRRESMNDRARRSPRKLPTSLKKEERKWVPPKFLPHKYDVKLKNEDKIISNVPADSLVRTERPPNKEILRYFIRHNALRAGIGENAPWVVEDELVKKYSLPSKFSDFLLDPHKYMTLNPSTKRKSSGSPDRKPSKKSKTDGSSLSQPLSPTLWCHVHLEKSIIGSPLKMKNSKISKCPKEELE

SEQ ID NO: 12 - protein, 381 aa, Artificial
  xenopus WSTF kinase domain
MAPLLGRRPFPLVKPLSEAATGEGEEEVYMIEHSKEAFRSREEYESRLERYAERIWTCKSTGSSQLTHKEAWDEEQEVAELLKEEFPVWYEKQVLEMVHHNTISLDKLVDQSWMEIMTKYADGEECDFEVGPEKYLRAKIVKVHPLEKEEQASEKKSEGSCDSPSSDKENSNKVAQDIQLKEESNRLESLSSLRESDRARRSPRKLPTSLKKEEKKWVPPKFLPHKYDVKLLNEDKVISFVPVDSLYRSERPPNKEILRYFIRHNALRIGTGENPPWVVEDELVKKYTLPSKFSDFLLDPHKYMTLNPSSATKRKSLGSPDQKPAKKSKKSPLSPSSWSLANLKKTAVNSSSSEEEMQLMIGANLNKKGSIGKKSDKKKPK

SEQ ID NO: 13 - protein, 383 aa, Artificial
  zebrafish WSTF kinase domain
MAPLLGRKPYPLVKPLSEPPGPGEEVYTIEHTKEAFRNKEEYEARLRRYGERIWTCKSTGSSQLTHKEAWEEEQEVTELLQEEYPVWFEKPVLEIVHHNTVPLDKLVDQVWVEILTKYAVGEKCDLMVGNDKTLSVEVVKIHPLENPPEENAEKKMEGACDSPSSDKENASQENLKKEPQSKEEESRRESLSDRARRSPRKLPTTMKEEKKKWVMPKFLPHKYDVKLVNEDKVISDVPADNLFRTERPPNKEIMRYFIRHYALRLGSGESAPWVVEDELVKKFSLPSKFSDFLLDPHKFLAENPSTKRKSLSSPEGKPRKRLKNVETGTGGEGAKGDKKKNKDSQNIPLSPTIWSHMQVKKVNGSPLKMKNSGTSKKSDEENV

SEQ ID NO: 14 - protein, 431 aa, Artificial
  Ciona savignyi WSTF kinase domain
MPMLGRKTFDGGSPSLGGIESKFVIPHTKEAFGDEEEYTERMNLYDKKIWTCQCTGHSNHTHLGATLSEASCREKLAQTYDEIWEEPVLKLVHHSFSPLESIVVQANNTLLTTLVHGETVKFTAKPGVTFLAKIQEKHVDVDVPETSTDPDTPDVIDKKEDVAENGDKTDETNHDGKEDSVKSEVGENSSDAKKILNSVSPSSDKENKRQSLITDIFKTDIRRSARSPSTIVRSGVSLKGKRPKIIPAKYNILLVEENKIVSKVPSEHLTRTMKPPNRDQIRLFIRSNSIRPLSSKKKVPWMVNEDLVFRYKLPSRVPEEEIVDILQLCSVTTQQYLHGSDTSISNGHCNGDVIDVDDCDETSKKRKGEKLQSQSKKAKRDSLLLKETKSSLDAFLKKTTKLFSEKIGEIDGKSARQQGSPRKSTSPKKKK

SEQ ID NO: 15 - protein, 352 aa, Artificial
  Aplysia californica WSTF kinase domain
MPLLGKKVFALGAQSPNNEQEDAPYLIPHTKERFQSKTEYEKRVNLYAQLIWTCRATGHTCLTHEEACKSEQVVTDQLKSQFPKCFEKEVLSQVHHSILSLDSLVDKTWWTLHQQFVVGEKVSLKVKVADKLIPAKIVGVDSSSNEAKSGATCNSPSSDKENSSEEKDGNKSPKKWVPPKFLPYTYSIELENEAKIIHSVPSADLMRDEKAPSKDLLRLYIRVSAIRSGTLSTSPWVANADLVKQYKLISKFADFFLSPMKMADAFKKAEETGKKRKLSNGMVGSVAKKLKLEKSSEKPKKTKDKSKTPQGSNKADKNPSSKKKKNLNKDSNKSSAKKTSPSKKAATTGKKA

SEQ ID NO: 16 - protein, 351 aa, Artificial
  Lottia gigantean WSTF kinase domain
MPLLGKKIYSPSKPPKDILPNEKVFTIPFTGERFRSKSEYDKRCSLYREKIWTCQCTGHVNLSYEEAHKSEKAGHKTIKDQFDECFERPTLEVVHHATVSLDNLVDQAWTKIQQVLLVGENVALKVKAAGRTIKGTIVSVDNCGMDVNPTSNCSSPSSDKENNNSKDQPKKWQAPKLLPYKYSMRLEGEEKVINAIPASDLARCGRPPSKDLIRLFIRAHTVRAGQSATSPWVVDDKMVHKYKLPKKFTDFLLSPAKMTRITPQMVEEISKKRKMEVEVVESSSDEDEIVLSEVIRKSIGKSDASANNDSDSDVPLVKLKSKNNSNGSDSEDEPLSKLGSGKKSPKKKSPT

SEQ ID NO: 17 - protein, 272 aa, Artificial
  Paracentrotus lividus WSTF kinase domain
MPLLGRKLYIPKVLSKTVTLAEPHFLIPFTNEVFSSKIDYDNQLEVYAKALWTCQCTGQTGLTFEEAQRSEVNARQRLQDFPAHFEKPILQHVHHNASTMEILLQETTDSLISSFVQGEKVELKVKVKGKLFRGKIVKKVEADSTSEVPGVSGSPSSDKENHGNSSDEGLSSKTKKKNQSSSYRYNIQLENEEKIISSVPFSDLIRCNPLPSRDILEIFIRCSALQAGNKPASPWIVHDDLVKKHALSGRLIPSLLSPTRISATGLKLAVKR

SEQ ID NO: 18 - protein, 4 aa, Artificial
  D. melanogaster SQAY motif
SQAY

SEQ ID NO: 19 - protein, 4 aa, Artificial
  X. laevis SQEY/F motif
SQEX

SEQ ID NO: 20 - protein, 4 aa, Artificial
  S. cerevisia SQEL
SQEL

SEQ ID NO: 21 - DNA, 97 bp, Artificial
  WSTF kinase domain shRNA
tgctgttgacagtgagcgcggagatacttcgatactttattagtgaagccacagatgtaataaagtatcgaagtatctccttgcctactgcctcgga

SEQ ID NO: 22 - DNA, 35 bp, Artificial
  primer
caagaaggccacccaggcctcccaggagctctaag

SEQ ID NO: 23 - DNA, 35 bp, Artificial
  primer
cttagagctcctgggaggcctgggtggccttcttg

SEQ ID NO: 24 - DNA, 35 bp, Artificial
  primer
caagaaggccacccaggcctcccaggagttctaag

SEQ ID NO: 25 - DNA, 35 bp, Artificial
  primer
cttagaactcctgggaggcctgggtggccttcttg

SEQ ID NO: 26 - DNA, 23 bp, Artificial
  primer
cacctcgggccgcggcaagactg

SEQ ID NO: 27 - DNA, 17 bp, Artificial
  primer
cttagtactcctgggag

SEQ ID NO: 28 - DNA, 17 bp, Artificial
  primer
cttagagctcctgggag

SEQ ID NO: 29 - DNA, 17 bp, Artificial
  primer
cttagaactcctgggag

SEQ ID NO: 30 - protein, 15 aa, Artificial
  unmodified peptide
CPSGGKKATQASQEY

SEQ ID NO: 31 - protein, 15 aa, Artificial
  S139(ph)
CPSGGKKATQASQEY

SEQ ID NO: 32 - protein, 15 aa, Artificial
  Y142(ph)
CPSGGKKATQASQEY

SEQ ID NO: 33 - protein, 15 aa, Artificial
  S139(ph) and Y142(ph)
CPSGGKKATQASQEY

SEQ ID NO: 34 - protein, 15 aa, Artificial
  F142
CPSGGKKATQASQEF

SEQ ID NO: 35 - protein, 15 aa, Artificial
  L142
CPSGGKKATQASQEL

SEQ ID NO: 36 - protein, 15 aa, Artificial
  S139(ph), L142
CPSGGKKATQASQEL

SEQ ID NO: 37 - protein, 15 aa, Artificial
  S139(ph), F142
CPSGGKKATQASQEF

SEQ ID NO: 38 - protein, 8 aa, Artificial
  WSTF peptide
ILTALCHR

SEQ ID NO: 39 - protein, 8 aa, Artificial
  WSTF peptide
ARLPEELR

SEQ ID NO: 40 - protein, 9 aa, Artificial
  WSTF peptide
SDLIEVATR

SEQ ID NO: 41 - protein, 9 aa, Artificial
  WSTF peptide
HLPGHPYVR

SEQ ID NO: 42 - protein, 10 aa, Artificial
  WSTF peptide
GSHVLSCMEK

SEQ ID NO: 43 - protein, 10 aa, Artificial
  WSTF peptide
GWVHNSIDYR

SEQ ID NO: 44 - protein, 12 aa, Artificial
  WSTF peptide
IISNVPADSLIR

SEQ ID NO: 45 - protein, 13 aa, Artificial
  WSTF peptide
RDSSSLSSPLNPK

SEQ ID NO: 46 - protein, 13 aa, Artificial
  WSTF peptide
STGNSKSPSKCVK

SEQ ID NO: 47 - protein, 11 aa, Artificial
  WSTF peptide
KRWASMSEEQR

SEQ ID NO: 48 - protein, 11 aa, Artificial
  WSTF peptide
FSWPFREPVTR

SEQ ID NO: 49 - protein, 14 aa, Artificial
  WSTF peptide
HLPPAALHLIAYYK

SEQ ID NO: 50 - protein, 16 aa, Artificial
  WSTF peptide
GPAAKKPGKHSDKPLK

SEQ ID NO: 51 - protein, 16 aa, Artificial
  WSTF peptide
WDMSAENARCKVCRKK

SEQ ID NO: 52 - protein, 17 aa, Artificial
  WSTF peptide
SRPKDDPEVDDLVLQTK

SEQ ID NO: 53 - protein, 18 aa, Artificial
  WSTF peptide
LQNEDKIISNVPADSLIR

SEQ ID NO: 54 - protein, 21 aa, Artificial
  WSTF peptide
NASVNAHHGPALEAVETTVPK

SEQ ID NO: 55 - protein, 22 aa, Artificial
  WSTF peptide
SVQEFLTDMKQVFANAELYNCR

SEQ ID NO: 56 - protein, 24 aa, Artificial
  WSTF peptide
LQILTALCHRILMTYSVQDHMETR

SEQ ID NO: 57 - protein, 9 aa, Artificial
  SMARC peptide
YLVIDEAHR

SEQ ID NO: 58 - protein, 9 aa, Artificial
  SMARC peptide
ANRFEYLLK

SEQ ID NO: 59 - protein, 9 aa, Artificial
  SMARC peptide
YKAPFHQLR

SEQ ID NO: 60 - protein, 10 aa, Artificial
  SMARC peptide
FITDNTVEER

SEQ ID NO: 61 - protein, 11 aa, Artificial
  SMARC peptide
KANYAVDAYFR

SEQ ID NO: 62 - protein, 11 aa, Artificial
  SMARC peptide
IGKDEMLQMIR

SEQ ID NO: 63 - protein, 12 aa, Artificial
  SMARC peptide
LRLDSIVIQQGR

SEQ ID NO: 64 - protein, 13 aa, Artificial
  SMARC peptide
DIDILNSAGKMDK

SEQ ID NO: 65 - protein, 12 aa, Artificial
  SMARC peptide
IAFTEWIEPPKR

SEQ ID NO: 66 - protein, 13 aa, Artificial
  SMARC peptide
QPNVQDFQFFPPR

(19) United States
(12) Patent Application Publication
(10) Pub. No.: US 2010/0080799 A1
(43) Pub. Date: Apr. 1, 2010

(54) WSTF REGULATES THE DNA DAMAGE RESPONSE OF H2A.X VIA NOVEL TYROSINE KINASE ACTIVITY

(75) Inventors: C. David Allis, Princeton, NJ (US); Andrew Xiao, New York, NY (US)

Correspondence Address: NIXON PEABODY LLP - PATENT GROUP, 1100 CLINTON SQUARE, ROCHESTER, NY, 14604, US

(73) Assignee: THE ROCKEFELLER UNIVERSITY, New York, NY (US)

(21) Appl. No.: 12/561,573

(22) Filed: Sep. 17, 2009

Related U.S. Application Data
(60) Provisional application No. 61/097,672, filed on Sep. 17, 2008.

(51) Int. Cl. A61K 39/395; C07K 16/00; C07K 14/00; C07H 21/00; C12N 15/74; C12N 1/21; C12Q 1/68; G01N 33/53; C12N 5/071; A61K 31/7105; A61K 38/46; A61K 31/711; A61P 35/00

(52) U.S. Cl. 424/130.1; 530/387.1; 530/388.1; 530/389.1; 530/387.3; 530/350; 536/23.1; 435/320.1; 435/253.3; 435/6; 435/7.2; 435/325; 514/44 A; 424/94.6; 514/44 R

(57) ABSTRACT

The present invention relates to methods of detecting and regulating cellular DNA damage as well as methods of screening compounds suitable for modulating cellular DNA damage. The methods of the present invention utilize antibodies that selectively bind to a phosphorylated tyrosine residue in a SQEY tyrosine phosphorylation motif sequence or a phosphorylated serine residue in a KENSSQ phosphorylation motif sequence, where the phosphorylated serine residue is closest to the glutamine residue. Also encompassed by the present invention are methods of treating a subject having cancer. These methods involve the administration of an agent that modulates the phosphorylation of the tyrosine residue in a SQEY motif. Suitable agents for modulating the phosphorylation of the tyrosine residue of the SQEY motif are also disclosed.

This application claims the benefit of U.S. Provisional Patent Application Ser. No. 61/097,672, filed Sep. 17, 2008, which is hereby incorporated in its entirety.

The subject matter of this application was made with support from the United States Government under the National Institutes of Health, Grant No. R01-GM40922. The government has certain rights in this invention.

### FIELD OF THE INVENTION

The present invention describes methods of identifying and regulating cellular DNA damage repair and methods of treating a subject having cancer. These methods involve modulating tyrosine phosphorylation at a newly defined tyrosine phosphorylation motif. The invention further describes a novel tyrosine kinase involved in regulating DNA damage repair.

### BACKGROUND OF THE INVENTION

One hallmark of the mammalian DNA double-strand break (DSB) response is the formation of Ionizing Radiation Induced Foci (IRIF), which are composed of compacted chromatin and numerous DNA repair and checkpoint proteins (Khanna et al., “DNA Double-Strand Breaks: Signaling, Repair and the Cancer Connection,” Nat Genet. 27:247-254 (2001) and Zhou et al., The DNA Damage Response Putting Checkpoints in Perspective,” Nature 408:433-439 (2000)). Despite considerable progress in understanding signaling pathways leading to checkpoint control and DNA repair, the nature of the specialized chromatin structures at IRIF is not well understood. One of the earliest events occurring at IRIF is the phosphorylation of H2A.X, a specialized histone H2A variant, at Ser139 (referred to as γ-H2A.X) by the ATM and ATR kinases (Redon et al., “Histone H2A Variants H2AX and H2AZ,” Curr Opin Genet Dev 12:162-169 (2002)). H2A.X-deficient mouse embryonic fibroblasts (MEFs), B and T cells display pronounced levels of genomic instability, including broken and dicentric chromosomes and random translocations (Celeste et al., “Genomic Instability in Mice Lacking Histone H2AX,” Science 296:922-927 (2002)). Class switch recombination and spermatogenesis are also defective in H2A.X deficient mice, further implying its involvement in DNA damage repair under physiological conditions (Celeste et al., “Genomic Instability in Mice Lacking Histone H2AX,” Science 296:922-927 (2002); Celeste et al., “H2AX Haploinsufficiency Modifies Genomic Stability and Tumor Susceptibility,” Cell 114:371-383 (2003); and Reina-San-Martin et al., “H2AX is Required for Recombination Between Immunoglobulin Switch Regions but Not for Intra-Switch Region Recombination or Somatic Hypermutation,” J Exp Med 197:1767-1778 (2003)). Moreover, H2A.X-deficiency accelerates B and T cell lymphoma development in p53-deficient mice (Celeste et al., “H2AX Haploinsufficiency Modifies Genomic Stability and Tumor Susceptibility,” Cell 114:371-383 (2003);
Reina-San-Martin et al., “H2AX is Required for Recombination Between Immunoglobulin Switch Regions but Not for Intra-Switch Region Recombination or Somatic Hypermutation,” J Exp Med 197:1767-1778 (2003); and Bassing et al., “Histone H2AX: A Dosage-dependent Suppressor of Oncogenic Translocations and Tumors,” Cell 114:359-370 (2003)). Consistent with these functions in mammalian cells, phosphorylation of the equivalent site on yeast H2A (Ser129) is found at DSB sites and spreads to ±50 Kb of the flanking regions (Unal et al., “DNA Damage Response Pathway Uses Histone Modification to Assemble a Double-Strand Break-Specific Cohesin Domain,” Mol Cell 16:991-1002 (2004)). In mammals this phosphorylation event directly recruits Mdc1 and sets in motion the recruitment of additional factors such as 53BP1, RNF8, and the Brca1 A complex (Harper et al., “The DNA Damage Response: Ten Years After,” Mol Cell 28:739-745 (2007)). Furthermore, several recent studies have also indicated that ATP-dependent chromatin remodeling complexes are engaged in DNA repair pathways at these sites. The yeast NuA4 and INO80 complexes, for example, are recruited to the damaged chromatin via γ-H2A.X (Morrison et al., “INO80 and Gamma-H2AX Interaction Links ATP-dependent Chromatin Remodeling to DNA Damage Repair,” Cell 119:767-775 (2004); Downs et al., “Binding of Chromatin-Modifying Activities to Phosphorylated Histone H2A at DNA Damage Sites,” Mol Cell 16:979-990 (2004); and van Attikum et al., “Recruitment of the INO80 Complex by H2A Phosphorylation Links ATP-Dependent Chromatin Remodeling With DNA Double-Strand Break Repair,” Cell 119:777-788 (2004)).

Mammalian H2A.X bears significant differences with lower eukaryotes. For example, H2A.X is a minor H2A variant in mammalian cells (1-10%) while the major yeast form of H2A is most similar to H2A.X, because it contains the signature C-terminal sequence of mammalian H2A.X (Redon et al., “Histone H2A Variants H2AX and H2AZ,” Curr Opin Genet Dev 12:162-169 (2002)). A Drosophila H2A variant, known as H2A.V, is a “hybrid” protein containing signature sequences of mammalian H2A.X and H2A.Z (Redon et al., “Histone H2A Variants H2AX and H2AZ,” Curr Opin Genet Dev 12:162-169 (2002)). The Drosophila Tip60 complex exchanges phosphorylated H2A.V with unmodified H2A.V in vitro and regulates H2A.V phosphorylation levels in vivo (Kusch et al., “Acetylation by Tip60 is Required for Selective Histone Variant Exchange at DNA Lesions,” Science 306:2084-2087 (2004)). In mammalian cells, the loss of SWI/SNF chromatin remodeling complex expression leads to defects in the H2A.X DNA damage response. However, it is unclear whether these defects are due to the lack of direct regulation by the SWI/SNF complex or other indirect pathways, since the SWI/SNF complex is involved in a variety of important biological processes (Park et al., “Mammalian SWI/SNF Complexes Facilitate DNA Double-Strand Break Repair by Promoting Gamma-H2AX Induction,” EMBO J 25:3986-3997 (2006)).

Given the unique features of H2A.X in mammalian cells, factors that are directly involved in regulating H2A.X function need to be identified.

### SUMMARY OF THE INVENTION

A first aspect of the present invention relates to an antibody or antigen-binding fragment thereof that selectively binds to a phosphorylated tyrosine residue in an SQEY tyrosine phosphorylation motif sequence (SEQ ID NO: 2).

A second aspect of the present invention relates to an antibody or antigen-binding fragment thereof that selectively binds to the phosphorylated serine residue closest to the glutamine residue in an KENSSQ phosphorylation motif sequence (SEQ ID NO:4).

Other aspects of the present invention relate to an isolated polypeptide molecule having an amino acid sequence of SEQ ID NO: 6 or SEQ ID NO: 7, and an isolated nucleic acid molecule encoding the polypeptide.

Another aspect of the present invention relates to a method of identifying cellular DNA damage in a sample. This method involves providing a cell sample and detecting the presence or absence of a phosphorylated tyrosine residue in an SQEY tyrosine phosphorylation motif (SEQ ID NO:2). Cellular DNA damage in the cell sample is identified based on detecting the presence or absence of tyrosine phosphorylation.

Another aspect of the present invention relates to a method of identifying cellular DNA damage in a sample. This method involves providing a cell sample and detecting the presence or absence of a phosphorylated serine residue at the serine closest to the glutamine residue in an KENSSQ phosphorylation motif sequence (SEQ ID NO:4). Cellular DNA damage in the cell sample is identified based on detecting the presence or absence of serine phosphorylation.

Another aspect of the present invention relates to a method of screening candidate compounds useful for modulating WSTF kinase activity. This method involves providing a candidate compound and a sample, where the sample contains a WSTF protein or polypeptide having kinase activity, a protein substrate having a SQEY motif sequence, manganese, and ATP. Contacting the candidate compound with the sample and detecting the presence or absence of a phosphorylated tyrosine residue in a SQEY tyrosine phosphorylation motif (SEQ ID NO:2) identifies compounds useful for modulating WSTF kinase activity.

The present invention is also directed to a method of screening candidate compounds useful for modulating cellular DNA damage repair. This method involves providing a candidate compound and a cell sample and contacting the candidate compound with the cell sample. The presence or absence of a phosphorylated tyrosine residue in a SQEY tyrosine phosphorylation motif (SEQ ID NO:2) in the cell sample is detected, and candidate compounds useful for modulating cellular DNA damage repair are identified based on the presence or absence of tyrosine phosphorylation.

Another aspect of the present invention relates to a method of screening candidate compounds useful for treating cancer. This method involves providing a candidate compound and a cell sample and contacting the candidate compound with the cell sample. The presence or absence of a phosphorylated tyrosine residue in a SQEY tyrosine phosphorylation motif (SEQ ID NO:2) in the cell sample is detected and candidate compounds useful for treating cancer are identified based on the presence or absence of tyrosine phosphorylation.

The present invention is also directed to a method of modulating cellular DNA damage repair. This method involves administering to a cell an agent which modulates tyrosine phosphorylation of a SQEY tyrosine phosphorylation motif (SEQ ID NO:2) under conditions effective to modulate cellular DNA damage repair.

Another aspect of the present invention is directed to a method of treating a subject having cancer. This method involves administering to the subject having cancer, an agent that modulates tyrosine phosphorylation of a SQEY tyrosine phosphorylation motif (SEQ ID NO:2) under conditions effective to treat the subject having cancer.

The studies described herein define a novel DNA damage response pathway regulating H2A.X function in mammals that is mediated through the WSTF-SNF2H chromatin remodeling complex and involves the phosphorylation of H2A.X at tyrosine residue 142 (“Tyr142” or “Y142”). Unexpectedly, it was determined that the amino-terminal domain of WSTF, including its WAC domain, exhibits tyrosine kinase activity towards Tyr142 of H2A.X, a novel phosphorylation mark that plays a role in γ-H2A.X maintenance and related downstream chromatin remodeling events. Both the novel tyrosine kinase activity of WSTF and the phosphorylation of H2A.X at Tyr142 are required for eliciting an appropriate downstream DNA damage response in mammalian cells. These findings support an emerging view that the regulatory pathways of chromatin structure brought about by histone covalent modification, chromatin remodeling by ATP-dependent complexes, and the introduction of histone variants are closely interconnected, affecting many DNA-templated processes including the repair of double-strand breaks.

### BRIEF DESCRIPTION OF THE DRAWINGS

FIGS. 1A-1B show Tyr142, a novel phosphorylation mark of H2A.X, is regulated by DNA damage signals. FIG. 1A is a comparison of extreme C-terminal sequences of H2A.X of various species, demonstrating that Tyr142 is conserved in “multicellular” (mammals and fruit flies) but not in “single-cellular” eukaryotes. As shown in FIG. 1A, the SQEY motif of SEQ ID NO:2 is conserved between H. sapiens and M. musculus; D. melanogaster has an SQAY motif (SEQ ID NO: 18); X. laevis has an SQEY/F motif (SEQ ID NO: 19); and S. cerevisiae has a SQEL motif (SEQ ID NO: 20). In FIG. 1B, primary MEF cells were treated with 10 Gy of ionizing radiation (IR) and recovered for the period of time indicated. Acid-extracted histones were separated by SDS-PAGE and subjected to immunoblotting. H2A.X Tyr142 phosphorylation levels in MEFs gradually declined, reaching a minimum at 8 hr. As expected, γ-H2A.X signal was initiated upon damage and maintained up to 16 hours post IR.

FIGS. 2A-2D demonstrate the specific association between the WSTF-SNF2H chromatin remodeling complex and H2A.X nucleosomes in vivo. FIG. 2A shows the purification scheme of H2A.X-containing mononucleosomes. MEFs reconstituted with Flag-H2A.X (WT or Y142F mutant) were treated with or without ionizing radiation. After hypotonic buffer extraction, nuclear pellets (P1) were further extracted and fractionated into soluble nuclear extracts (S2) and insoluble nuclear pellets (P2) containing primarily chromatin and associated proteins. After Mnase digestion, mononucleosomes were immunoprecipitated with α-Flag to enrich for H2A.X-containing nucleosomes and associated proteins. This approach efficiently and specifically isolated H2A.X nucleosomes (See FIG. 6). The immunoprecipitated complexes were separated by SDS-PAGE and silver stained (FIG. 2B upper panels). Two polypeptides migrating at 145 and 171 KDa were consistently associated with undamaged WT (FIG. 2B, upper left panel), but not the Y142F mutant (FIG. 2B, upper right panel) H2A.X mononucleosomes. Mass spectrometry (MS) analyses demonstrated that these polypeptides, WSTF (171 KDa) and SNF2H (145 KDa), constitute the mammalian WICH complex (See FIG. 7). The third band at 60 KDa was identified as β-actin in the MS analyses; the significance of β-actin is not known. An aliquot of the complex immunoprecipitated from the same sample was separated by SDS-PAGE and silver stained for lower molecular weight bands (<20 KDa) (FIG. 2B, lower panel). Staining revealed similar levels of H2A.X and other core histones identified by MS analysis. In FIG. 2C the association between the WICH complex and undamaged WT H2A.X mononucleosomes was confirmed by IP-western experiments. Note that the 5139 phosphorylation level was greatly reduced in the Y142F mutant cells (FIG. 2C; also see FIG. 4). WSTF knock-down cell lines (WSTF RNAi) were generated by stably expressing short hairpin RNAi constructs specifically targeting the WSTF gene in NIH3T3 cells. FIG. 4D shows SDS-PAGE and immunoblot of nuclear extracts from the WSTF RNAi and control 3T3 cells. In WSTF RNAi cells, the expression level of WSTF was significantly diminished and H2A.X Y142 phosphorylation level was significantly reduced.

FIGS. 3A-3G illustrate the characterization of the novel kinase domain of WSTF that phosphorylates Tyr142 of H2A.X. FIG. 3A is a schematic representation of the domain architecture of the human WSTF protein and a series of recombinant proteins representing portions of WSTF (i.e., the 1-345N construct represents N-terminal amino acids 1 to 345 of WSTF). These constructs were extensively purified through affinity (GST pull down), ion exchange (SP sepharose) and sizing (gel filtration) chromatography. No other proteins at significant level (>2.5%) were co-purified with them, as confirmed by MS analysis. In FIGS. 3B-F, recombinant WSTF proteins were generated in insect cells and the N- and C-terminal motifs (FIG. 3G) were generated in E. coli. As shown in FIG. 3B, Y142 was phosphorylated by recombinant full length WSTF proteins. WT and Y142F mutant H2A.X nucleosomes were reconstituted with recombinant free histones and DNA molecules in vitro. Following in vitro kinase assays, reaction mixtures were separated by SDS-PAGE and exposed for radioautography. In FIG. 3C, the specific phosphorylation of H2A.X at Tyr142 was also detected by immunoblotting with an α-H2A.X Y142ph antibody. In FIGS. 3D-G, free H2A.X proteins were used as substrates in in vitro kinase assays. The recombinant N-terminal (N-WSTF) and C-terminal portion (C-WSTF) of WSTF were generated from insect cells and tested for kinase activity. Only N-WSTF had kinase activity as shown in FIG. 3D. A series of truncated WSTF proteins were generated from insect cells (see FIG. 3A) and the activity of the 1-340 amino acid construct was much reduced (<50 fold) in comparison to the other constructs (FIG. 3E). The C338A point mutant was derived from the WT 1-359 construct and its kinase activity was much reduced compared to WT (FIG. 3F). The N-motif and C-motif of WSTF kinase domain were generated either individually or in combination from E. coli. The co-expressed N-motif and C-motif had significant kinase activity towards H2A.X, while the N-motif alone had a minimal kinase activity (FIG. 3G). The C-motif construct, when expressed alone, was unstable and partially degraded from its C-terminus, however, it was protected when co-expressed with the N-motif.

FIGS. 4A-4J demonstrate how critical WSTF is for the maintenance of γ-H2A.X phosphorylation after DNA damage. In WSTF RNAi cells (as described in FIG. 2), γ-H2A.X level maintenance was defective after DNA damage treatment as illustrated in FIG. 4A. Western blotting experiments were performed on the histone samples isolated from control and WSTF RNAi cells after 10 Gy of IR treatment. The time points labeled indicate the recovery time following the IR treatment. The maintenance of γ-H2A.X foci is also defective in WSTF RNAi cells as shown in FIG. 4B. Immunofluorescent staining experiments were performed on control and WSTF RNAi cells fixed at different time points (as labeled) following 10 Gy of IR treatment. As shown in FIGS. 4C and 4D, phos-ATM and Mdc1 recruitment is also defective in WSTF deficient cells. Immunofluorescent staining experiments were performed using anti-ATM S1981 phos (FIG. 4C) and anti-Mdc1 (FIG. 4D) antibodies on control or WSTF RNAi cells 8-hours post 10 Gy of IR treatment. FIGS. 4E and 4F demonstrates that WSTF kinase activity is also critical for γ-H2A.X and phos-ATM foci maintenance. FIGS. 4E and 4F are immunofluorescent images of WSTF RNAi cells complemented with wildtype or mutant (C338A) kinase domain constructs of WSTF (amino acids 1-359, myc epitope-tagged at N-terminus). Eight hours following treatment with 10 Gy of IR, cells were fixed and co-stained with anti-myc (red) and γ-H2A.X (green, FIG. 4E) or anti-ATM S1981 phos (green, FIG. 4F) antibodies. As shown in FIGS. 4G and 4H, the level of γ-H2A.X protein is reduced in Tyr142 mutants. H2A.X null MEFs were reconstituted with WT or Y142H2A.X mutants (Y to L or Y to F mutants). Histone samples were separated by SDS-PAGE and subjected to Western blotting (“V” is vector only) (FIG. 4G). Immunostaining experiments with these same cells demonstrated that γ-H2A.X foci (FIG. 4H, upper left panel) were observed in WT cells upon DNA damage while they were severely diminished in Tyr142 mutants (upper middle and right panels). Cells were fixed after DNA damage treatment (10 Gy 2 hr) and stained with γ-H2A.X antibodies. In peptide pull-down experiments (FIG. 4I), Ser139(ph) peptides specifically interacted with endogenous MDC1, but not BRCA1 from 293T cell nuclear extracts. Either pre-phosphorylation of Y142 (i.e., a doubly phosphorylated (S139(ph) & Y142(ph)) peptide) or replacement of the C-terminal tyrosine to leucine (L) or phenylalanine (F) diminished MDC1 binding significantly in this assay. A recombinant MDC1 BRCT domain specifically bound to Ser139(ph) peptides as demonstrated in FIG. 4J. Similarly, pre-phosphorylation or replacement of Y142 diminished binding significantly.

FIGS. 5A-5F show the modulation of Y142 phosphorylation level during DNA damage response and the characterization of the α-H2A.X Y142ph antibody. In FIG. 5A, α-H2A.X Y142ph antibodies were reacted with Xenopus H2A.X in Xenopus egg extracts. After incubating with double-strand break (DSB) plasmid DNA (for the time span as indicated), the Y142 phosphorylation significantly decreased while γ-H2A.X increased, reminiscent of mammalian cells. Yeast H2A L132Y mutant strongly reacted with antibodies to mammalian H2A.X Y142 phosphorylation (FIG. 5B). As expected, WT yeast cells did not have affinity for the anti-H2A.X Y142ph antibody. In contrast, the anti-H2A.X Y142ph antibody reacted with nuclear extracts from untreated H2A L132Y cultures, but did not react with extracts of cells treated with the DNA-damaging agent, MMS. γ-H2A.X (S139ph) antibody reacted strongly with WT and H2A L132Y cells following DNA damage. The equal loading of histone proteins was judged by α-H3 antibodies. In ELISA, α-H2A.X-Y142ph antibody reacted with H2A.X C-terminal peptides (129-142) that contained phosphorylated Tyr142 regardless of the phosphorylation status of Ser139 (FIG. 5C, compare (▴) and (x) curves), but not with similar peptides where Tyr142 was not phosphorylated (compare (♦) and (▪) curves). In western blot analyses, α-H2A.X Y142ph antibody recognized a single band at the expected molecular weight of H2A.X (˜17 KDa in U2OS cells (FIG. 5D, left lane)); this band was sensitive to protein tyrosine phosphatase b (FIG. 5D, right lane). The signals could be competed away only by H2A.X Y142ph peptides, but not unmodified peptides (FIG. 5E). In western blot analyses, this antibody only reacted with reconstituted WT H2A.X cells, but not cells expressing a Y142F point mutant (FIG. 5F).

FIG. 6 shows H2A.X-containing nucleosomes enriched in immunocomplexes precipitated with α-Flag. Flag-H2A.X-containing mononucleosomes isolated from reconstituted MEFs (see scheme in FIG. 2A) were separated by SDS-PAGE before being subjected to immunoblot analyses or Coomassie blue staining. In western blot analyses (upper panel), α-Flag antibodies recognized a single band at 20 KDa (there was no endogenous H2A.X in these cells and the MW of Flag-tagged H2A.X was larger than H3). Coomassie staining of the immunoprecipitated complexes (lower panel) revealed that Flag-H2A.X (MW 20 KDa) was associated with other core histones at similar stoichiometrical level whereas it was undetectable in input (5%) samples.

FIG. 7 is an identification of WSTF-SNF2H complex subunits by mass spectrometry. Protein bands (at 171 and 140 KDa, FIG. 2B) were isolated from SDS-PAGE gel and subjected to trypsin digestion. Peptide mass fingerprinting (PMF) demonstrated that in the protein band at 171 KDa, 19 peptides (SEQ ID NOs: 38-56; selected out of 29 for PMF) were from the mouse WSTF protein, which cover 248 out of 1479 amino acids (16.8%). In the protein band at 140 KDa, 10 peptides (SEQ ID NOs: 57-66; out of 35 selected for PMF) were from the mouse SNF2H protein, which cover 109 out of 1052 amino acids (10.4%). The protein band at 60 KDa was identified as β-actin.

FIG. 8 demonstrates that alteration of C-terminal Tyr142 in H2A.X does not change the affinity of γ-H2A.X antibodies. In ELISA, γ-H2A.X antibodies reacted with H2A.X C-terminal peptides (amino acid residues 129-142) that contain Phos-Ser139 regardless of the phosphorylation status of Tyr142 or Y142L replacement.

FIGS. 9A-9B show that phosphorylation of H2A.X at Ser139 by the ATM/R kinases upon DNA damage does not require Tyr142 and was not affected by phosphorylation at this site. H2A.X C-terminal peptides (listed in FIG. 9B) were incubated with Xenopus egg extracts in the presence of DSB DNA (+) or control DNA(−) and γ-³²P-ATP (reflective of ATM kinase; see Khanna et al., “DNA Double-Strand Breaks Signaling, Repair and the Cancer Connection,” Nat Genet 27:247-254 (2001), which hereby incorporated by reference in its entirely). Incorporation of radioactive ATP was measured by scintillation counting in standard P81 filter paper assays (Khanna et al., “DNA Double-Strand Breaks: Signaling, Repair and the Cancer Connection,” Nat Genet 27:247-254 (2001), which hereby incorporated by reference in its entirely). FIG. 9A is a bar graph showing ATM kinase activity (Ser139 phosphorylation). Ser139 of H2A.X was specifically phosphorylated in the presence of DSB DNA (compare unmodified peptide (UN) in the presence of DSB DNA (+) as compared to control DNA (−) or with pre-phosphorylated Ser139(ph) peptide (DSB+). The status of Tyr142, either in its pre-phosphorylated state (Tyr142(ph)) or a Tyr-to-Leu replacement (Leu142) failed to significantly affect Ser139 phosphorylation in the presence of DSB.

FIGS. 10A-10B demonstrate that the in vitro kinase activity of WSTF is dependent on Mn²⁺ but not Mg²⁺. Full length recombinant WSTF proteins isolated from insect cells phosphorylated free H2A.X protein in the presence of Mn²⁺ in the in vitro kinase assays (FIG. 10A). The linear range of the kinase activity was observed from 50-500 uM. On the other hand, no kinase activity was observed in the presence of Mg²⁺. Further increase of Mg²⁺ concentration up to 10 mM did not stimulate the kinase activity. Similar Mn²⁺ dependence was observed with recombinant kinase domain (1-359) (FIG. 10B).

FIGS. 11A-11B show WSTF kinase domain mediated phosphorylation of H2A.X Y142 in vitro. Recombinant WSTF kinase domain phosphorylated WT H2A.X histone proteins in an in vitro kinase assay, while its activity towards a Y142F mutant was much reduced (FIG. 11A). In vitro, WSTF kinase domain activity towards H2A.X was much higher than other core histones (FIG. 11B).

FIG. 12 shows that the mutant (C338A) WSTF kinase domain has a similar protein fold to its WT counterpart. WT and C338A mutant recombinant proteins were diluted to 10 uM and their circular dichroism (CD) spectra (260 to 200 nm) were collected at 25° C. every 5 seconds. Spectra shown were averages of three independent measurements.

FIGS. 13A-13C show the two WSTF kinase domain motifs. The recombinant WSTF kinase domain was partially cleaved into several defined polypeptides (FIG. 13B). The polypeptides were transferred to a PVDF membrane and analyzed by Edman degradation. FIG. 13B shows the sequencing results, which revealed that the recombinant WSTF protein kinase domain consisting of amino acids 1-391 (SEQ ID NO:6) was cleaved into two motifs. Band “a” (SEQ ID NO:8, shown in the bottom of FIG. 13B), represents the WSTF kinase domain plus an N-terminal linker sequence and a C-terminal affinity tag. The N-terminal WSTF kinase motif, band “b”, consists of amino acid residues G1-C344 of SEQ ID NO:8. The C-terminal motif, band “d”, consisting of residues K213-H489 of SEQ ID NO: 8 and band “e” consisting of amino acid residues K213-H477 of SEQ ID NO:8, starts from residue K207 of the WSTF kinase domain (SEQ ID NO:6) (band “e” is a further truncated form of band “d”). Band “c” is the co-purified GST affinity tag. The start of N-terminal motif and C-terminal motif of the WSTF kinase domain are underlined in the WSTF kinase domain amino acid sequence of SEQ ID NO:8 shown in FIG. 13B. FIG. 13C is an alignment of multiple putative kinase regions of WSTF proteins (SEQ ID NOS: 10 to 18) from various vertebrates and invertebrates. Residues conserved in at least 50% of the sequences are shown on black background, and invariant residues are shown on grey background. Sequence abbreviations: hs, human (SEQ ID NO: 9); mm, mouse (SEQ ID NO: 10); gg, chicken (SEQ ID NO: 11); xl, xenopus laevis (SEQ ID NO: 12); dr, zebrafish (SEQ ID NO: 13); cs, ciona savignyi (SEQ ID NO: 14); ac, aplysia californica (SEQ ID NO: 15); lg, lottia gigantea (SEQ ID NO: 16); and pl, paracentrotus lividus (SEQ ID NO: 17).

### DETAILED DESCRIPTION OF THE INVENTION

A first aspect of the present invention relates to an antibody or antigen-binding fragment thereof that selectively binds to a phosphorylated tyrosine residue in an SQEY tyrosine phosphorylation motif sequence (SEQ ID NO: 2). The novel SQEY phosphorylation motif sequence was identified in the histone 2A variant, H2A.X. The full-length amino acid sequence of human H2A.X is provided below as SEQ ID NO:1.

```
Ser Gly Arg Gly Lys Thr Gly Gly Lys Ala Arg Ala Lys Ala Lys
1 5 10 15
Ser Arg Ser Ser Arg Ala Gly Leu Gln Phe Pro Val Gly Arg Val His
                                  20 25 30
Arg Leu Leu Arg Lys Gly His Tyr Ala Glu Arg Val Gly Ala Gly Ala
                          35 40 45
Pro Val Tyr Leu Ala Ala Val Leu Glu Tyr Leu Thr Ala Glu Ile Leu
                  50 55 60
Glu Leu Ala Gly Asn Ala Ala Arg Asp Asn Lys Lys Thr Arg Ile Ile
        65 70 75
Pro Arg His Leu Gln Leu Ala Ile Arg Asn Asp Glu Glu Leu Asn Lys
80 85 90 95
Leu Leu Gly Gly Val Thr Ile Ala Gln Gly Gly Val Leu Pro Asn Ile
                                100 105 110
Gln Ala Val Leu Leu Pro Lys Lys Thr Ser Ala Thr Val Gly Pro Lys
                        115 120 125
Ala Pro Ser Gly Gly Lys Lys Ala Thr Gln Ala Ser Gln Glu Tyr
                130 135 140
```

In a preferred embodiment of the present invention, the antibody or antigen binding fragment thereof selectively binds to the phosphorylated tyrosine residue of the SQEY motif at amino acid position 142 of SEQ ID NO:1.

A second aspect of the present invention relates to an antibody or antigen-binding fragment thereof that selectively binds to the phosphorylated serine residue closest to the glutamine residue in an KENSSQ phosphorylation motif sequence (SEQ ID NO:4). The novel KENSSQ phosphorylation motif sequence was identified in the N-terminal region of the WSTF protein. The full-length amino acid sequence of the human WSTF protein is provided below as SEQ ID NO:3.

```
Met Ala Pro Leu Leu Gly Arg Lys Pro Phe Pro Leu Val Lys Pro Leu
1 5 10 15
Pro Gly Glu Glu Pro Leu Phe Thr Ile Pro His Thr Gln Glu Ala Phe
                        20 25 30
Arg Thr Arg Glu Glu Tyr Glu Ala Arg Leu Glu Arg Tyr Ser Glu Arg
                35 40 45
Ile Trp Thr Cys Lys Ser Thr Gly Ser Ser Gln Leu Thr His Lys Glu
        50 55 60
Ala Trp Glu Glu Glu Gln Glu Val Ala Glu Leu Leu Lys Glu Glu Phe
65 70 75 80
Pro Ala Trp Tyr Glu Lys Leu Val Leu Glu Met Val His His Asn Thr
                                85 90 95
Ala Ser Leu Glu Lys Leu Val Asp Thr Ala Trp Leu Glu Ile Met Thr
                        100 105 110
Lys Tyr Ala Val Gly Glu Glu Cys Asp Phe Glu Val Gly Lys Glu Lys
                115 120 125
Met Leu Lys Val Lys Ile Val Lys Ile His Pro Leu Glu Lys Val Asp
        130 135 140
Glu Glu Ala Thr Glu Lys Lys Ser Asp Gly Ala Cys Asp Ser Pro Ser
145 150 155 160
Ser Asp Lys Glu Asn Ser Ser Gln Ile Ala Gln Asp His Gln Lys Lys
                                165 170 175
Glu Thr Val Val Lys Glu Asp Glu Gly Arg Arg Glu Ser Ile Asn Asp
                        180 185 190
Arg Ala Arg Arg Ser Pro Arg Lys Leu Pro Thr Ser Leu Lys Lys Gly
                195 200 205
Glu Arg Lys Trp Ala Pro Pro Lys Phe Leu Pro His Lys Tyr Asp Val
        210 215 220
Lys Leu Gln Asn Glu Asp Lys Ile Ile Ser Asn Val Pro Ala Asp Ser
225 230 235 240
Leu Ile Arg Thr Glu Arg Pro Pro Asn Lys Glu Ile Val Arg Tyr Phe
                                245 250 255
Ile Arg His Asn Ala Leu Arg Ala Gly Thr Gly Glu Asn Ala Pro Trp
                        260 265 270
Val Val Glu Asp Glu Leu Val Lys Lys Tyr Ser Leu Pro Ser Lys Phe
                275 280 285
Ser Asp Phe Leu Leu Asp Pro Tyr Lys Tyr Met Thr Leu Asn Pro Ser
        290 295 300
Thr Lys Arg Lys Asn Thr Gly Ser Pro Asp Arg Lys Pro Ser Lys Lys
305 310 315 320
Ser Lys Thr Asp Asn Ser Ser Leu Ser Ser Pro Leu Asn Pro Lys Leu
                                325 330 335
Trp Cys His Val His Leu Lys Lys Ser Leu Ser Gly Ser Pro Leu Lys
                        340 345 350
Val Lys Asn Ser Lys Asn Ser Lys Ser Pro Glu Glu His Leu Glu Glu
                355 360 365
Met Met Lys Met Met Ser Pro Asn Lys Leu His Thr Asn Phe His Ile
        370 375 380
Pro Lys Lys Gly Pro Pro Ala Lys Lys Pro Gly Lys His Ser Asp Lys
385 390 395 400
Pro Leu Lys Ala Lys Gly Arg Ser Lys Gly Ile Leu Asn Gly Gln Lys
                                405 410 415
Ser Thr Gly Asn Ser Lys Ser Pro Lys Lys Gly Leu Lys Thr Pro Lys
                        420 425 430
Thr Lys Met Lys Gln Met Thr Leu Leu Asp Met Ala Lys Gly Thr Gln
                435 440 445
Lys Met Thr Arg Ala Pro Arg Asn Ser Gly Gly Thr Pro Arg Thr Ser
        450 455 460
Ser Lys Pro His Lys His Leu Pro Pro Ala Ala Leu His Leu Ile Ala
465 470 475 480
Tyr Tyr Lys Glu Asn Lys Asp Arg Glu Asp Lys Arg Ser Ala Leu Ser
                                485 490 495
Cys Val Ile Ser Lys Thr Ala Arg Leu Leu Ser Ser Glu Asp Arg Ala
                        500 505 510
Arg Leu Pro Glu Glu Leu Arg Ser Leu Val Gln Lys Arg Tyr Glu Leu
                515 520 525
Leu Glu His Lys Lys Arg Trp Ala Ser Met Ser Glu Glu Gln Arg Lys
        530 535 540
Glu Tyr Leu Lys Lys Lys Arg Glu Glu Leu Lys Lys Lys Leu Lys Glu
545 550 555 560
Lys Ala Lys Glu Arg Arg Glu Lys Glu Met Leu Glu Arg Leu Glu Lys
                                565 570 575
Gln Lys Arg Tyr Glu Asp Gln Glu Leu Thr Gly Lys Asn Leu Pro Ala
                        580 585 590
Phe Arg Leu Val Asp Thr Pro Glu Gly Leu Pro Asn Thr Leu Phe Gly
                595 600 605
Asp Val Ala Met Val Val Glu Phe Leu Ser Cys Tyr Ser Gly Leu Leu
        610 615 620
Leu Pro Asp Ala Gln Tyr Pro Ile Thr Ala Val Ser Leu Met Glu Ala
625 630 635 640
Leu Ser Ala Asp Lys Gly Gly Phe Leu Tyr Leu Asn Arg Val Leu Val
                                645 650 655
Ile Leu Leu Gln Thr Leu Leu Gln Asp Glu Ile Ala Glu Asp Tyr Gly
                        660 665 670
Glu Leu Gly Met Lys Leu Ser Glu Ile Pro Leu Thr Leu His Ser Val
                675 680 685
Ser Glu Leu Val Arg Leu Cys Leu Arg Arg Ser Asp Val Gln Glu Glu
        690 695 700
Ser Glu Gly Ser Asp Thr Asp Asp Asn Lys Asp Ser Ala Ala Phe Glu
705 710 715 720
Asp Asn Glu Val Gln Asp Glu Phe Leu Glu Lys Leu Glu Thr Ser Glu
                                725 730 735
Phe Phe Glu Leu Thr Ser Glu Glu Lys Leu Gln Ile Leu Thr Ala Leu
                        740 745 750
Cys His Arg Ile Leu Met Thr Tyr Ser Val Gln Asp His Met Glu Thr
                755 760 765
Arg Gln Gln Met Ser Ala Glu Leu Trp Lys Glu Arg Leu Ala Val Leu
        770 775 780
Lys Glu Glu Asn Asp Lys Lys Arg Ala Glu Lys Gln Lys Arg Lys Glu
785 790 795 800
Met Glu Ala Lys Asn Lys Glu Asn Gly Lys Val Glu Asn Gly Leu Gly
                                805 810 815
Lys Thr Asp Arg Lys Lys Glu Ile Val Lys Phe Glu Pro Gln Val Asp
                        820 825 830
Thr Glu Ala Glu Asp Met Ile Ser Ala Val Lys Ser Arg Arg Leu Leu
                835 840 845
Ala Ile Gln Ala Lys Lys Glu Arg Glu Ile Gln Glu Arg Glu Met Lys
        850 855 860
Val Lys Leu Glu Arg Gln Ala Glu Glu Glu Arg Ile Arg Lys His Lys
865 870 875 880
Ala Ala Ala Glu Lys Ala Phe Gln Glu Gly Ile Ala Lys Ala Lys Leu
                                885 890 895
Val Met Arg Arg Thr Pro Ile Gly Thr Asp Arg Asn His Asn Arg Tyr
                        900 905 910
Trp Leu Phe Ser Asp Glu Val Pro Gly Leu Phe Ile Glu Lys Gly Trp
                915 920 925
Val His Asp Ser Ile Asp Tyr Arg Phe Asn His His Cys Lys Asp His
        930 935 940
Thr Val Ser Gly Asp Glu Asp Tyr Cys Pro Arg Ser Lys Lys Ala Asn
945 950 955 960
Leu Gly Lys Asn Ala Ser Met Asn Thr Gln His Gly Thr Ala Thr Glu
                                965 970 975
Val Ala Val Glu Thr Thr Thr Pro Lys Gln Gly Gln Asn Leu Trp Phe
                        980 985 990
Leu Cys Asp Ser Gln Lys Glu Leu Asp Glu Leu Leu Asn Cys Leu His
                995 1000 1005
Pro Gln Gly Ile Arg Glu Ser Gln Leu Lys Glu Arg Leu Glu Lys
        1010 1015 1020
Arg Tyr Gln Asp Ile Ile His Ser Ile His Leu Ala Arg Lys Pro
        1025 1030 1035
Asn Leu Gly Leu Lys Ser Cys Asp Gly Asn Gln Glu Leu Leu Asn
        1040 1045 1050
Phe Leu Arg Ser Asp Leu Ile Glu Val Ala Thr Arg Leu Gln Lys
        1055 1060 1065
Gly Gly Leu Gly Tyr Val Glu Glu Thr Ser Glu Phe Glu Ala Arg
        1070 1075 1080
Val Ile Ser Leu Glu Lys Leu Lys Asp Phe Gly Glu Cys Val Ile
        1085 1090 1095
Ala Leu Gln Ala Ser Val Ile Lys Lys Phe Leu Gln Gly Phe Met
        1100 1105 1110
Ala Pro Lys Gln Lys Arg Arg Lys Leu Gln Ser Glu Asp Ser Ala
        1115 1120 1125
Lys Thr Glu Glu Val Asp Glu Glu Lys Lys Met Val Glu Glu Ala
        1130 1135 1140
Lys Val Ala Ser Ala Leu Glu Lys Trp Lys Thr Ala Ile Arg Glu
        1145 1150 1155
Ala Gln Thr Phe Ser Arg Met His Val Leu Leu Gly Met Leu Asp
        1160 1165 1170
Ala Cys Ile Lys Trp Asp Met Ser Ala Glu Asn Ala Arg Cys Lys
        1175 1180 1185
Val Cys Arg Lys Lys Gly Glu Asp Asp Lys Leu Ile Leu Cys Asp
        1190 1195 1200
Glu Cys Asn Lys Ala Phe His Leu Phe Cys Leu Arg Pro Ala Leu
        1205 1210 1215
Tyr Glu Val Pro Asp Gly Glu Trp Gln Cys Pro Ala Cys Gln Pro
        1220 1225 1230
Ala Thr Ala Arg Arg Asn Ser Arg Gly Arg Asn Tyr Thr Glu Glu
        1235 1240 1245
Ser Ala Ser Glu Asp Ser Glu Asp Asp Glu Ser Asp Glu Glu Glu
        1250 1255 1260
Glu Glu Glu Glu Glu Glu Glu Glu Glu Glu Asp Tyr Glu Val Ala
        1265 1270 1275
Gly Leu Arg Leu Arg Pro Arg Lys Thr Ile Arg Gly Lys His Ser
        1280 1285 1290
Val Ile Pro Pro Ala Ala Arg Ser Gly Arg Arg Pro Gly Lys Lys
        1295 1300 1305
Pro His Ser Thr Arg Arg Ser Gln Pro Lys Ala Pro Pro Val Asp
        1310 1315 1320
Asp Ala Glu Val Asp Glu Leu Val Leu Gln Thr Lys Arg Ser Ser
        1325 1330 1335
Arg Arg Gln Ser Leu Glu Leu Gln Lys Cys Glu Glu Ile Leu His
        1340 1345 1350
Lys Ile Val Lys Tyr Arg Phe Ser Trp Pro Phe Arg Glu Pro Val
        1355 1360 1365
Thr Arg Asp Glu Ala Glu Asp Tyr Tyr Asp Val Ile Thr His Pro
        1370 1375 1380
Met Asp Phe Gln Thr Val Gln Asn Lys Cys Ser Cys Gly Ser Tyr
        1385 1390 1395
Arg Ser Val Gln Glu Phe Leu Thr Asp Met Lys Gln Val Phe Thr
        1400 1405 1410
Asn Ala Glu Val Tyr Asn Cys Arg Gly Ser His Val Leu Ser Cys
        1415 1420 1425
Met Val Lys Thr Glu Gln Cys Leu Val Ala Leu Leu His Lys His
        1430 1435 1440
Leu Pro Gly His Pro Tyr Val Arg Arg Lys Arg Lys Lys Phe Pro
        1445 1450 1455
Asp Arg Leu Ala Glu Asp Glu Gly Asp Ser Glu Pro Glu Ala Val
        1460 1465 1470
Gly Gln Ser Arg Gly Arg Arg Gln Lys Lys
        1475 1480
```

In a preferred embodiment of the present invention, the antibody or antigen binding fragment thereof selectively binds to the phosphorylated serine closest to the glutamine residue in the KENSSQ phosphorylation motif at amino acid position 167 of SEQ ID NO: 3.

The antibodies of the present invention can be monoclonal or polyclonal.

Procedures for raising polyclonal antibodies are well known in the art. Typically, such antibodies are raised by administering a peptide containing the epitope of interest, i.e., a tyrosine phosphorylated SQEY peptide or serine phosphorylated KENSSQ peptide, subcutaneously to New Zealand white rabbits which have first been bled to obtain pre-immune serum. The antigens can be injected at a total volume of 100 μl per site at six different sites. Injected material will contain synthetic surfactant adjuvant pluronic polyols, or pulverized acrylamide gel containing the protein or polypeptide after SDS-polyacrylamide gel electrophoresis. The rabbits are bled approximately every two weeks after the first injection and periodically boosted with the same antigen three times every six weeks. A sample of serum is collected 10 days after each boost and polyclonal antibodies are recovered by affinity chromatography using the corresponding antigen to capture the antibody. This and other procedures for raising polyclonal antibodies are disclosed in ANTIBODIES: A LABORATORY MANUAL (Harlow et al. eds., 1988), which is hereby incorporated by reference in its entirety.

A monoclonal antibody is obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See U.S. Pat. No. 4,816,567 to Cabilly et al., and Morrison et al., “Chimeric Human Antibody Molecules Mouse Antigen-Binding Domains with Human Constant Region Domains,” Proc. Natl. Acad. Sci. USA, 81:6851-6855 (1984) which are hereby incorporated by reference in their entirety).

Monoclonal antibodies may be prepared using hybridoma methods, such as those described by Kohler et al., “Continuous Cultures of Fused Cells Secreting Antibody of Predefined Specificity,” Nature 256:495-7 (1975) or ANTIBODIES: A LABORATORY MANUAL (Harlow et al. eds., 1988), which are hereby incorporated by reference in their entirety. In a hybridoma method, a mouse or other appropriate host animal, is immunized with an immunizing agent to elicit lymphocytes to produce antibodies that will specifically bind to the immunizing agent. Alternatively, the lymphocytes may be immunized in vitro. In accordance with the present invention, the immunizing agent comprises a tyrosine phosphorylated SQEY peptide sequence or a serine phosphorylated KENSSQ sequence, where the serine closest to the glutamine residue is phosphorylated.

In addition to the traditional approaches for generating monoclonal antibodies, which depend on the availability of purified protein or peptide for use as the immunogen, more recently developed DNA based immunizations are also suitable for generating the antibodies of the present invention. In this approach, DNA-based immunization can be used, where DNA encoding the SQEY or KENSSQ motifs are expressed as a fusion protein with human IgG1 and injected into the host animal according to methods known in the art (e.g., Kilpatrick et al., “Gene Gun Delivered DNA-Based Immunizations Mediate Rapid Production of Murine Monoclonal Antibodies to the Flt-3 Receptor,” Hybridoma 17(6):569-76 (1998) and Kilpatrick et al., “High-Affinity Monoclonal Antibodies to PED/PEA-15 Generated Using 5 Micrograms of DNA,” Hybridoma 19(4):297-302 (2000), which are hereby incorporated by reference in their entirety. Alternatively, the nucleic acid sequences encoding the SQEY or KENSSQ motifs be can expressed in a baculovirus expression system. The advantages to this system include ease of generation, high levels of expression, and post-translational modifications that are highly similar to those seen in mammalian systems.

Generally, peripheral blood lymphocytes are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (JAMES W. GODING, MONOCLONAL ANTIBODIES: PRINCIPLES AND PRACTICE (Academic Press 1986), which is hereby incorporated by reference in its entirety). Immortalized cell lines are usually transformed mammalian cells, including myeloma cells of rodent, bovine, equine, and human origin. The hybridoma cells are cultured in a suitable culture medium that preferably contains one or more substances that inhibit the growth or survival of the unfused, immortalized cells. Preferred immortalized cell lines (e.g., murine myeloma lines) are those that fuse efficiently and support stable high level expression of antibody by the selected antibody-producing cells. Human myeloma and mouse-human heteromyeloma cell lines have also been described for the production of human monoclonal antibodies (Kozbor et al., “A Human Hybrid Myeloma for Production of Human Monoclonal Antibodies,” J. Immunol. 133:3001-5 (1984) and MONOCLONAL ANTIBODY PRODUCTION TECHNIQUES AND APPLICATIONS (L. B. Shook ed., 1987), which are hereby incorporated by reference in their entirety). The culture medium in which the hybridoma cells are cultured can be assayed for the presence of monoclonal antibodies directed against the phosphotyrosine of the SQEY motif or the phosphoserine of the KENSSQ motif. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA), or chemiluminescence assays. Such techniques and assays are known in the art, and are fully described in ANTIBODIES: A LABORATORY MANUAL (Harlow et al. eds., 1988), which is hereby incorporated by reference in its entirety.

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies of the present invention may also be made by recombinant DNA methods, such as those described in U.S. Pat. No. 4,816,567 to Cabilly et al, which is hereby incorporated by reference in its entirety. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells, using the appropriate vectors described herein. The DNA also may be modified, for example, by substituting the coding sequence for human heavy and light chain constant domains in place of the homologous murine sequences or by covalently joining to the immunoglobulin coding sequence all or part of the coding sequence for a non-immunoglobulin polypeptide (See U.S. Pat. No. 4,816,567 to Cabilly et al, which is hereby incorporated by reference in its entirety). Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for either the phosphotyrosine of the SQEY motif or the phosphoserine of the KENSSQ motif and another antigen-combining site having specificity for a different antigen.

The antibodies of the present invention may be whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain typically has a variable domain at one end (V(L)) and a constant domain at its other end. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2.

The variable regions of the heavy and light chains form a cleft which comprises the antigen binding domain of antibody. Antibodies of the present invention can be mono-, bi-, or multivalent (i.e., having one, two, or multiple antigen binding domains).

In addition to whole antibodies, the present invention encompasses chimeric antibodies, hybrid antibodies, and fragments, such as scFv, sFv, F(ab′)₂, Fab′, Fab and the like, including hybrid fragments. Thus, fragments of the antibodies that retain phosphotyrosine or phosphoserine binding activity are included within the meaning of antibody or antigen binding fragment thereof. Such antibodies and fragments can be made and screened for specificity and activity by techniques known in the art (see ANTIBODIES: A LABORATORY MANUAL (Harlow et al. eds., 1988), which is hereby incorporated by reference in its entirety).

Monovalent antibodies can be generated by in vitro digestion of whole antibodies to produce fragments, i.e., Fab fragments, using routine techniques known in the art. For instance, digestion can be performed using papain as described in WO94/29348 to Landon, and ANTIBODIES: A LABORATORY MANUAL (Harlow et al. eds., 1988), which are hereby incorporated by reference in their entirety. Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen. Methods for generating stable monovalent antibody fragments for therapeutic utility are further described in WO/2005063816 to Huang et al., which is hereby incorporated by reference in its entirety.

The Fab fragments produced by antibody digestion contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity. Immunoreactive paratopes of the antibody, optionally, are synthesized directly. An immunoreactive fragment is defined as an amino acid sequence of at least about two to five consecutive amino acids derived from the antibody amino acid sequence.

The antibodies of the present invention can be generated in a non-human species and “humanized” for administration in humans. Humanized forms of non-human (e.g., murine) antibodies are chimeric immunoglobulins, immunoglobulin chains or fragments thereof which contain minimal sequence derived from non-human immunoglobulin. Humanized antibodies include human immunoglobulins in which residues of the complementary determining region (CDR) are replaced by residues from a CDR of a non-human species such as mouse, rat, or rabbit having the desired specificity, affinity, and capacity. In some instances, Fv framework residues of the human immunoglobulin are replaced by corresponding non-human residues. Methods for humanizing non-human antibodies are well known in the art as described in U.S. Pat. No. 4,816,567 to Cabilly et al.; Jones et al., “Replacing the Complementarity-Determining Regions in a Human Antibody with those From a Mouse,” Nature 321:522-525 (1986); Riechmann et al., “Reshaping Human Antibodies for Therapy,” Nature 332:323-327 (1988); and Verhoeyen et al., “Reshaping Human Antibodies: Grafting an Antilysozyme Activity,” Science 239:1534-1536 (1988), which are hereby incorporated by reference in their entirety.

Another aspect of the present invention relates to applicants' discovery of a novel kinase domain within the N-terminal region of the WSTF protein. As described herein, WSTF is a component of the WICH ATP-dependent chromatin remodeling complex. The full-length amino acid sequence of the human WSTF protein is set forth supra in SEQ ID NO:3. The novel kinase domain of the WSTF is found within amino acids 1-391 of the full length WSTF protein sequence of SEQ ID NO:3 and comprises two motifs, a conserved N-terminal WAC domain and a more divergent C-terminal domain. These two motifs are linked by a long loop region where the ATM phosphorylation site resides (S167 in KENSSQ). Accordingly, another aspect of the present invention relates to an isolated polypeptide having an amino acid sequence of the novel WSTF kinase domain as set forth below as SEQ ID NO:6.

```
Met Ala Pro Leu Leu Gly Arg Lys Pro Phe Pro Leu Val Lys Pro Leu
1 5 10 15
Pro Gly Glu Glu Pro Leu Phe Thr Ile Pro His Thr Gln Glu Ala Phe
                        20 25 30
Arg Thr Arg Glu Glu Tyr Glu Ala Arg Leu Glu Arg Tyr Ser Glu Arg
                35 40 45
Ile Trp Thr Cys Lys Ser Thr Gly Ser Ser Gln Leu Thr His Lys Glu
        50 55 60
Ala Trp Glu Glu Glu Gln Glu Val Ala Glu Leu Leu Lys Glu Glu Phe
65 70 75 80
Pro Ala Trp Tyr Glu Lys Leu Val Leu Glu Met Val His His Asn Thr
                                85 90 95
Ala Ser Leu Glu Lys Leu Val Asp Thr Ala Trp Leu Glu Ile Met Thr
                        100 105 110
Lys Tyr Ala Val Gly Glu Glu Cys Asp Phe Glu Val Gly Lys Glu Lys
                115 120 125
Met Leu Lys Val Lys Ile Val Lys Ile His Pro Leu Glu Lys Val Asp
        130 135 140
Glu Glu Ala Thr Glu Lys Lys Ser Asp Gly Ala Cys Asp Ser Pro Ser
145 150 155 160
Ser Asp Lys Glu Asn Ser Ser Gln Ile Ala Gln Asp His Gln Lys Lys
                                165 170 175
Glu Thr Val Val Lys Glu Asp Glu Gly Arg Arg Glu Ser Ile Asn Asp
                        180 185 190
Arg Ala Arg Arg Ser Pro Arg Lys Leu Pro Thr Ser Leu Lys Lys Gly
                195 200 205
Glu Arg Lys Trp Ala Pro Pro Lys Phe Leu Pro His Lys Tyr Asp Val
        210 215 220
Lys Leu Gln Asn Glu Asp Lys Ile Ile Ser Asn Val Pro Ala Asp Ser
225 230 235 240
Leu Ile Arg Thr Glu Arg Pro Pro Asn Lys Glu Ile Val Arg Tyr Phe
                                245 250 255
Ile Arg His Asn Ala Leu Arg Ala Gly Thr Gly Glu Asn Ala Pro Trp
                        260 265 270
Val Val Glu Asp Glu Leu Val Lys Lys Tyr Ser Leu Pro Ser Lys Phe
                275 280 285
Ser Asp Phe Leu Leu Asp Pro Tyr Lys Tyr Met Thr Leu Asn Pro Ser
        290 295 300
Thr Lys Arg Lys Asn Thr Gly Ser Pro Asp Arg Lys Pro Ser Lys Lys
305 310 315 320
Ser Lys Thr Asp Asn Ser Ser Leu Ser Ser Pro Leu Asn Pro Lys Leu
                                325 330 335
Trp Cys His Val His Leu Lys Lys Ser Leu Ser Gly Ser Pro Leu Lys
                        340 345 350
Val Lys Asn Ser Lys Asn Ser Lys Ser Pro Glu Glu His Leu Glu Glu
                355 360 365
Met Met Lys Met Met Ser Pro Asn Lys Leu His Thr Asn Phe His Ile
        370 375 380
Pro Lys Lys Gly Pro Pro Ala
385 390
```

The kinase domain of the human WSTF protein of SEQ ID NO:6 is encoded by the nucleic acid sequence set forth below as SEQ ID NO:5.

| | |
|---|---|
| atggcgccgc tcctgggccg caagcccttc ccgctggtga agccgttgcc cggagaggag | 60 |
| ccgctcttca ccatcccgca cactcaggag gccttccgca cccgggaaga gtatgaagcc | 120 |
| cgcttggaaa ggtacagtga gcgcatttgg acgtgcaaga gtactggaag cagtcagcta | 180 |
| acacacaagg aagcctggga ggaagaacag gaagttgctg agcttttgaa ggaggagttt | 240 |
| cctgcctggt atgagaagct tgttctggaa atggttcacc ataacacagc ctccttagag | 300 |
| aagttagtag atactgcttg gttggagatc atgaccaaat atgctgtggg agaagagtgt | 360 |
| gacttcgagg ttgggaagga gaaaatgctc aaggtgaaga ttgtgaagat tcatcctttg | 420 |
| gagaaagtgg atgaagaggc cactgagaag aaatctgatg gtgcctgtga ttctccatca | 480 |
| agtgacaaag agaactccag tcagattgct caggaccatc agaagaagga gacagttgtg | 540 |
| aaagaggatg aaggaaggag agagagtatt aatgacagag cacgtagatc gccacgaaaa | 600 |
| cttcctactt cattaaaaaa aggagaaagg aaatgggctc ctccaaaatt tctgcctcac | 660 |
| aaatatgatg tgaaactaca aaatgaagat aagatcatca gtaacgtgcc agcagacagc | 720 |
| ttgattcgta cagagcgccc accaaataag gagatagttc gatactttat acggcataat | 780 |
| gcattacgag ctggtactgg tgaaaatgca ccttgggtcg tagaagatga attggtgaag | 840 |
| aaatactctc tgcccagcaa gttcagtgac tttttacttg atccatacaa gtatatgact | 900 |
| ctcaaccctt ctactaagag gaagaatact ggatccccag acaggaagcc ctcaaagaaa | 960 |
| tccaagacag acaactcttc tcttagttca ccactaaatc ctaagttatg gtgtcacgta | 1020 |
| cacttgaaga agtcattgag tggctcgcca ctcaaagtga agaactcaaa gaattccaaa | 1080 |
| tctcctgaag aacatctaga agaaatgatg aagatgatgt cgcccaataa gctgcacact | 1140 |
| aactttcaca ttcctaaaaa aggcccacct gcc | 1173 |

Accordingly, the present invention also relates to an isolated nucleic acid molecule having a nucleotide sequence of SEQ ID NO:5. In a preferred embodiment, the isolated nucleic acid molecule of the present invention has at least 80% nucleic acid sequence identity to the nucleotide sequence of SEQ ID NO: 5. More preferably, the isolated nucleic acid molecule of the present invention has at least 90% nucleic acid sequence identity to the nucleotide sequence of SEQ ID NO:5. Most preferably, the isolated nucleic acid molecule of the present invention has at least 95% nucleic acid sequence identity to the nucleotide sequence of SEQ ID NO:5.

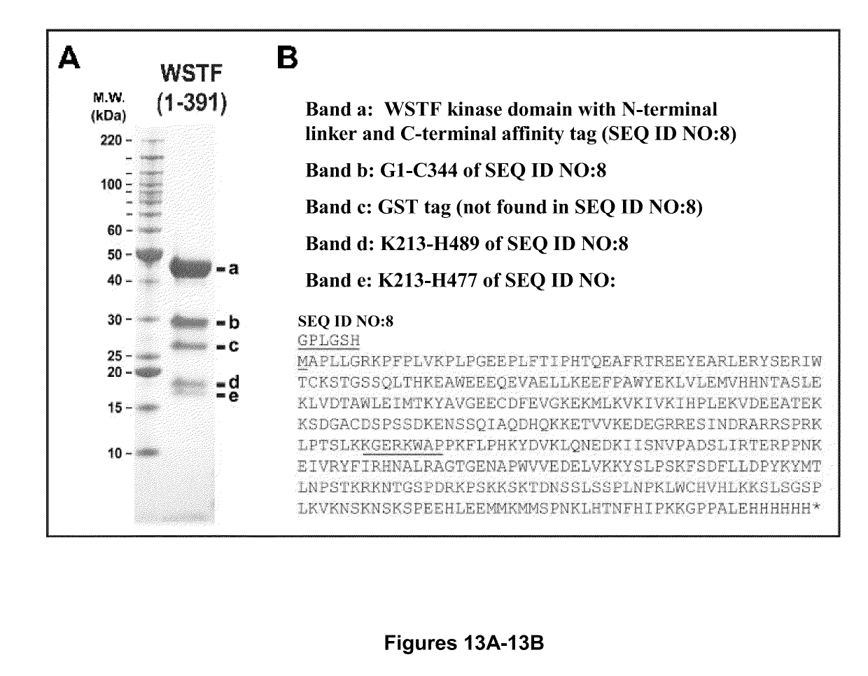

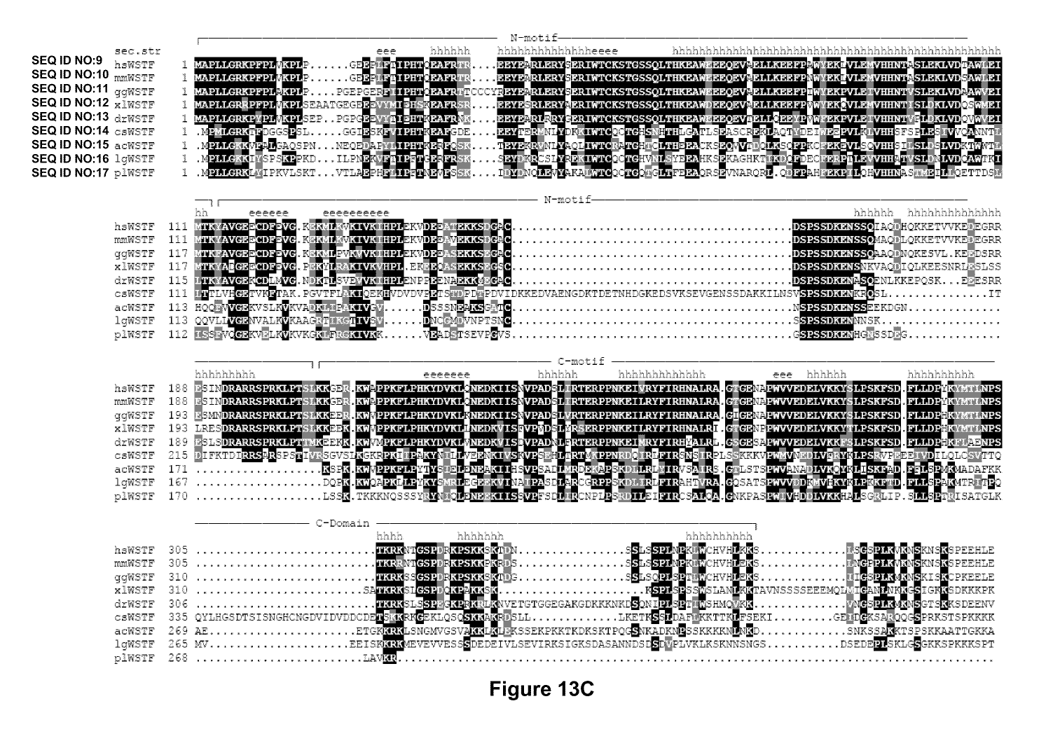

In an alternative embodiment, the isolated polypeptide molecule of the present invention is derived from a WSTF kinase domain consensus sequence. A WSTF kinase domain consensus sequence was generated by aligning the putative kinase regions of WSTF proteins from various vertebrates and invertebrates as shown in FIG. 13. The resulting amino acid sequence of the WSTF consensus sequence is set forth below as SEQ ID NO: 7, where X is any amino acid.

```
Xaa Xaa Xaa Xaa Xaa Xaa Xaa Lys Xaa Xaa Xaa Xaa Xaa Xaa Xaa Xaa
1 5 10 15
Xaa Xaa Xaa Xaa Xaa Xaa Phe Xaa Xaa Xaa Xaa Xaa Xaa Xaa Xaa Xaa
                        20 25 30
Xaa Xaa Xaa Xaa Glu Glu Tyr Glu Xaa Arg Leu Glu Arg Tyr Xaa Glu
                35 40 45
Arg Ile Trp Thr Cys Lys Ser Thr Gly Ser Ser Gln Leu Thr His Xaa
        50 55 60
Xaa Xaa Xaa Xaa Xaa Glu Xaa Glu Val Xaa Glu Leu Leu Lys Glu Glu
65 70 75 80
Phe Pro Xaa Trp Xaa Glu Lys Leu Val Leu Glu Xaa Val His His Asn
                                85 90 95
Thr Xaa Ser Leu Glu Lys Leu Val Asp Xaa Ala Trp Xaa Glu Ile Xaa
                        100 105 110
Thr Lys Xaa Ala Val Gly Glu Xaa Cys Asp Phe Xaa Val Gly Xaa Xaa
                115 120 125
Lys Xaa Leu Xaa Xaa Lys Ile Val Lys Xaa His Pro Leu Xaa Xaa Xaa
        130 135 140
Xaa Xaa Xaa Xaa Xaa Xaa Xaa Xaa Xaa Xaa Xaa Xaa Xaa Xaa Xaa Xaa
145 150 155 160
Xaa Xaa Lys Glu Asn Ser Ser Gln Xaa Xaa Xaa Xaa Xaa Xaa Xaa Xaa
                                165 170 175
Xaa Xaa Xaa Xaa Xaa Xaa Xaa Xaa Xaa Xaa Xaa Xaa Xaa Xaa Xaa Xaa
                        180 185 190
Xaa Xaa Xaa Xaa Xaa Xaa Xaa Xaa Xaa Xaa Xaa Xaa Xaa Lys Lys Xaa
                195 200 205
Xaa Xaa Lys Trp Xaa Pro Pro Lys Phe Leu Pro His Lys Tyr Asp Val
        210 215 220
Lys Leu Xaa Asn Glu Asp Lys Ile Ile Ser Xaa Val Pro Ala Asp Xaa
225 230 235 240
Leu Xaa Arg Thr Glu Arg Pro Pro Asn Lys Glu Ile Xaa Arg Tyr Phe
                                245 250 255
Ile Arg His Asn Ala Leu Arg Ala Gly Xaa Gly Glu Xaa Xaa Pro Trp
                        260 265 270
Val Val Glu Asp Glu Leu Val Lys Lys Tyr Xaa Leu Pro Ser Lys Phe
                275 280 285
Ser Asp Phe Leu Leu Asp Pro Xaa Lys Xaa Xaa Xaa Xaa Asn Pro Ser
        290 295 300
Thr Lys Arg Lys Xaa Xaa Gly Ser Pro Xaa Xaa Lys Pro Ser Lys Lys
305 310 315 320
Xaa Lys Xaa Xaa Xaa Xaa Ser Xaa Xaa Xaa Xaa Xaa Xaa Xaa Xaa Xaa
                                325 330 335
Trp Xaa Xaa Xaa Xaa Xaa Xaa Xaa Xaa Xaa Xaa Xaa Xaa Xaa Xaa Xaa
                        340 345 350
Xaa Xaa Xaa Xaa Xaa Xaa Xaa Xaa Xaa Xaa Xaa Xaa Xaa Xaa Xaa
                355 360 365
```

Using standard recombinant cloning technology and techniques well known in the art, and described in the Examples below, the isolated nucleic acid molecules encoding the WSTF kinase domain of the present invention can be inserted into an expression vector and transformed into a host cell to facilitate, inter alias, WSTF kinase domain expression and peptide purification.

An isolated nucleic acid molecule of the present invention is inserted into an expression system to which the molecule is heterologous. The heterologous nucleic acid molecule is inserted into the expression system or vector in proper sense (5′→3′) orientation relative to the promoter and any other 5′ regulatory molecules. The preparation of the nucleic acid constructs can be carried out using standard cloning methods well known in the art as described by SAMBROOK AND RUSSELL, MOLECULAR CLONING: A LABORATORY MANUAL (Cold Springs Laboratory Press, 1989), which is hereby incorporated by reference in its entirety. U.S. Pat. No. 4,237,224 to Cohen and Boyer, which is hereby incorporated by reference in its entirety, also describes the production of expression systems in the form of recombinant plasmids using restriction enzyme cleavage and ligation with DNA ligase.

Suitable expression vectors include those which contain replicon and control sequences that are derived from species compatible with a chosen host cell. For example, if E. coli is used as a host cell, plasmids such as pUC19, pUC18 or pBR322 may be used. As described herein, full length WSTF protein was expressed and purified using a baculovirus system. Therefore, appropriate transfer vectors compatible with insect host cells include, pVL1392, pVL1393, pAcGP67 and pAcSecG2T, which incorporate a secretory signal fused to the desired protein, and pAcGHLT and pAcHLT, which contain GST and 6×His tags (BD Biosciences, Franklin Lakes, N.J.). Other suitable expression vectors are described in SAMBROOK AND RUSSELL, MOLECULAR CLONING: A LABORATORY MANUAL (Cold Springs Laboratory Press, 1989), which is hereby incorporated by reference in its entirety. Many known techniques and protocols for manipulation of nucleic acids, for example in preparation of nucleic acid constructs, mutagenesis, sequencing, introduction of DNA into cells and gene expression, and analysis of proteins, are described in detail in CURRENT PROTOCOLS IN MOLECULAR BIOLOGY (Fred M. Ausubel et al. eds., 1992), which is hereby incorporated by reference in its entirety.

Different genetic signals and processing events control many levels of gene expression (e.g., DNA transcription and messenger RNA (“mRNA”) translation). Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is desirable to use strong promoters to obtain a high level of transcription and, hence, expression and kinase activity. Therefore, depending upon the host system utilized, any one of a number of suitable promoters may also be incorporated into the expression vector carrying the nucleic acid molecules of the present invention. For instance, when using E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. When using insect cells, suitable baculovirus promoters include late promoters, such as 39K protein promoter or basic protein promoter, and very late promoters, such as the p10 and polyhedron promoters. In some cases it may be desirable to use transfer vectors containing multiple baculoviral promoters.

Translation of mRNA in prokaryotes depends upon the presence of the proper prokaryotic signals, which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Host cells suitable for expressing or propagating the nucleic acid construct encoding the WSTF kinase domain include any one of the more commonly available gram negative bacteria. Suitable microorganisms include Pseudomonas aeruginosa, Escherichia coli, Salmonella gastroenteritis (typhimirium), S. typhi, S. enteriditis, Shigella flexneri, S. sonnie, S. dysenteriae, Neisseria gonorrhoeae, N. meningitides, Haemophilus influenzae, H. pleuropneumoniae, Pasteurella haemolytica, P. multilocida, Legionella pneumophila, Treponema pallidum, T. denticola, T. orales, Borrelia burgdorferi, Borrelia spp., Leptospira interrogans, Klebsiella pneumoniae, Proteus vulgaris, P. morganii, P. mirabilis, Rickettsia prowazeki, R. typhi, R. richettsii, Porphyromonas (Bacteriodes) gingivalis, Chlamydia psittaci, C. pneumoniae, C. trachomatis, Campylobacter jejuni, C. intermedis, C. fetus, Helicobacter pylori, Francisella tularenisis, Vibrio cholerae, Vibrio parahaemolyticus, Bordetella pertussis, Burkholderie pseudomallei, Brucella abortus, B. susi, B. melitensis, B. canis, Spirillum minus, Pseudomonas mallei, Aeromonas hydrophile, A. salmonicida, and Yersinia pestis.

In addition to bacteria cells, eukaryotic cells such as mammalian, insect, and yeast systems are also suitable host cells for transfection/transformation of the expression vector carrying an isolated nucleic acid molecule of the present invention. Mammalian cell lines available in the art for expression of a heterologous polypeptide include Chinese hamster ovary cells, HeLa cells, baby hamster kidney cells, COS cells and many others. Suitable insect cell lines include those susceptible to baculoviral infection, including Sf9 and Sf21 cells. Methods for transforming/transfecting host cells with expression vectors are well-known in the art and depend on the host system selected, as described in SAMBROOK AND RUSSELL, MOLECULAR CLONING: A LABORATORY MANUAL (Cold Springs Laboratory Press, 1989), which is hereby incorporated by reference in its entirety. For bacterial cells, suitable techniques may include calcium chloride transformation, electroporation, and transfection using bacteriophage For eukaryotic cells, suitable techniques may include calcium phosphate transfection, DEAE-Dextran, electroporation, liposome-mediated transfection and transduction using retrovirus or other virus, e.g., vaccinia. For insect cells, the transfer vector containing the nucleic acid construct encoding the WSTF kinase domain is co-transfected with baculovirus DNA, such as AcNPV, to facilitate the production of a recombinant virus resulting from homologous recombination between the WSTF construct in the transfer vector and baculovirus DNA. Subsequent recombinant viral infection of Sf cells results in a high rate of recombinant protein production that can be readily purified using standard purification methods known in the art and described in the Examples below.

Another aspect of the present invention relates to a method of identifying cellular DNA damage in a sample. This method involves providing a cell sample and detecting the presence or absence of a phosphorylated tyrosine residue in an SQEY tyrosine phosphorylation motif (SEQ ID NO:2) in the sample. Cellular DNA damage in the cell sample is identified based on detecting the presence or absence of tyrosine phosphorylation.

In a preferred embodiment of the invention, the presence or absence of a phosphotyrosine residue in the SQEY motif at amino acid position 142 of SEQ ID NO:1 is detected.

The presence or absence of the phosphotyrosine residue, in accordance with this aspect of the present invention, can be detected using the antibody or antigen-binding fragment thereof of the present invention that selectively binds to the phosphorylated tyrosine in the SQEY sequence as described supra. Other pan-antibodies capable of binding phosphorylated tyrosine residues are well known in the art and commercially available from a number of companies (e.g., Millipore, Billerica, Ma.; Abcam, Cambridge, Mass.; Santa Cruz, Santa Cruz, Calif.). In a preferred embodiment, the antibody or antigen binding fragment used for detecting the phosphotyrosine residue selectively binds to the phosphorylated tyrosine residue of the SQEY at amino acid position 142 of SEQ ID NO:1 as described herein.

Although the use of a phosphotyrosine specific antibody for detecting a phosphorylated tyrosine residue is a convenient method of detection, alternative methods for detecting a phosphorylated tyrosine of the SQEY motif are known in the art and are suitable for use in this aspect of the invention. For example, phosphorylated amino acids can be detected using in situ labeling techniques with [³²P]orthophosphate as described in POST-TRANSLATIONAL PROCESSING: A PRACTICAL APPROACH (Steve Higgens et al., eds., 1999), which is hereby incorporated by reference in its entirety. Alternatively, mass spectrometry based techniques, e.g., liquid chromatography/mass spectrometry/mass spectrometry (LC/MS/MS) can be employed to detect a phosphotyrosine residue in a SQEY motif.

Upon initiation of the cellular response to DNA damage, the tyrosine residue of the SQEY sequence is dephosphorylated. Therefore, in accordance with this aspect of the present invention, detecting an absence of a phosphorylated tyrosine residue in the SQEY motif indicates the presence of cellular DNA damage and detecting the presence of a phosphorylated tyrosine residue in the SQEY motif indicates a lack of cellular DNA damage in the cell sample.

Another aspect of the present invention is directed to a method of identifying cellular DNA damage in a sample. This method involves providing a cell sample and detecting the presence or absence of a phosphorylated serine residue at the serine closest to the glutamine residue in an KENSSQ phosphorylation motif sequence (SEQ ID NO:4). Cellular DNA damage in the cell sample is identified based on detecting the presence or absence of serine phosphorylation.

In a preferred embodiment of the invention, the presence or absence of a phosphorylated serine closest to the glutamine residue of the KENSSQ motif at amino acid position 167 of SEQ ID NO:3 is detected.

Detecting the phosphorylated serine in the KENSSQ motif can be carried out using an antibody or antigen-binding fragment thereof of the present invention that binds to the phosphorylated serine closest to the glutamine residue in an KENSSQ phosphorylation motif sequence (SEQ ID NO:4), as described supra. In a preferred embodiment, an antibody or antigen-binding fragment thereof that selectively binds to the phosphorylated serine closest to the glutamine residue in the KENSSQ motif at amino acid position 167 of SEQ ID NO:3 is used. Non-specific phosphoserine antibodies are also suitable for detecting the phosphorylated serine closest to the glutamine residue in the KENSSQ motif. Any of the alternative methods of detecting phosphorylated amino acid residues that are known in the art, and described supra, are also suitable.

Upon the initiation of DNA damage in a cell, the DNA damage-induced ATM kinase phosphorylates the serine closest to the glutamine residue of the KENSSQ motif in the WSTF amino-terminus. Therefore, in accordance with this aspect of the present invention, detecting the presence of a phosphorylated serine residue at the serine closest to the glutamine residue of the KENSSQ motif indicates the presence of cellular DNA damage, whereas the absence of a phosphorylated serine residue at the serine closest to the glutamine residue of the KENSSQ motif indicates a lack cellular DNA damage.

Another aspect of the present invention relates to a method of screening candidate compounds useful for modulating WSTF kinase activity. This method involves providing a candidate compound and a sample, where the sample contains a WSTF protein or polypeptide having kinase activity, a protein substrate having an SQEY motif sequence, manganese, and ATP. Contacting the candidate compound with the sample and detecting the presence or absence of a phosphorylated tyrosine residue in the SQEY tyrosine phosphorylation motif (SEQ ID NO:2) identifies compounds useful for modulating WSTF kinase activity.

In accordance with this aspect of the invention, detecting the presence or absence of a phosphorylated tyrosine residue in the SQEY motif can be carried out using any of the aforementioned techniques and antibodies. In addition, phosphorylation can also be detected indirectly by measuring the depletion of ATP from the reaction as described by Munagala et al., “Identification of Small Molecule Ceramide Kinase Inhibitors Using a Homogeneous Chemiluminescence High Throughput Assay,” Assay Drug Dev Tech 5:65-73 (2007), which is hereby incorporated by reference in its entirety. Alternatively, the incorporation of labeled ATP (i.e., ³²P-ATP) into the protein substrate, as described herein, can also be measured.

In a preferred embodiment, the sample used in the method of screening candidate compounds useful for modulating WSTF kinase activity contains an isolated WSTF polypeptide of SEQ ID NO:6 or SEQ ID NO:7. In addition, the sample preferably contains an isolated H2A.X protein having the amino acid sequence of SEQ ID NO:1, and phosphorylation of the tyrosine residue in the SQEY motif at amino acid position 142 of SEQ ID NO:1 is detected.

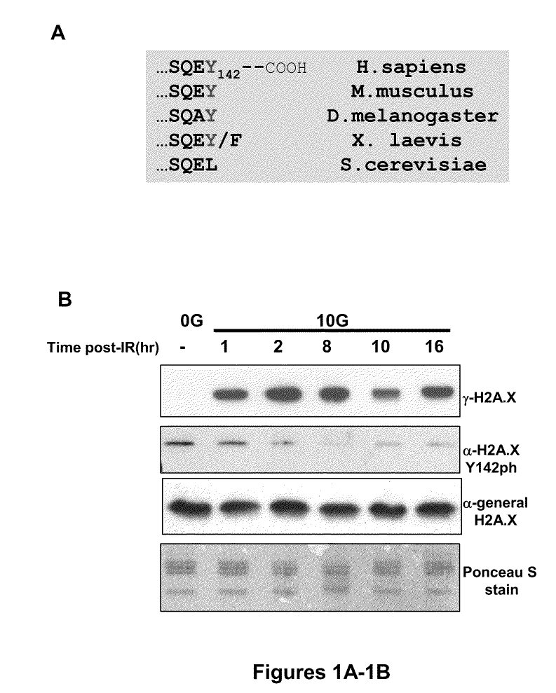

The above method of screening candidate compounds useful for modulating WSTF activity can be carried out using a whole cell sample. Any eukaryotic or prokaryotic cell sample expressing a SQEY motif sequence is suitable for use, including mammalian, insect (e.g., D. melangaster), amphibian (e.g., x. laevis), fungi (e.g., yeast), or bacteria cells. In a preferred embodiment, the cell sample comprises one or more yeast cells containing a genetically modified H2A.X gene. As described herein, a genetically modified S. cerevisiae strain containing a leucine to tyrosine mutation in its H2A.X SQEL motif (FIG. 1A; SEQ ID NO: 20) is an exemplary cell sample for use in this screening method.

Detecting the absence of a phosphorylated tyrosine residue in the SQEY motif identifies a compound useful for inhibiting WSTF kinase activity. To identify candidate compounds that mimic WSTF kinase activity, the WSTF protein or polypeptide having kinase activity is excluded from the sample. Alternatively, a cell sample which does not express endogenous WSTF or a cell sample in which endogenous WSTF expression is knocked-down using an siRNA or other biomolecular approach is used. When using a sample void of WSTF kinase activity, detecting the presence of a phosphorylated tyrosine residue in the SQEY motif identifies a compound that mimics WSTF kinase activity.

The present invention is also directed to a method of screening candidate compounds useful for modulating cellular DNA damage repair. This method involves providing a candidate compound and a cell sample and contacting the candidate compound with the cell sample. The presence or absence of a phosphorylated tyrosine residue in a SQEY tyrosine phosphorylation motif (SEQ ID NO:2) in the cell sample is detected, and candidate compounds useful for modulating cellular DNA damage repair are identified based on the presence or absence of tyrosine phosphorylation.

Methods for detecting the presence or absence of a phosphorylated tyrosine residue in a SQEY motif are described supra. In a preferred embodiment, phosphorylation of the tyrosine residue in the SQEY motif at amino acid position 142 of SEQ ID NO:1 is detected.

In accordance with this aspect of the present invention, detecting the absence of a phosphotyrosine residue in the SQEY motif identifies a candidate compound that induces DNA damage repair. Alternatively, a compound that inhibits DNA damage repair is identified by contacting the candidate compound and cell sample in the presence of a known DNA damaging agent. Any known DNA damaging agent is suitable for use in this method (CASARETT & DOULL'S TOXICOLOGY THE BASIC SCIENCE OF POISONS (Curtis D. Klaassen ed., 1996) which is hereby incorporated by reference in its entirety). Following exposure of the cell sample to a known DNA damaging agent, the detection of a phosphorylated tyrosine residue in the SQEY motif identifies a candidate compound that inhibits the cellular DNA damage repair pathway.

As described supra, any number of cell samples can be utilized in this method (mammalian, insect, yeast, etc.). In a preferred embodiment the genetically modified S. cerevisiae H2A L132Y mutant strain is utilized.

Another aspect of the present invention relates to a method of screening candidate compounds useful for treating cancer. This method involves providing a candidate compound and a cell sample and contacting the candidate compound with the cell sample. The presence or absence of a phosphorylated tyrosine residue in an SQEY tyrosine phosphorylation motif (SEQ ID NO:2) in the sample is detected and candidate compounds useful for treating cancer are identified based on the presence or absence of tyrosine phosphorylation.

Methods for detecting the presence of absence of a phosphorylated tyrosine residue in an SQEY motif are described supra. In a preferred embodiment, phosphorylation of the tyrosine residue in the SQEY motif at amino acid position 142 of SEQ ID NO:1 is detected.

Preferably, the cell sample is a cancer cell sample. The cancer cell sample can be derived directly from a cancerous tissue (i.e., primary cancer cell) or can be one of the many established cancer cell lines that are well known in the art. Preferably, the cancer cell sample, is a human cancer cell sample, however, any mammalian cancer cell is suitable.

Detecting a phosphorylated tyrosine residue in the SQEY motif in the presence of a candidate compound, but not in its absence identifies a compound that is suitable for treating cancer. Such identified compounds are particularly suitable as adjunct therapies to conventional radiotherapy and chemotherapeutic approaches to cancer as described in more detail below.

In an alternative embodiment, detecting a dephosphorylated tyrosine residue in the SQEY motif in the presence of a candidate compound but not in its absence also identifies compounds that are suitable for treating cancer. Identified compounds are suitable for administration following radiotherapy and chemotherapeutic treatments to promote DNA repair in non-cancerous cells exposed to such therapy. In addition, identified compounds are appropriate for subjects having cancer who are not receiving radiotherapy or chemotherapeutic treatments.

The present invention is also directed to a method of modulating cellular DNA damage repair. This method involves administering to a cell an agent which modulates tyrosine phosphorylation of a SQEY tyrosine phosphorylation motif (SEQ ID NO:2) under conditions effective to modulate cellular DNA damage repair.

In a preferred embodiment of this aspect of the present invention, the agent modulates tyrosine phosphorylation of the tyrosine residue in the SQEY motif at amino acid position 142 of SEQ ID NO:1 in the cell. An agent that reduces tyrosine phosphorylation of the SQEY is an agent that enhances DNA damage repair. Agents useful for enhancing DNA damage repair include WSTF kinase domain inhibitors and recombinant phosphatase proteins or peptides.

WSTF kinase domain inhibitors that are suitable for carrying out the present invention include nucleic acid, protein or peptide, or small molecule inhibitors. Nucleic acid inhibitors of the WSTF kinase domain include antisense RNA molecules, short hairpin RNA molecules (shRNA), or small interfering RNA (siRNA) molecules.

siRNA are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the WSTF mRNA sequence. siRNA molecules are typically designed to a region of the mRNA target approximately 50-100 nucleotides downstream from the start codon. Upon introduction into a cell, the siRNA complex triggers the endogenous RNA interference (RNAi) pathway, resulting in the cleavage and degradation of the target mRNA molecule. Various improvements of siRNA compositions, such as the incorporation of modified nucleosides or motifs into one or both strands of the siRNA molecule to enhance stability, specificity, and efficacy have been described and are suitable for use in accordance with this aspect of the invention (see e.g., WO2004015107 to Giese et al., WO2003070918 to McSwiggen et al., WO199839352 to Imanishi et al., U.S. Patent Application Publication No. 2002/0068708 to Jesper et al.; U.S. Patent Application Publication No. 2002/0147332 to Kaneko et al; and U.S. Patent Application Publication No. 2008/0119427 to Bhat et al., which are all hereby incorporated by reference in their entirety).

Short or small hairpin RNA molecules are longer RNA sequences that make a tight hairpin turn. Like siRNA, they silence gene expression via the cellular RNA interference pathway. shRNA is cleaved by cellular machinery into siRNA. A suitable shRNA molecule that can be used in accordance with the methods of the present invention is provided as SEQ ID NO: 21 below.

| | |
|---|---|
| tgctgttgac agtgagcgcg gagatacttc gatactttat tagtgaagcc acagatgtaa | 60 |
| taaagtatcg aagtatctcc ttgcctactg cctcgga | 97 |

Alternatively, the WSTF kinase domain inhibitor can be a peptide or protein inhibitor, such as an antibody. In a preferred embodiment, the protein inhibitor of the WSTF kinase domain is an intrabody, which selectively recognizes and binds to the intracellular WSTF kinase domain, comprising amino acids 1-391 of SEQ ID NO:3. A preferred intrabody for use in accordance with the present invention would selectively recognize and bind to an epitope of the WSTF kinase domain comprising amino acids 330-345 of SEQ ID NO:3.

Intrabodies are generally obtained by selecting a single variable domain from variable regions of an antibody having two variable domains (i.e., a heterodimer of a heavy chain variable domain and a light chain variable domain). Methods for obtaining heavy chain-light chain heterodimers are described by Kohler et al., “Continuous Cultures of Fused Cells Secreting Antibody of Predefined Specificity,” Nature 256:495-7 (1975); Campbell et al., Monoclonal Antibody Technology The Production and Characterization of Rodent and Human Hybridomas, in, LABORATORY TECHNIQUES IN BIOCHEMISTRY AND MOLECULAR BIOLOGY (Burdon et al., eds., 1985), which are hereby incorporated by reference in their entirety. Single chain Fv fragments which are the minimal recombinant antigen-binding fragments of antibodies are the most commonly used format for intrabody development. However, other suitable formats include Fab fragments, ScFv-Ck fusion proteins, single chain diabodies, V_{H}-C_{H}1 fragments, and even whole IgG molecules (see Kontermann R. E., “Intrabodies as Therapeutic Agents,” Methods 34:163-70 (2004), which is here by incorporated by reference in its entirety).

Intrabodies can be bi- or multi-functional. In addition to the antigen binding region, intrabodies can comprise other polypeptide regions defining a bioactive or function domain. Suitable functional domains include an enzyme, e.g., a protease, which can lead to the proteolysis of the WSTF protein. In another embodiment, the intrabody comprises a targeting signal, e.g., ubiquitin, which can target the WSTF protein to a proteasome for subsequent destruction. In yet another embodiment, the intrabody comprises a targeting signal that is capable of retargeting the intrabody bound WSTF protein to another cellular locale. Such a locale may be cytoplasmic, nuclear, lysosomal, plasma membrane-associated, endoplasmic reticulum-associated, peroxisomal, or proteasomal. In addition, the intrabodies or binding molecules of the invention may encompass any art recognized targeting signal for altering the cellular location of a heterologous polypeptide.

The intrabodies can be obtained from phage display, yeast surface display, or ribosome surface display. Methods for producing libraries of intrabodies and isolating intrabodies of interest are further described in U.S. Published Patent Application No. 20030104402 to Zauderer and U.S. Published Patent Application No. 20050276800 to Rabbitts, which are hereby incorporated by reference in their entirety. Methods for improving the stability and affinity binding characteristics of intrabodies are described in WO/2008/070363 to Zhenping and Contreras-Martinez et al., “Intracellular Ribosome Display via SecM Translation Arrest as a Selection for Antibodies with Enhanced Cytosolic Stability,” J. Mol. Biol. 372(2):513-24 (2007), which are hereby incorporated by reference in their entirety.

Alternatively, the WSTF kinase domain inhibitor can be a small molecule. ATP analogs, such as ATP-γ-S, ATP-γ-N, AP.PNP, or any of those described in U.S. Pat. No. 5,955,447 to Ingall et al. and Bagshaw C., “ATP Analogues at a Glance,” J Cell Science 114:459-460 (2001) which are hereby incorporated by reference in their entirety, are suitable for use in accordance with this aspect of the invention.

In an alternative embodiment of this aspect of the present invention, the agent that reduces tyrosine phosphorylation of the SQEY motif is a recombinant phosphatase protein, an active phosphatase polypeptide fragment thereof, or a nucleic acid molecule encoding the recombinant phosphatase protein or active phosphatase polypeptide fragment thereof. An exemplary recombinant phosphatase protein for reducing tyrosine phosphorylation of an SQEY motif is any phosphatase in the family of Eyes Absent (EYA) protein phosphatases, including mammalian EYA-1, EYA-2, EYA-3, and EYA-4, and Drosophila EYA proteins. In a preferred embodiment of the present invention, tyrosine phosphorylation of the SQEY motif of SEQ ID NO:1 (H2A.X) is reduced using recombinant EYA-2 or EYA-3 phosphatase proteins as described by Krishnan et al., “Dephosphorylation of the C-terminal Tyrosyl Residue of the DNA Damage-Related Histone H2A.X is Mediated by the Protein Phosphatase Eyes Absent,” J Biol Chem 284(24):16066-16070), which is hereby incorporated by reference in its entirety. The nucleic acid and amino acid sequences of human EYA protein phosphatases are well known in the art (see NCBI Genbank Accession Nos. NG 011735 for EYA1, NG 011673 for EYA-2, and NG 011596 for EYA-4, and NCBI Reference Sequence Nos. NM 001990 and NP 001981 for EYA-3, which are hereby incorporated by reference in their entirety). Methods of making recombinant proteins or peptides are described supra.

A related aspect of the invention involves a method of treating a subject having cancer. This method involves administering to the subject having cancer, an agent that modulates tyrosine phosphorylation of an SQEY tyrosine phosphorylation motif (SEQ ID NO:2) under conditions effective to treat the subject having cancer. In a preferred embodiment, the agent modulates tyrosine phosphorylation of the tyrosine residue in the SQEY motif at amino acid position 142 of SEQ ID NO:1.

In accordance with this aspect of the invention, administration of agents that modulate tyrosine phosphorylation of the SQEY motif are selected based on the type and stage of cancer the subject has, and other forms of therapy available to the subject. In one embodiment of this aspect of the invention, it may be desirable to administer agents which reduce tyrosine phosphorylation of the SQEY motif, thereby promoting cellular DNA damage repair mechanisms. Such treatments are suitable following radiation or chemotherapeutic treatments to facilitate DNA repair in non-cancerous cells exposed to such therapy. In addition, agents which promote DNA damage repair are suitable for subjects having cancer who are not receiving radiation or chemotherapy treatment. Agents of the present invention which promote DNA damage repair include WSTF kinase domain inhibitors and recombinant phosphatase proteins or polypeptides that dephosphorylate tyrosine residues of a SQEY motif. Accordingly, any of the WSTF kinase domain inhibitors described supra, including WSTF siRNA molecules, intrabodies, or other peptide-based binding molecules or small molecules, are suitable for administration to a subject having cancer. Likewise, a recombinant EYA phosphatase protein or polypeptide, particularly a recombinant EYA-2 or EYA-3 phosphatase protein or polypeptide, as described supra are also suitable for administration to a subject having cancer. Pharmaceutical compositions containing WSTF inhibitors or recombinant phosphatase proteins and modes of delivery and administration of such pharmaceutical compositions for the treatment of cancer are described infra.

In another embodiment of this aspect of the invention, it may be desirable to suppress DNA damage repair. Treatments for a wide variety of human solid malignancies and leukemia include the administration of ionizing radiation and chemotherapy. These treatments induce DNA double-strand breaks with the goal of causing cell, in particular, tumor cell lethality. In this context, it is desirable to suppress, rather than enhance, DNA repair mechanisms to avoid therapeutic resistance to such treatments. Agents that impair or suppress DNA damage repair will enhance the efficacy of radiation and chemotherapeutic treatments. Therefore, any agent that induces tyrosine phosphorylation of the SQEY motif is suitable for suppressing DNA damage repair and can be administered to a subject having cancer in combination with the above treatments. One exemplary agent useful for suppressing DNA damage repair is any agent that mimics WSTF kinase domain activity or induces endogenous WSTF kinase domain activity. Other agents suitable for suppressing DNA damage repair are EYA phosphatase inhibitors.

Agents mimicking WSTF kinase domain activity include recombinant proteins or peptides and the nucleic acids encoding such recombinant proteins or peptides. A WSTF kinase domain peptide having an amino acid sequence of SEQ ID NO:6 or SEQ ID NO:7 can be synthesized chemically or produced recombinantly in a host cell using the isolated nucleic acid molecule of SEQ ID NO:5 as described supra, in a suitable expression vector.

Agents that inhibit EYA phosphatase proteins, e.g., mammalian EYA-1, EYA-2, EYA-3, or EYA-4, include inhibitory nucleic acid molecules, peptides, and small molecules. Any EYA phosphatase inhibitors known in the art are suitable for use in the methods of the present invention. Exemplary EYA siRNA inhibitor molecules that are useful for inhibiting EYA mediated dephosphorylation of SQEY tyrosine phosphorylation are known in the art (see Krishnan et al., “Dephosphorylation of the C-terminal Tyrosyl Residue of the DNA Damage-Related Histone H2A.X is Mediated by the Protein Phosphatase Eyes Absent,” J Biol Chem 284(24):16066-16070 and Cook et al., “Tyrosine Dephosphorylation of H2AX Modulates Apoptosis and Survival Decisions,” Nature 458(2):591-596 (2009), which are hereby incorporated by reference in their entirety). These siRNA molecules are suitable agents for inhibiting DNA damage repair and promoting apoptosis of cancerous cells in a subject.

Methods of delivering a suitable agent to a subject having cancer, whether it be a WSTF kinase inhibitor, EYA phosphatase, an agent that induces WSTF kinase activity, or an EYA phosphatase inhibitor, will vary depending on the type of agent.

Intrabodies directed to WSTF or recombinant WSTF polypeptides can be administered via gene therapy. The general approach involves the introduction of a nucleic acid molecule encoding an intrabody or a nucleic acid encoding the WSTF kinase domain (SEQ ID NO:5) into cells such that one or more gene products encoded by the introduced genetic material are produced in the cells to inhibit or mimic WSTF kinase activity.

Methods of introducing nucleic acid molecule encoding the polypeptide or intrabody of interest into vectors suitable for gene therapy delivery are described supra. Gene therapy vectors can be delivered to a subject by, for example, intravenous injection, local administration (see U.S. Pat. No. 5,328,470 to Nabel et al., which is hereby incorporated by reference in its entirety) or by stereotactic injection (see e.g., Chen et al. “Gene Therapy for Brain Tumors: Regression of Experimental Gliomas by Adenovirus Mediated Gene Transfer In Vivo,” PNAS 91:3054-3057 (1994), which is hereby incorporated by reference in its entirety). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. Gene therapy vectors typically utilize constitutive regulatory elements which are responsive to endogenous transcriptions factors. In a preferred embodiment, the gene therapy vectors encoding the intrabody or polypeptide is an expression vector derived from a virus that is an adenovirus, adeno-associated virus, retrovirus, lentivirus, or herpes virus. Adenoviral viral vector gene delivery vehicles can be readily prepared and utilized given the disclosure provided in Berkner, “Development of Adenovirus Vectors for the Expression of Heterologous Genes,” Biotechniques 6:616-627 (1988) and Rosenfeld et al., “Adenovirus-Mediated Transfer of a Recombinant Alpha 1-Antitrypsin Gene to the Lung Epithelium In Vivo,” Science 252:431-434 (1991), WO 93/07283 to Curiel et al., WO 93/06223 to Perricaudet et al., and WO 93/07282 to Curiel et al., which are hereby incorporated by reference in their entirety. Adeno-associated viral gene delivery vehicles can be constructed and used to deliver a gene to cells as described in Chatterjee et al., “Dual-Target Inhibition of HIV-1 In Vitro by Means of an Adeno-Associated Virus Antisense Vector,” Science 258:1485-1488 (1992); Walsh et al., “Regulated High Level Expression of a Human Gamma-Globin Gene Introduced Into Erythroid Cells by an Adeno-Associated Virus Vector,” Proc. Nat'l. Acad. Sci. 89:7257-7261 (1992); Walsh et al., “Phenotypic Correction of Fanconi Anemia in Human Hematopoietic Cells With a Recombinant Adeno-Associated Virus Vector,” J. Clin Invest. 94:1440-1448 (1994); Flotte et al., “Expression of the Cystic Fibrosis Transmembrane Conductance Regulator From a Novel Adeno-Associated Virus Promoter,” J. Biol. Chem. 268:3781-3790 (1993); Ponnazhagan et al., “Suppression of Human Alpha-Globin Gene Expression Mediated by the Recombinant Adeno-Associated Virus 2-Based Antisense Vectors,” J. Exp. Med. 179:733-738 (1994); and Zhou et al., “Adeno-associated Virus 2-Mediated Transduction and Erythroid Cell-Specific Expression of a Human Beta-Globin Gene,” Gene Ther. 3:223-229 (1996), which are hereby incorporated by reference in their entirety. In vivo use of these vehicles is described in Flotte et al., “Stable in Vivo Expression of the Cystic Fibrosis Transmembrane Conductance Regulator With an Adeno-Associated Virus Vector,” Proc. Nat'l Acad. Sci. 90:10613-10617 (1993); and Kaplitt et al., “Long-Term Gene Expression and Phenotypic Correction Using Adeno-Associated Virus Vectors in the Mammalian Brain,” Nature Genet. 8:148-153 (1994), which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; and U.S. Pat. No. 5,885,808 to Spooner et al.; and U.S. Pat. No. 5,871,727 to Curiel, which are hereby incorporated by reference in their entirety.

Retroviral vectors which have been modified to form infective transformation systems can also be used to deliver nucleic acid encoding a desired protein or polypeptide into a target cell. One such type of retroviral vector is disclosed in U.S. Pat. No. 5,849,586 to Kriegler et al., which is hereby incorporated by reference.

Regardless of the type of infective transformation system employed, it should be targeted for delivery of the nucleic acid to a specific cell type. For example, for delivery of the nucleic acid into tumor cells, a high titer of the infective transformation system can be injected directly within the tumor site so as to enhance the likelihood of tumor cell infection. The infected cells will then express the desired protein product, for example a WSTF kinase domain, to immolate the cancer cell.

Non-viral gene delivery vehicles are also a means to effect cell-specific delivery of the therapeutic plasmids for the present invention. These are traditionally antibodies or single-chain Fv antibodies that are coupled or fused to DNA complexing agents (see Uherek et al., “Long-Term Gene Expression and Phenotypic Correction Using Adeno-Associated Virus Vectors in the Mammalian Brain,” J. Biol. Chem. 273:8835-8841 (1998); Foster et al., “HER2-Targeted Gene Transfer,” Human Gene Ther. 8:719-727 (1997); Chen et al., “Design of a Genetic Immunotoxin to Eliminate Toxin Immunogenicity,” Gene Ther. 2:116-123 (1995), which are hereby incorporated by reference in their entirety). This class of gene delivery vehicles also includes antibodies or their fragments coupled to liposomes (U.S. Pat. Nos. 4,925,661, 4,957,735, and 6,008,202 to Huang, which are hereby incorporated by reference in their entirety).

Another approach for delivering recombinant proteins or peptides as well as siRNA molecules of the present invention directly into cells involves the use of liposomes. Liposomes are vesicles comprised of one or more concentrically ordered lipid bilayers which encapsulate an aqueous phase. They are normally not leaky, but become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug, in this case recombinant WSTF kinase polypeptide or siRNA molecule, at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be regulated.

In contrast to passive drug release, active drug release involves using an agent to induce a permeability change in the liposome vesicle. Liposome membranes can be constructed so that they become destabilized when the environment becomes acidic near the liposome membrane (see e.g., Wang et al., “pH-Sensitive Immunoliposomes Mediate Target-Cell-Specific Delivery and controlled Expression of a Foreign Gene in Mouse,” Proc. Natl. Acad. Sci. USA 84:7851 (1987), which is hereby incorporated by reference). When liposomes are endocytosed by a target cell, for example, they can be routed to acidic endosomes which will destabilize the liposome and result in drug release. Alternatively, the liposome membrane can be chemically modified such that an enzyme is placed as a coating on the membrane which slowly destabilizes the liposome. A liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., “Diffusion of Univalent Ions Across the Lamellae of Swollen Phospholipids,” J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., which are hereby incorporated by reference in their entirety.

An alternative approach for delivery of proteins or polypeptides involves the conjugation of the desired protein or polypeptide to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of proteins or polypeptides involves preparation of chimeric proteins according to U.S. Pat. No. 5,817,789 to Heartlein et al., which is hereby incorporated by reference in its entirety. A chimeric protein suitable for use in the methods of the present invention contains a ligand binding domain and the kinase domain of WSTF (SEQ ID NO:6 or SEQ ID NO:7) or a fragment of variant thereof. The ligand binding domain is specific for cell surface receptors located on a target cell. Thus, when the chimeric protein is delivered intravenously or otherwise introduced into blood or lymph, the chimeric protein will be selectively taken up and internalized by the target cell.

In practicing the method of the present invention, agents suitable for treating a subject having cancer can be administered using any method standard in the art. The agents, in their appropriate delivery form, can be administered orally, intradermally, intramuscularly, intraperitoneally, intravenously, subcutaneously, or intranasally. The compositions of the present invention may be administered alone or with suitable pharmaceutical carriers, and can be in solid or liquid form, such as tablets, capsules, powders, solutions, suspensions, or emulsions.

The agents of the present invention may be orally administered, for example, with an inert diluent, or with an assimilable edible carrier, or it may be enclosed in hard or soft shell capsules, or it may be compressed into tablets, or they may be incorporated directly with the food of the diet. Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present invention in such therapeutically useful compositions is such that a suitable dosage will be obtained.

Also specifically contemplated are oral dosage forms of the agents of the present invention. The agents may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (A. Abuchowski, Soluble Polymer-Enzyme Adducts, in ENZYMES AS DRUGS 367-383 (Holcenberg et al., eds., 1981), which are hereby incorporated by reference in their entirety). Other polymers that could be used are poly-1,3-dioxolane and poly-1,3,6-tioxocane. Preferred for pharmaceutical usage, as indicated above, are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, sucrulose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The agents of the present invention may also be administered parenterally. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt

The agents of the present invention may also be administered directly to the airways in the form of an aerosol. This form of administration is particularly suited for siRNA delivery. For use as aerosols, the agent of the present invention in solution or suspension may be packaged in a pressurized aerosol container together with suitable propellants, for example, hydrocarbon propellants like propane, butane, or isobutane with conventional adjuvants. The agent of the present invention also may be administered in a non-pressurized form such as in a nebulizer or atomizer.

Effective doses of the compositions of the present invention, for the treatment of cancer vary depending upon many different factors, including type and stage of cancer, means of administration, target site, physiological state of the patient, other medications or therapies administered, and physical state of the patient relative to other medical complications. Treatment dosages need to be titrated to optimize safety and efficacy.

### EXAMPLES

The Examples set forth below are for illustrative purposes only and are not intended to limit, in any way, the scope of the present invention.

### Example 1

### Plasmids and Cell Culture

Tyr142 mutant constructs were derived from WT human H2A.X plasmids (Open Biosystems) using Quickchange kit (Stratagene) and verified by DNA sequencing.

Primers:
Y142L forward:

(SEQ ID NO: 22)

5′-caagaaggccacccaggcctcccaggagctctaag-3′;
Y142L Reverse:

(SEQ ID NO: 23)

5′-cttagagctcctgggaggcctgggtggccttcttg-3′;
Y142F forward:

(SEQ ID NO: 24)

5′-caagaaggccacccaggcctcccaggagttctaag-3′;
Y142L Reverse:

(SEQ ID NO: 25)

5′-cttagaactcctgggaggcctgggtggccttcttg-3′.

These plasmids were amplified by PCR and cloned into pTOPO vectors (Invitrogen).

Primers:
Forward:

(SEQ ID NO: 26)

5′-cacctcgggccgcggcaagactg-3′
Reverse: WT:

(SEQ ID NO: 27)

5′-cttagtactcctgggag-3′;
Y142L:

(SEQ ID NO: 28)

5′-cttagagctcctgggag-3′;
Y142F:

(SEQ ID NO: 29)

5′-cttagaactcctgggag-3′.

For generating N-terminal Flag tagged constructs, the WT or mutant H2A.X coding region in the pTOPO vectors were cloned in-frame between the BamH1 and Xho1 sites of the pCMV-Tag2A vectors (Stratagene). Tagged or untagged constructs were cloned into MSCV-puro vectors (gifts from Dr. Herr's lab, University of Zurich, Switzerland). To generate constructs for recombinant protein expression in E. Coli, the coding regions of the WT or Y142F H2A.X of the untagged vectors (above) were cloned into pET28 vectors (Novagen), in-frame with the N-terminal 6×His tag.

MSCV virus production and infection followed standard protocols (Zhou et al., The DNA Damage Response: Putting Checkpoints in Perspective,” Nature 408:433-439 (2000), which is hereby incorporated by reference in its entirety). In brief, MSCV viruses were packaged in Phoenix cells and used to infect H2A.X −/− MEFs (gifts from Dr. A. Nussenzweig's Lab, NIH, Bethesda, Md.), followed by puromycin selection. For each construct, three independent lines were derived. The expression levels of H2A.X were checked by immunoblot and immunofluorescence.

The full-length WSTF (human) construct was a gift from Dr. Varga-Wewasz's lab (Babraham Institute, Cambridge, UK). To generate a series of truncated WSTF constructs for insect cell expression, the PCR fragments of the corresponding regions of the WSTF gene were cloned into a modified pFASTBac vector (Invitrogen), which contains an N-terminal GST and a C-terminal His-tag sequence. The C338A mutant (1-359) was derived from the WT 1-359 construct, using the QuickChange site-directed mutagenesis kit (Stratagene) and was verified by DNA sequencing. To generate constructs for protein expression in E coli, the PCR product of the N-motif (1-205) was cloned into pGex6p vector (GE Healthcare) in-frame with the N-terminal GST tag, and the PCR product of the C-motif (208-345) was cloned into a modified pRSFDuet-1 (Novagen) vector in-frame with the N-terminal MBP tag. To generate constructs for mammalian cell expression, the PCR products (the forward primer contains a BamH1 and reverse contains a Xho1 site sequence) of the corresponding fragments of the human WSTF gene were cloned in between the BamH1 and Xho1 sites of pTAG3A vectors (Stratagene), in-frame with the N-terminal Myc epitope tag. The tagged constructs were cloned into MSCV-puro vectors.

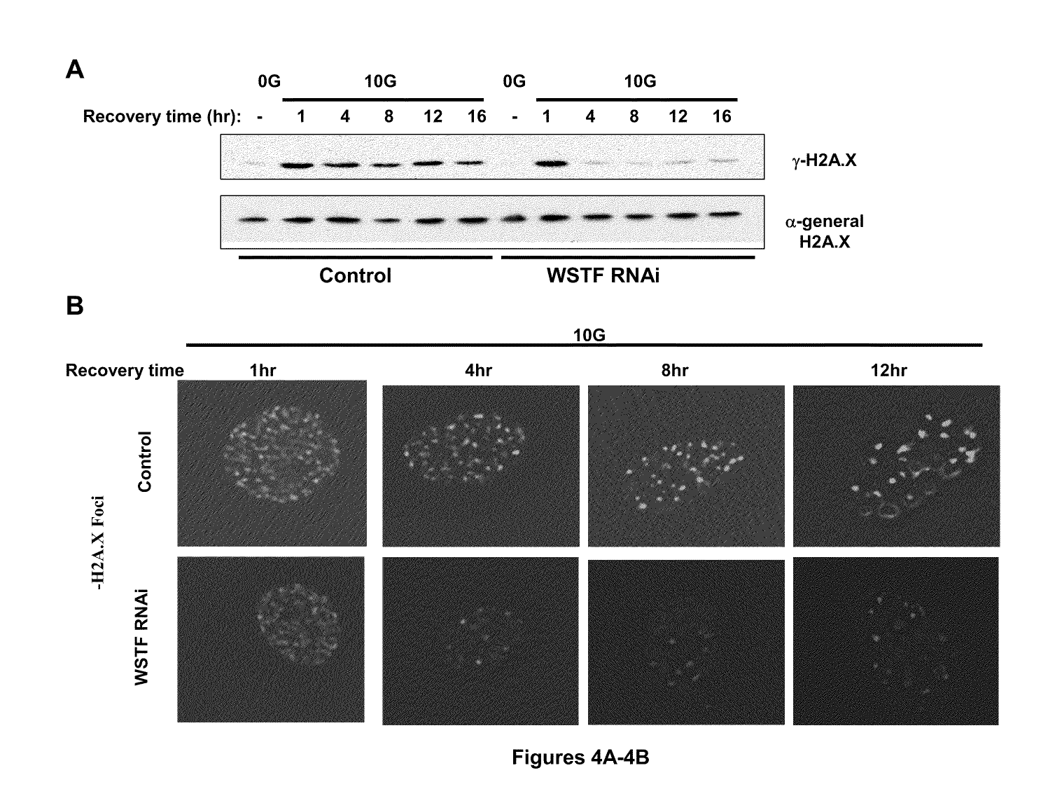

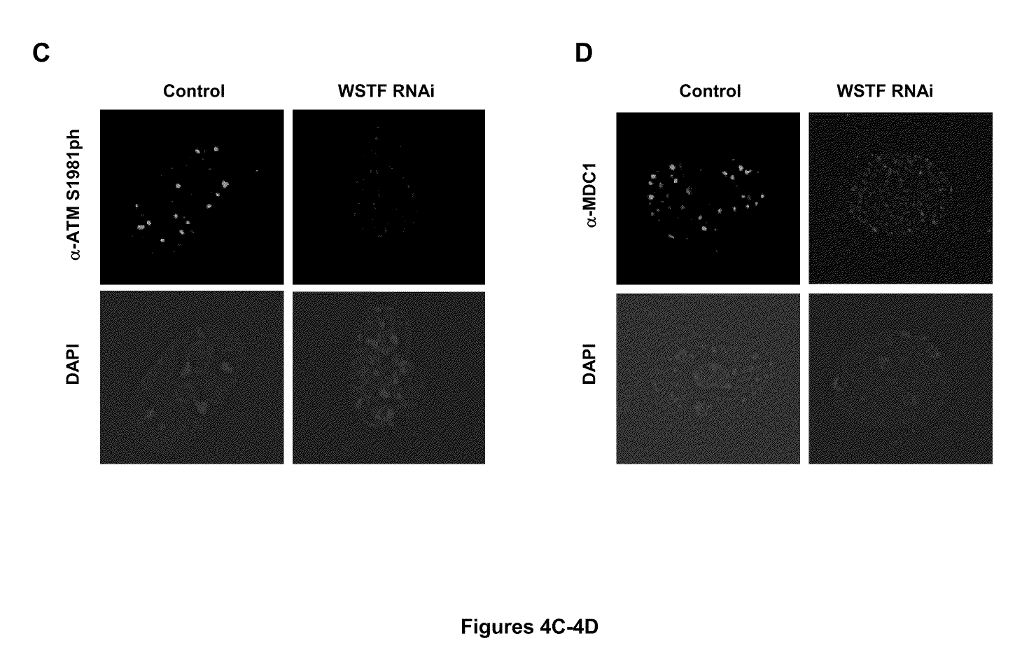

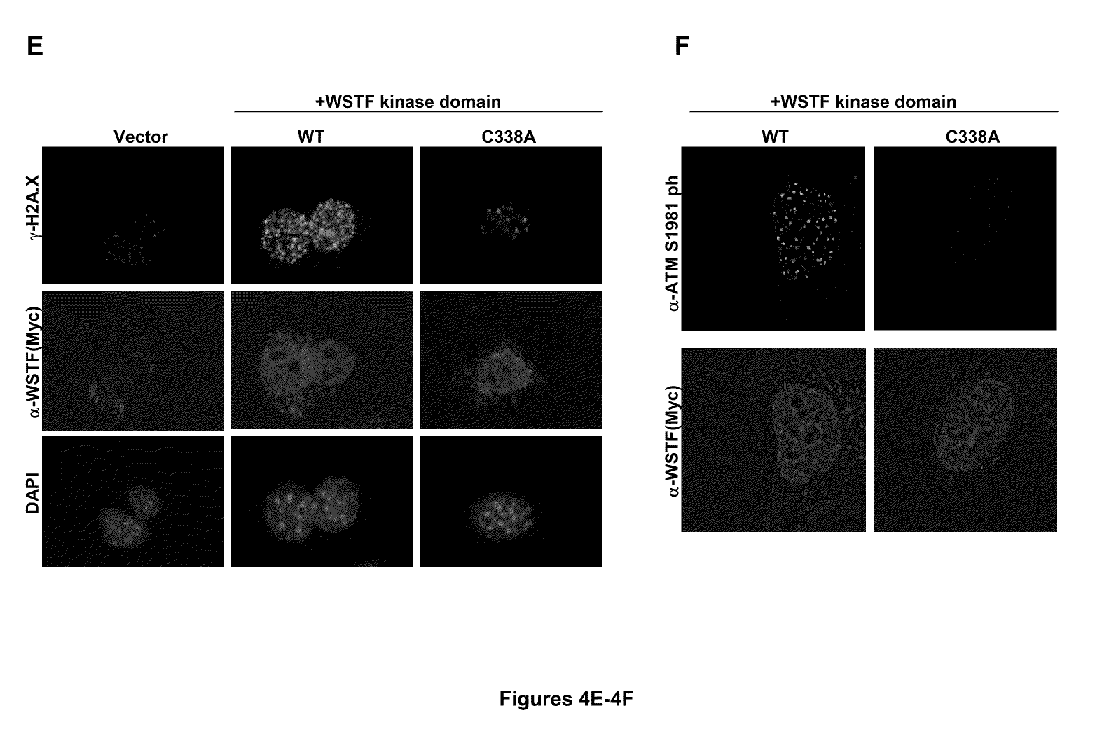

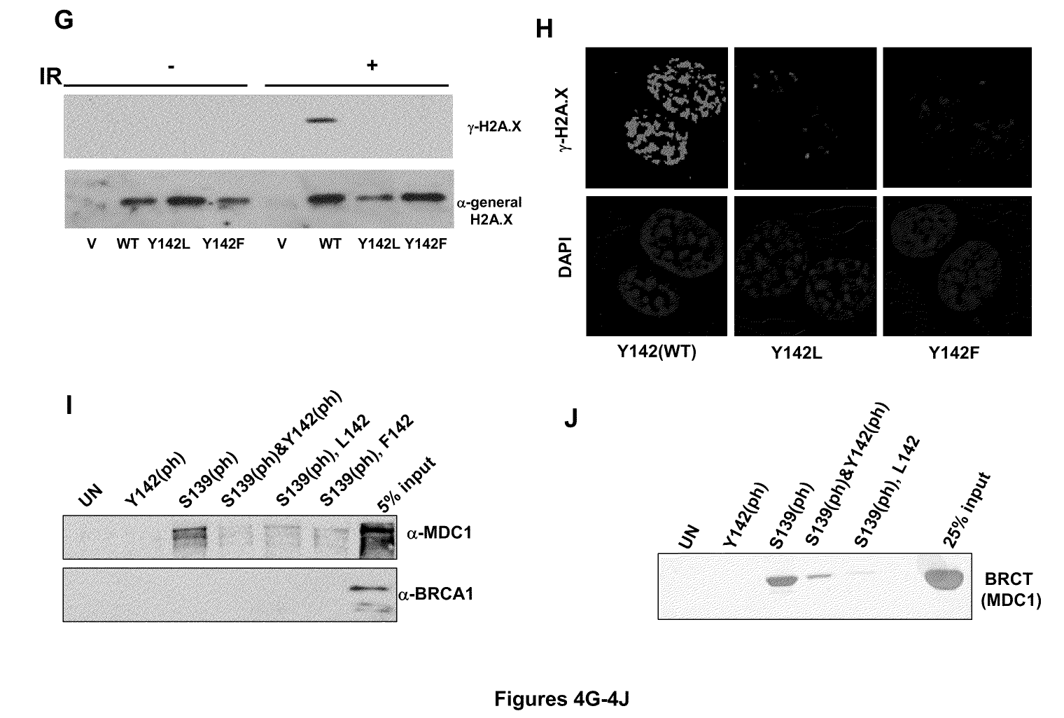

WSTF RNAi cell lines were generated using recombinant pShag-2 vectors containing shRNA constructs targeting the mouse WSTF gene (Clone ID: V2MM_{—}17087, Open Biosystems). Three independent lines were generated by infecting virus carrying these vectors into NIH3T3 cells and propagated under puromycin. In FIG. 4, the WSTF RNAi cells were infected with recombinant MSCV viruses carrying the WT (1-359) or the mutant (C338A) constructs.

### Example 2

### Purification of H2A.X Containing Mononucleosome and its Associated Protein Factors

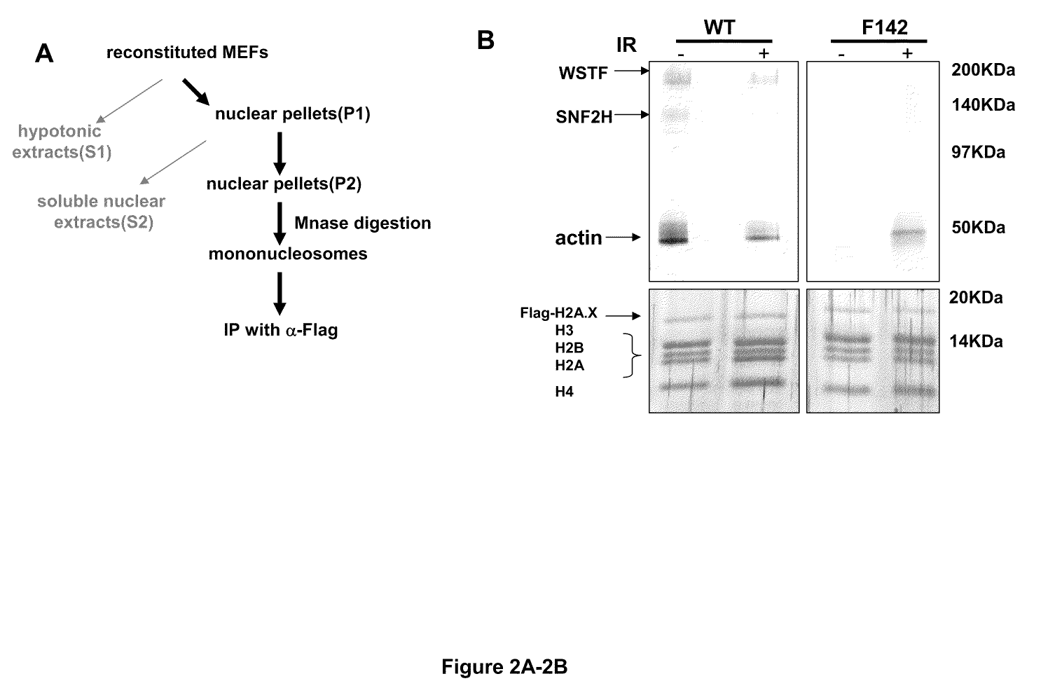

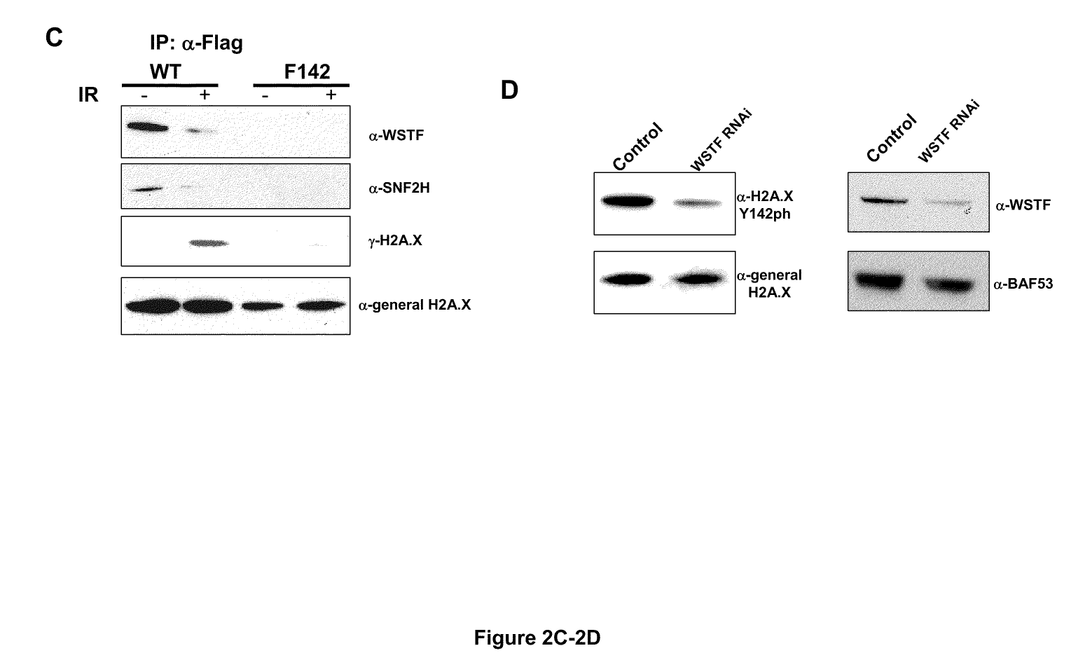

MEF fractionation and chromatin pellet isolation (FIG. 2) were performed as described (Redon et al., “Histone H2A Variants H2AX and H2AZ,” Curr Opin Genet Dev 12:162-169 (2002), which is hereby incorporated by reference in its entirety). Chromatin pellets were briefly digested with Mnase (Sigma) and the generation of mononucleosomes was monitored by electrophoresis (Celeste et al., “Genomic Instability in Mice Lacking Histone H2AX,” Science 296:922-927 (2002), which is hereby incorporated by reference in its entirety). Fifty micro-liter FLAG-M2 agarose beads were used for immunoprecipitating the total mononucleosomes isolated from 2×10⁸ cells. The immunocomplex was resolved on 4-12% gradient gels (Invitrogen) and then silver- or coomassie blue stained. The gel bands were isolated and subjected to mass spectrometry.

### Example 3

### Protein Identification by Mass Spectrometry

Gel-resolved proteins were digested with trypsin, batch purified on a reversed-phase micro-tip, and resulting peptide pools individually analyzed by matrix-assisted laser desorption/ionization reflectron time-of-flight (MALDI-reTOF) mass spectrometry (MS) (UltraFlex TOF/TOF; BRUKER; Bremen, Germany) for peptide mass fingerprinting (PMF), as described (Celeste et al., “H2AX Haploinsufficiency Modifies Genomic Stability and Tumor Susceptibility,” Cell 114:371-383 (2003), which is hereby incorporated by reference in its entirety). Selected peptide ions (m/z) were taken to search a “non-redundant” protein database (NR; 3,245,378 entries on 28 Jan. 2006; National Center for Biotechnology Information; Bethesda, Md.) utilizing the PeptideSearch algorithm (Matthias Mann, Max-Planck Institute for Biochemistry, Martinsried, Germany; an updated version of this program is currently available as ‘PepSea’ from Applied Biosystems/MDS Sciex; Foster City, Calif.). A molecular mass range up to twice the apparent molecular weight (as estimated from electrophoretic relative mobility) was covered, with a mass accuracy restriction of less than 40 ppm, and maximum one missed cleavage site allowed per peptide. To confirm PMF results, mass spectrometric sequencing of selected peptides was done by MALDI-TOF/TOF (MS/MS) analysis on the same prepared samples, using the UltraFlex instrument in ‘LIFT’ mode. Fragment ion spectra were taken to search NR using the MASCOT MS/MS Ion Search program (Reina-San-Martin et al., “H2AX is Required for Recombination Between Immunoglobulin Switch Regions but Not for Intra-Switch Region Recombination or Somatic Hypermutation,” J Exp Med 197:1767-1778 (2003), which is hereby incorporated by reference in its entirety), version 2.0.04 for Windows (Matrix Science Ltd., London, UK). Any tentative confirmation of a PMF result thus obtained was verified by comparing the computer-generated fragment ion series of the predicted tryptic peptide with the experimental MS/MS data.

### Example 4

### Immunoblotting and Immunofluorescence

Cells at 70-80% confluence were exposed to an ionizing radiation source (Cs) to introduce DNA damage (at 10 Gy). Samples were collected at indicated time points post irradiation. Histone extraction, cell fractionation, high salt nuclear extraction and western blotting followed standard protocols (Bassing et al., “Histone H2AX: A Dosage-dependent Suppressor of Oncogenic Translocations and Tumors,” Cell 114:359-370 (2003) and Unal et al., “DNA Damage Response Pathway Uses Histone Modification to Assemble a Double-Strand Break-Specific Cohesin Domain,” Mol Cell 16:991-1002 (2004), which is hereby incorporated by reference in its entirety). For immunofluorescence, cells were grown on cover slips and fixed with 3% paraformaldehyde. Subsequent indirect immunofluorescent staining was achieved following standard protocols (Unal et al., “DNA Damage Response Pathway Uses Histone Modification to Assemble a Double-Strand Break-Specific Cohesin Domain,” Mol Cell 16:991-1002 (2004), which is hereby incorporated by reference in its entirety). For WSTF staining, cells were extracted with a high salt buffer for 2 minutes at 4° C. before fixing, as described (Harper et al., “The DNA Damage Response: Ten Years After,” Mol Cell 28:739-745 (2007), which is hereby incorporated by reference in its entirety). Antibodies: γ-H2A.X, α-general H2A.X and α-BRCA1 (Millipore/Upstate); α-WSTF and α-Myc (Sigma); α-BAF53 and α-SNF2H (Rockefeller University, NY, and New York University, NY, respectively).

### Example 5

### Recombinant MDC1 Protein Expression and Peptide Pull-Down Assay

GST-MDC1-BRCT protein expression followed manufacturer's protocols (Stratagene). Peptides (unmodified “UN”):

| | |
|---|---|
| N-CPSGGKKATQASQEY-C; | (SEQ ID NO: 30) |
| S139(ph): | |
| N-CPSGGKKATQApSQEY-C; | (SEQ ID NO: 31) |
| Y142(ph): | |
| N-CPSGGKKATQASQEpY-C; | (SEQ ID NO: 32) |
| S139(ph) & Y142(ph): | |
| N-CPSGGKKATQApSQEpY-C; | (SEQ ID NO: 33) |
| Y142F: | |
| N-CPSGGKKATQASQEF-C; | (SEQ ID NO: 34) |
| Y142L: | |
| N-CPSGGKKATQASQEL-C; | (SEQ ID NO: 35) |
| S139(ph), Y142L: | |
| N-CPSGGKKATQApSQEL-C; | (SEQ ID NO: 36) |
| S139(ph), Y142F: | |
| N-CPSGGKKATQApSQEF-C | (SEQ ID NO: 37)) |

were conjugated to agarose bead with Sulfolink kit (Pierce). Peptide pull-down experiments were performed as described previously (Morrison et al., “INO80 and Gamma-H2AX Interaction Links ATP-dependent Chromatin Remodeling to DNA Damage Repair,” Cell 119:767-775 (2004), which is hereby incorporated by reference in its entirety).

### Example 6

### Circular Dichroism Measurement

Circular Dichroism (CD) experiments were performed on an Aviv 62A/DS (Aviv Associates, Lakewood, N.J.) spectropolarimeter. Spectra (260 nm to 200 nm) were collected with a cell path length of 0.1 cm and bandwidth of 1 nm. Samples were diluted to 10 uM with 20 mM Tris-HCl, pH 7.5, 100 mM KCl, 2 mM DTT. Protein concentrations were determined with their 280 nm absorbance.

### Example 7

### Automated Chemical (‘Edman’) Protein Sequencing

Protein samples were analyzed with a Procise 494 instrument from Applied Biosystems (AB) as described (Downs et al., “Binding of Chromatin-Modifying Activities to Phosphorylated Histone H2A at DNA Damage Sites,” Mol Cell 16:979-990 (2004), which is hereby incorporated by reference in its entirety). Stepwise liberated PTH-amino acids were identified using an “on-line” HPLC system (AB) equipped with a PTH C18 (2.1×220 mm; 5 micron particle size) column (AB).

### Example 8

### Preparation of Recombinant WSTF Protein from Insect Cells

Recombinant baculovirus carrying the full length WSTF (Flag-epitope-tagged at C terminus) was produced using the BaculoGold-BEVS Kit (BD Biosciences). The full length WSTF proteins were purified using a published protocol (van Attikum et al., “Recruitment of the INO80 Complex by H2A Phosphorylation Links ATP-Dependent Chromatin Remodeling With DNA Double-Strand Break Repair,” Cell 119:777-788 (2004), which is hereby incorporated by reference in its entirety). The viruses that express truncated WSTF constructs were produced with the Bac-to-Bac (Invitrogen) method, all of which were tagged with an N-terminal GST tag and a C-terminal 6×His tag. Baculoviruses were produced in Sf9 cells, and proteins were expressed in Hi5 suspension cells. For truncated WSTF protein purification, harvested Hi5 cells were suspended in 0.4 M KCl, 20 mM Tris, pH 7.5, supplemented with the EDTA-free Complete™ Protease Inhibitor Cocktail tablet (Roche). Whole cell lysates were purified through GSTrap columns (GE Healthcare). After removal of the GST tags by PreScission Protease digestion (GE Healthcare), the protein mixtures were purified by HiTrap SP (GE Healthcare). The recombinant WSTF proteins (peaks form at ˜200 mM KCL, 20 mM Tris pH 7.5) were further purified with Superdex 75 (GE Healthcare) in the elution buffer (50 mM KCl, 20 mM Tris, pH 7.5). The monomeric peaks (˜60 KDa) were collected and concentrated for further use. The C338A mutant protein was expressed and purified as the WT protein described above.

### Example 9

### Protein Preparation in E. coli

For co-expression of the N- and C-motifs of WSTF, the recombinant plasmid vectors carrying the N- and C-motif constructs (above) were transformed into E. coli host strain Rosetta2 (DE3) (Novagen) for protein expression under triple antibiotic selection (ampicillin, kanamycin, and chloramphenicol) in LB medium. After overnight induction by 0.4 mM isopropyl β-D-thiogalactoside at 25° C., cells were harvested in buffer 0.4 M KCl, 20 mM Tris, pH 7.5. Co-expressed proteins were affinity purified using Amylose resin (New England Biolabs, MA) against the MBP tag. Maltose-eluted samples were purified through a Superdex 200 column (GE Healthcare) in the elution buffer (50 mM KCL, 20 mM Tris, pH 7.5). Most proteins were eluted in a single peak that contains GST-tagged WAC N- and MBP-tagged WAC C-motif with a stoichiometry of 1:1.

The N- or C-motif were also expressed separately in Rosetta2 (DE3) (Novagen) cells in parallel. The N-motif was purified with GSTrap column and Superdex 200 (GE Healthcare) and C-motif was purified with Amylose resin (New England Biolabs, MA) and Superdex 200 (GE Healthcare). Most proteins were eluted in a monomeric peak (˜50 KDa).

### Example 10

### Histone Protein Purification and In Vitro Assembly of Histone Octamers and Mononucleosomes

Individual free histone proteins (H2A.X, H₂B, H3 and H4) were expressed and purified using a standard protocol (Kusch et al., “Acetylation by Tip60 is Required for Selective Histone Variant Exchange at DNA Lesions,” Science 306:2084-2087 (2004), which is hereby incorporated by reference in its entirety). N-terminal 6×His-epitope-tagged WT and F142H2A.X proteins were purified with Ni Sepharose6 columns (GE Healthcare). The assembly of the histone octamers followed standard protocols (Kusch et al., “Acetylation by Tip60 is Required for Selective Histone Variant Exchange at DNA Lesions,” Science 306:2084-2087 (2004), which is hereby incorporated by reference in its entirety). Four recombinant histone proteins were denatured in 8M Guanidine-HCl solutions and dialyzed to 2M NaCl. The crude histone octamers were further purified through a Superdex 75 column (GE Healthcare). Mononucleosomes were assembled as described (Park et al., “Mammalian SWI/SNF Complexes Facilitate DNA Double-Strand Break Repair by Promoting Gamma-H2AX Induction,” EMBO J. 25:3986-3997 (2006), which is hereby incorporated by reference in its entirety). The PCR products of the DNA template (nucleosome positioning sequence (clone 601) (Stewart et al., “MDC1 is a Mediator of the Mammalian DNA Damage Checkpoint,” Nature 421:961-966 (2003), which is hereby incorporated by reference in its entirety) was initially incubated in a reaction buffer (Park et al., “Mammalian SWI/SNF Complexes Facilitate DNA Double-Strand Break Repair by Promoting Gamma-H2AX Induction,” EMBO J. 25:3986-3997 (2006), which is hereby incorporated by reference in its entirety) supplemented with 2M NaCl and diluted in the same reaction buffer stepwise until the NaCl concentration reached 200 mM. The assembly of mononucleosomes was checked by electrophoresis on 5% native PAGE gels.

### Example 11

### In Vitro Kinase Assays

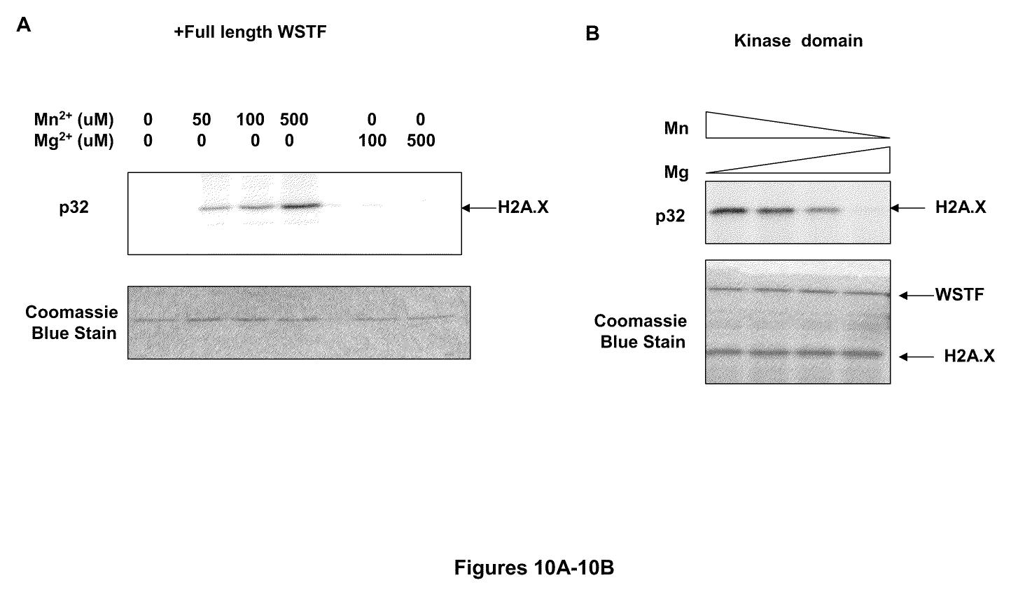

Recombinant full-length (50 ng-100 ng) or truncated WSTF proteins (0.1-0.5 ug) were incubated with free histone proteins (1-4 μg), histone octamers or nucleosomes assembled in vitro (500 ng-1 μg) in 20 mM Tris pH 7.4 and 150 mM NaCl supplemented with γ-³²P-ATP (NEN) and 1 mM MnCl₂ for 45 minutes. In FIG. 10, 0.1-1 mM of MnCl₂ or MgCl₂ was supplemented as indicated. The reaction was stopped by adding 2 mM EDTA. The reaction mixtures were separated by SDS-PAGE, stained with Coomassie blue and dried. The dried gels were exposed against phosphorimager screens (FujiFilm). The autoradiography images were scanned in phosphorimager (FujiFilm) and analyzed by Image Reader FLA-5000.

### Example 12

### Phosphorylation of H2A.X at Tyr142 is a Novel Phosphorylation Mark that is Regulated in Response to DNA Damage

A role in the DNA damage response for mammalian H2A.X (γ-H2A.X) is well documented, although its regulation and underlying mechanism of action is only partially understood (reviewed in van Attikum et al., “Recruitment of the INO80 Complex by H2A Phosphorylation Links ATP-Dependent Chromatin Remodeling With DNA Double-Strand Break Repair,” Cell 119:777-788 (2004), which is hereby incorporated by reference in its entirety). Phosphorylation of S139 in the C-terminal tail of H2A.X is responsible for the recruitment of many proteins to sites of DNA damage and directly recruits Mdc1 (Stewart et al., “MDC1 is a Mediator of the Mammalian DNA Damage Checkpoint,” Nature 421:961-966 (2003); Goldberg et al., “MDC1 is Required for the Intra-S-Phase DNA Damage Checkpoint,” Nature 421:952-956 (2003); Lou et al., “MDC1 is Coupled to Activated CHK2 in Mammalian DNA Damage Response Pathways,” Nature 421:957-961 (2003); Stucki et al., “MDC1 Directly Binds Phosphorylated Histone H2AX to Regulate Cellular Responses to DNA Double-Strand Breaks,” Cell 123:1213-1226 (2005); Lou et al., “MDC1 Maintains Genomic Stability by Participating in the Amplification of ATM-Dependent DNA Damage Signals,” Mol Cell 21:187-200 (2006); and Lee et al., “Structure of the BRCT Repeat Domain of MDC1 and its Specificity for the Free COOH-terminal End of the Gamma-H2AX Histone Tail,” J Biol Chem 280:32053-32056 (2005), which are hereby incorporated by reference in their entirety), a critical mediator of DNA damage response. Further inspection of the C-terminus of H2A.X revealed a tyrosine (Tyr142 in mammals) that exists in metazoans, such as human, rodents, and Drosophila, but is absent in unicellular eukaryotes such as yeast (FIG. 1A). Interestingly, two forms of H2A.X in the Xenopus genome, which are different at this residue (the “F” and “Y” form, FIG. 1A), are differentially expressed during development. Although recent studies suggest a role for Tyr142 in recruiting Mdc1 (Stewart et al., “MDC1 is a Mediator of the Mammalian DNA Damage Checkpoint,” Nature 421:961-966 (2003); Goldberg et al., “MDC1 is Required for the Intra-S-Phase DNA Damage Checkpoint,” Nature 421:952-956 (2003); Lou et al., “MDC1 is Coupled to Activated CHK2 in Mammalian DNA Damage Response Pathways,” Nature 421:957-961 (2003); Stucki et al., “MDC1 Directly Binds Phosphorylated Histone H2AX to Regulate Cellular Responses to DNA Double-Strand Breaks,” Cell 123:1213-1226 (2005); Lou et al., “MDC1 Maintains Genomic Stability by Participating in the Amplification of ATM-Dependent DNA Damage Signals,” Mol Cell 21:187-200 (2006); and Lee et al., “Structure of the BRCT Repeat Domain of MDC1 and its Specificity for the Free COOH-terminal End of the Gamma-H2AX Histone Tail,” J Biol Chem 280:32053-32056 (2005), which are hereby incorporated by reference in their entirety) the in vivo function of Tyr142, especially in the regulation of γ-H2A.X, remains unclear (Stucki et al., “MDC1 Directly Binds Phosphorylated Histone H2AX to Regulate Cellular Responses to DNA Double-Strand Breaks,” Cell 123:1213-1226 (2005), which is hereby incorporated by reference in its entirety).

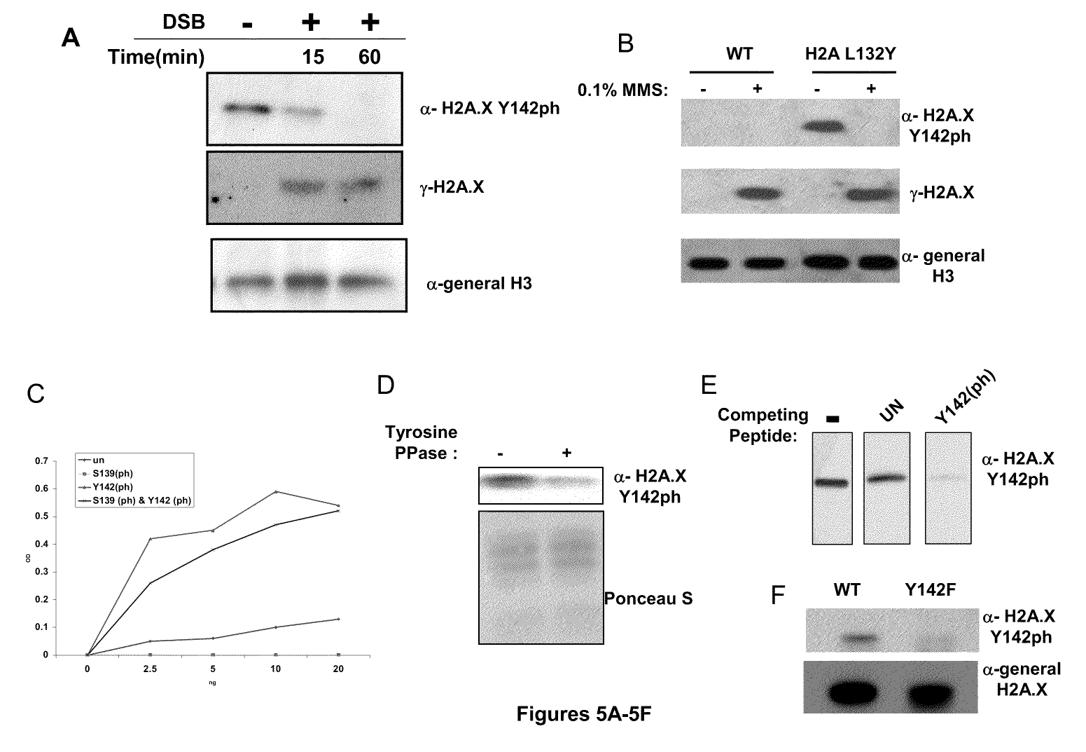

Whether Tyr142 is phosphorylated in H2A.X under certain physiological conditions was investigated (FIG. 1B). Preliminary studies using a pan anti-phosphotyrosine antibody indicated that H2A.X was phosphorylated prior to DNA damage. To investigate further whether Tyr142 is indeed phosphorylated, an antibody raised against an extreme C-terminal peptide containing phosphorylated Tyr142 of H2A.X was generated and shown to be highly selective for H2A.X-Tyr142ph (hereafter, α-H2A.X-Y142ph; See FIGS. 5C-F for characterization of antibody). In MEF cells, Tyr142 is constitutively phosphorylated under normal growth conditions and becomes gradually dephosphorylated during the DNA damage response, while γ-H2A.X increases (FIG. 1B). Whether this observation is conserved in other organisms was then investigated. It has been reported that H2A.X is highly enriched in Xenopus oocytes and eggs (Dimitrov et al., “Remodeling Sperm Chromatin in Xenopus laevis Egg Extracts: The Role of Core Histone Phosphorylation and Linker Histone B4 in Chromatin Assembly,” J Cell Biol 126:591-601 (1994) and Kleinschmidt et al., “DNA-Dependent Phosphorylation of Histone H2A.X During Nucleosome Assembly in Xenopus laevis Oocytes: Involvement of Protein Phosphorylation in Nucleosome Spacing,” EMBO J 10:3043-3050 (1991), which are hereby incorporated by reference in their entirety). Thus, whether its Tyr142 phosphorylation status would be altered in response to DNA damage treatment, in the context of Xenopus early embryonic development was examined. Similar to mammalian cells, Tyr142 phosphorylation levels are greatly decreased in response to DNA damage (FIG. 5A).

Since the major form of H2A in yeast carries the “SQEL” motif (FIG. 1A and Redon et al., “Histone H2A Variants H2AX and H2AZ,” Curr Opin Genet Dev 12:162-169 (2002), which is hereby incorporated by reference in its entirety), whether a point mutant mimicking the mammalian “SQEY” motif would constitute a new phospho-acceptor was investigated. Similar to the mammalian cells, this mutant yeast strain (L132Y) is strongly reactive with the α-H2A.X-Y142ph antibody, with reactivity greatly diminished following DNA damage treatment (FIG. 5B). Collectively, these unexpected results raise the intriguing possibility that a conserved phosphotyrosine pathway may regulate H2A.X phosphorylation in mammalian, Xenopus, and yeast cells, even though yeast H2A lacks a C-terminal tyrosine naturally. These data provide an early indication that Tyr142 phosphorylation may play a role in regulating the DNA damage response of H2A.X in a wide range of organisms by previously unknown mechanisms.

### Example 13

### The WSTF-SNF2H Chromatin Remodeling Complex Interacts with H2A.X

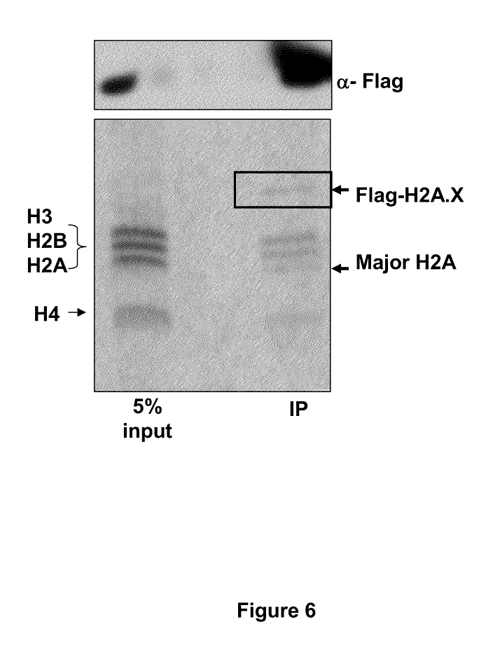

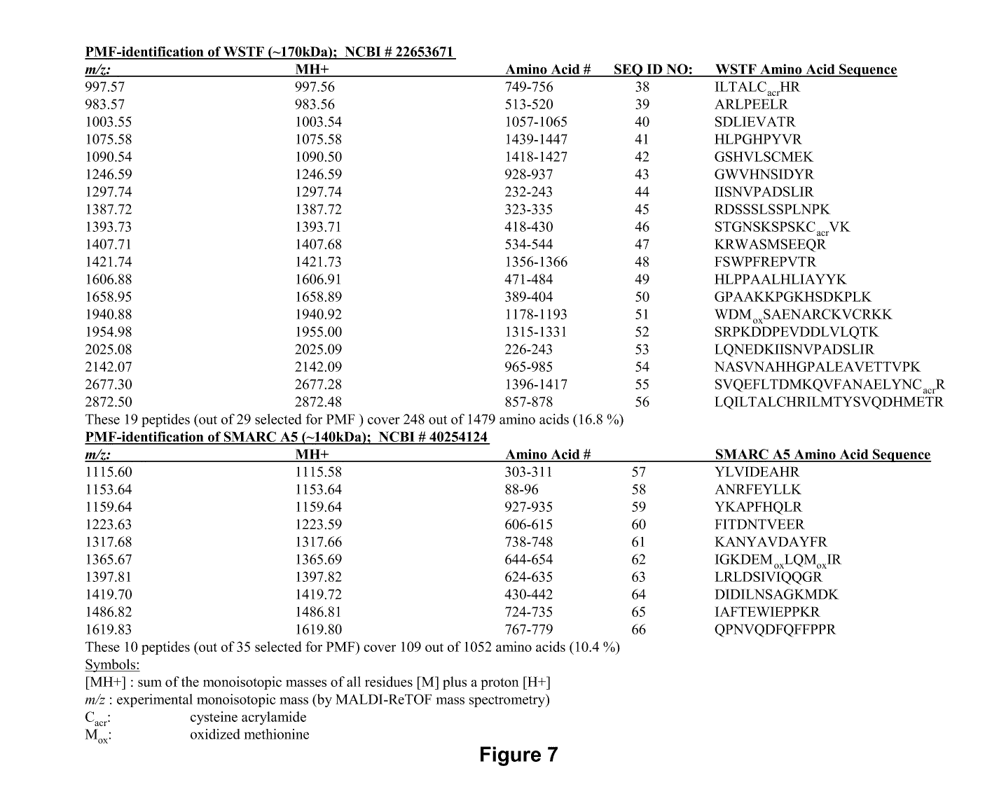

To investigate novel mechanisms that regulate H2A.X function, protein complexes directly associated with H2A.X in vivo were isolated. Primary H2A.X −/− MEF cells were reconstituted with H2A.X constructs (WT or Y142F mutant), with or without an N-terminal FLAG epitope tag. The FLAG tag did not interfere with typical DNA damage response pathways, including γ-H2A.X. Since the expression of H2A.X at physiological levels in the reconstituted lines is important to study its function (Stucki et al., “MDC1 Directly Binds Phosphorylated Histone H2AX to Regulate Cellular Responses to DNA Double-Strand Breaks,” Cell 123:1213-1226 (2005), which is hereby incorporated by reference in its entirety), three independent cell lines (WT and mutants) were constructed in which the expression levels were comparable (50-80%) to the control primary MEFs (with two intact endogenous H2A.X alleles) and the majority of the reconstituted cells (˜90%) had similar expression levels. To enrich for proteins or complexes that may associate with H2A.X mononucleosomes, an approach that has previously been used to successfully purify such chromatin particles for subsequent biochemical purifications was adapted (Mendez et al, “Chromatin Association of Human Origin Recognition Complex, cdc6, and Minichromosome Maintenance Proteins During the Cell Cycle: Assembly of Prereplication Complexes in Late Mitosis,” Mol Cell Biol 20:8602-8612 (2000), which is hereby incorporated by reference in its entirety). This method, followed by immunoprecipitation with α-FLAG antibodies, significantly enriched H2A.X containing mononucleosomes and associated proteins (FIGS. 2A and 6). As shown by silver staining, a small number of polypeptides besides histones were associated with the wild type H2A.X mononucleosomes at a stoichiometric level, the amount of which was decreased after cells were treated with ionizing radiation (FIG. 2B). Subsequent mass spectrometry (MS) analysis demonstrated that two of the more prominent proteins are SNF2H (140 KDa), a mammalian homolog of the ISWI ATPase (Fyodorov et al., “The Many Faces of Chromatin Remodeling: SWItching Beyond Transcription,” Cell 106:523-525 (2001) and Becker et al., “ATP-Dependent Nucleosome Remodeling,” Annu Rev Biochem 71:247-273 (2002), which are hereby incorporated by reference in their entirety), and WSTF (William-Beuren syndrome Transcription Factor) (171 KDa), a.k.a. BAZ1B, a member of the BAZ/WAL family chromatin remodeling factors (FIG. 7) (Ito et al., “ACF Consists of Two Subunits, Acf1 and ISWI, That Function Cooperatively in the ATP-Dependent Catalysis of Chromatin Assembly,” Genes Dev 13:1529-1539 (1999); Poot et al., “HuCHRAC, a Human ISWI Chromatin Remodeling Complex Contains hACF1 and Two Novel Histone-Fold Proteins,” EMBO J. 19:3377-3387 (2000); Jones et al., “A Novel Family of Bromodomain Genes,” Genomics 63:40-45 (2000), which are hereby incorporated by reference in their entirety). These proteins constitute the WICH complex (WSTF-ISWI ATP-dependent chromatin-remodeling complex), which mobilizes nucleosomes in vitro and is suggested to be involved in the regulation of DNA replication (Poot et al., “The Williams Syndrome Transcription Factor Interacts with PCNA to Target Chromatin Remodeling by ISWI to Replication Foci,” Nat Cell Biol 6:1236-1244 (2004) and Bozhenok et al., “WSTF-ISWI Chromatin Remodeling Complex Targets Heterochromatic Replication Foci,” EMBO J. 21:2231-2241 (2002), which are hereby incorporated by reference in their entirety).

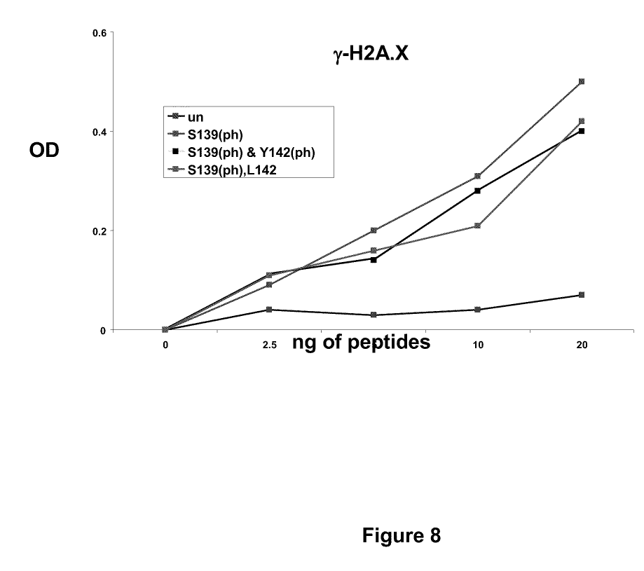

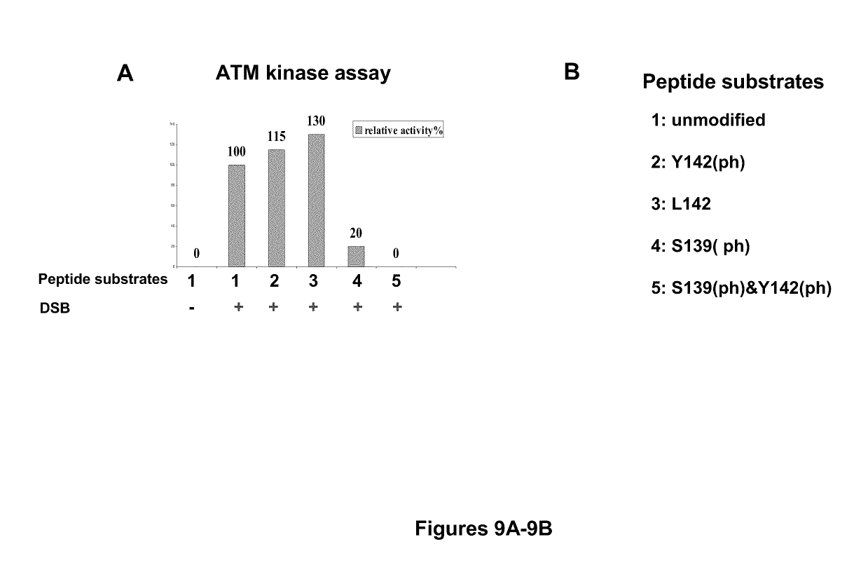

Whether other protein factors present in ISWI-containing chromatin remodeling complex, such as CHRAC15 and 17 (Kukimoto et al., “The Histone-Fold Protein Complex CHRAC-15/17 Enhances Nucleosome Sliding and Assembly Mediated by ACF,” Mol Cell 13:265-277 (2004), which is hereby incorporated by reference in its entirety), co-purified with H2A.X-containing mononucleosomes at sub-stoichiometric levels was investigated. However, none of these components have been identified by either MS or immunoblotting approaches, leading to the tentative conclusion that only the WICH complex co-purifies with H2A.X-containing mononucleosomes at significant levels in vivo. This association was then confirmed by immunoblot analyses of the complexes co-immunoprecipitated with H2A.X-containing nucleosomes. Consistent with the MS results, WSTF and SNF2H were enriched in these complexes (FIG. 2C). Interestingly, a significant decrease of γ-H2A.X phosphorylation levels in Y142F cells was observed (FIG. 2C and FIG. 4). Because of the close proximity between Ser139 and Tyr142 in the C-tail of H2A.X (FIG. 1A), the possibility that the mutation of Tyr142 or Tyr142 phosphorylation would influence Ser139 phosphorylation simply due to epitope disruption or impaired ATM/R kinase activity at Ser139 of H2A.X was investigated (See FIGS. 8 and 9). In addition, the interaction of the WICH complex is greatly reduced with Y142F mutant H2A.X mononucleosomes (FIGS. 2B and 2C). These results indicate that Y142 phosphorylation plays an important role in regulating H2A.X function during DNA damage.

To investigate the roles of WSTF in regulating the function of H2A.X, several independent WSTF knock-down NIH3T3 cell lines (WSTF RNAi cells) were generated by infection with a short hairpin WSTF RNAi construct, reducing WSTF protein expression significantly (FIG. 2D). Additionally, knockdown of SNF2H using a similar approach was attempted, but SNF2H knockdown resulted in S-phase defects and cell death as described in SNF2H knock-out mice studies (Stopka et al., “The ISWI ATPase Snf2h is Required for Early Mouse Development,” Proc Natl Acad Sci USA 100:14097-14102 (2003), which is hereby incorporated by reference in its entirety). Presumably, SNF2H functions in additional chromatin remodeling complexes that are involved in a variety of vital cell functions (Becker et al., “ATP-Dependent Nucleosome Remodeling,” Annu Rev Biochem 71:247-273 (2002), which is hereby incorporated by reference in its entirety). The specificity of the RNAi knock-down approach was demonstrated by rescuing WSTF expression with the human WSTF gene (the sequences in the WSTF mRNA targeted by the RNAi construct are different between human and mouse) (FIGS. 4E and 4F). Most interestingly, WSTF deficiency leads to a decrease in Y142 phosphorylation (FIG. 2D), indicating that WSTF may also be directly involved in regulating Y142 phosphorylation in vivo (see below). These data suggest a novel regulatory mechanisms of H2A.X involves WICH complex and Tyr142 phosphorylation.

### Example 14

### WSTF is a Novel Tyrosine Kinase that Phosphorylates Tyr142 of H2A.X

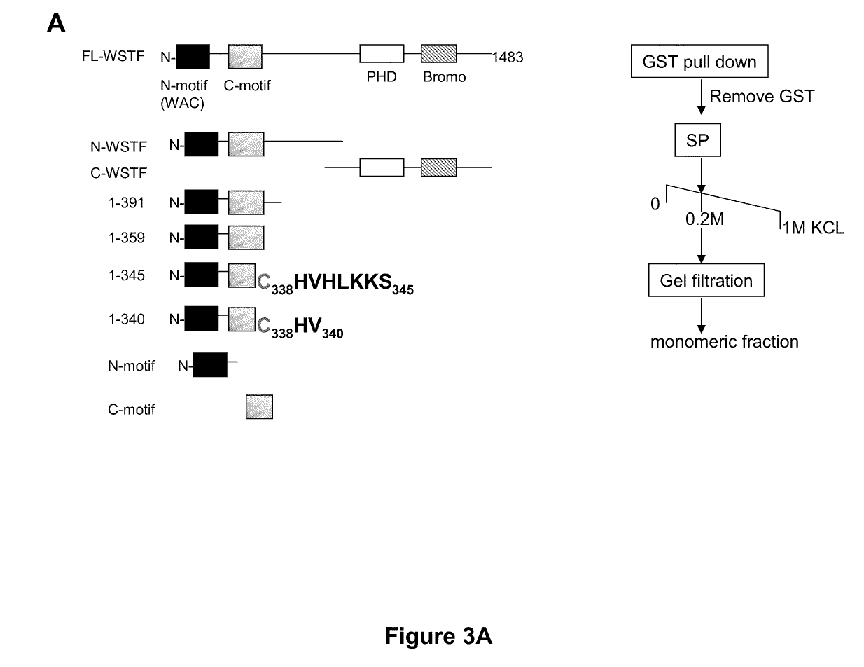

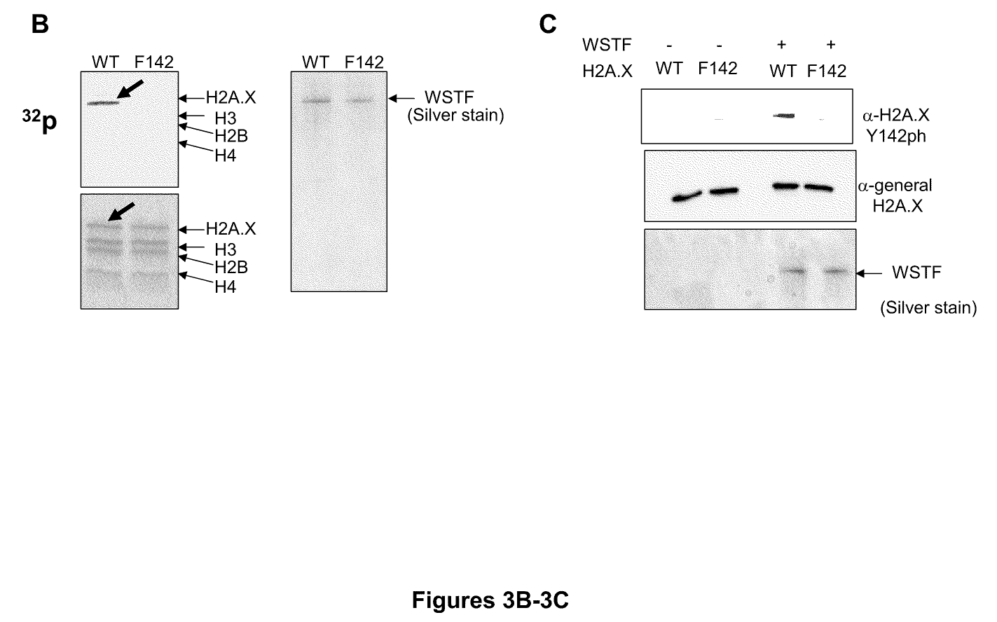

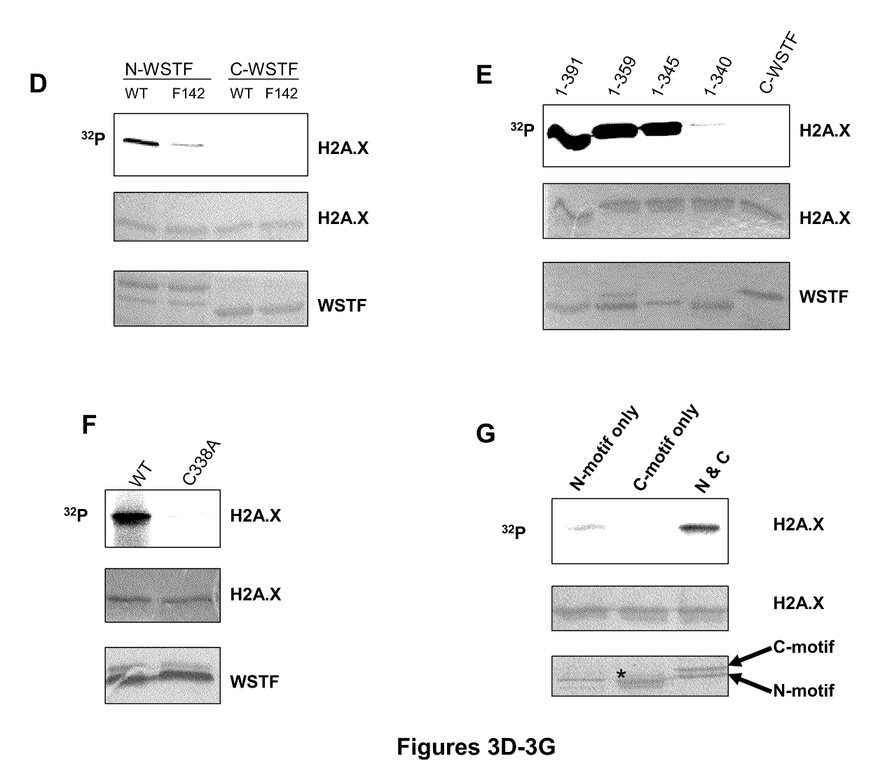

To further investigate the function of WSTF, purified recombinant, full-length human WSTF proteins were isolated from insect cells (FIG. 3A). A single protein band migrating at the expected molecular weight (171 KDa) was detected with silver staining (FIG. 3B). MS analysis on the purified samples confirmed that this protein was WSTF and did not detect any other proteins except trace amounts of heat shock proteins. The recombinant full length WSTF protein phosphorylated H2A.X-containing nucleosomes (FIG. 3B) that were reconstituted in vitro using ATP and divalent manganese ion Mn²⁺ (but not Mg²⁺, see FIG. 10A) as cofactors. To address whether WSTF can specifically phosphorylate Y142 of H2A.X, Y142F mutant nucleosomes were also reconstituted. The Y142F mutant does not interfere with histone octamer or nucleosome formation during reconstitution. In in vitro kinase assays, WSTF phosphorylated the WT but not Y142F H2A.X, indicating that Y142 is the major site of phosphorylation (FIG. 3B). The in vitro kinase activity of WSTF towards Y142 of H2A.X was confirmed by immunoblotting using the α-H2A.X Y142ph antibody (FIG. 3C).

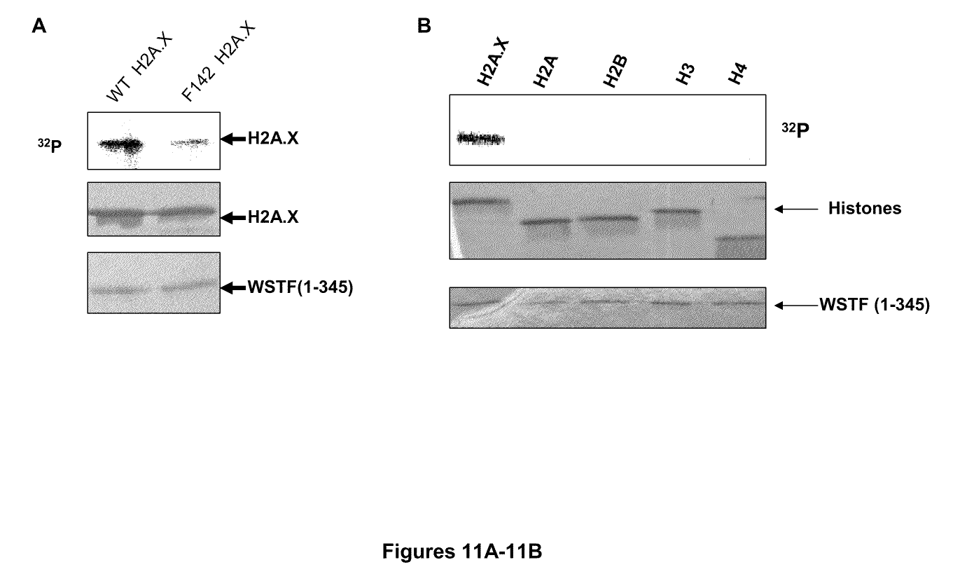

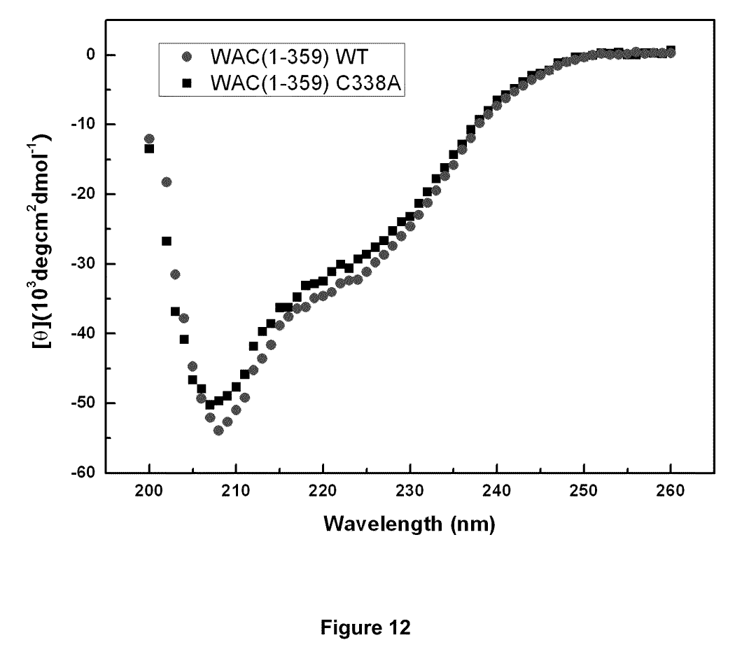

Since no detectable proteins other than trace amounts of heat shock proteins were co-purified with WSTF from insect cells (FIG. 3B), it was hypothesized that WSTF has an intrinsic kinase activity. However, WSTF does not share homology with known kinase domains. Therefore truncated recombinant WSTF proteins were generated (FIG. 3A) to investigate which portion of the protein is required for this novel kinase activity. Using this approach, it was determined that the N-terminal portion of WSTF is necessary and sufficient for kinase activity, strongly indicating that the kinase motif resides in its N-terminal portion (FIG. 3D). To map the putative kinase domain, a series of protein constructs within the N-terminal regions of WSTF were generated. All these protein constructs were extensively purified (FIG. 3A, right), and MS analysis did not identify any contaminating protein kinases. Strikingly, the 1-345 amino acid construct of WSTF had much more potent kinase activity (>50 fold) than the 1-340 construct (FIG. 3E). Furthermore, the substrate specificity of the 1-345 construct is similar to the full length WSTF (FIG. 11). These results suggest that the amino acid residues in the surrounding regions of 340-345 are critical for the kinase activity of WSTF. Consistent with these results, the activity of a point mutant at C338 (C338A) was greatly diminished (<50 fold, FIG. 3F). The CD spectrum of this mutant is identical to the WT counterpart (FIG. 12), indicating this mutation did not alter the global structure of the protein.

To further exclude the possibility that this activity might come from an undetected kinase co-purified at extremely low levels with WSTF (beyond the detection limit of silver stain and MS), recombinant WSTF proteins were generated in E coli since protein kinases, especially tyrosine kinases, are rare in the E. coli genome. The bioinformatical analysis of the kinase domain of WSTF reveals two well-structured motifs (N-motif, containing the conserved WAC domain (Jones et al., “A Novel Family of Bromodomain Genes,” Genomics 63:40-45 (2000), which is hereby incorporated by reference in its entirety) and a previously unreported C-motif, FIGS. 3A and 13). The polypeptides generated in the partial cleavage of the recombinant WSTF kinase domain also support this predication (FIG. 13). Based on these findings, recombinant protein constructs representing these two motifs from E. coli were generated. Co-expression of both motifs restores the kinase activity (which is perhaps related to the observation that these motifs are strongly associated) while the N-terminal motif alone had much reduced activity. These results strongly indicate that the N- and C-motif are required for the optimal kinase activity. Collectively, these results demonstrate that WSTF has a previously unidentified intrinsic kinase activity via a novel kinase domain.

### Example 15

### WSTF Plays a Critical Role in the DNA Damage Foci Formation

As the functions of the WICH complex, especially WSTF, in the DNA damage response have not been explored, the role of WSTF in this response was further investigated. Upon DNA damage treatment, the initial Ser139 phosphorylation of H2A.X and its foci formation in WSTF RNAi cells (as described in FIG. 2) were similar to control cells (up to 1-hour post IR, FIG. 4A). However, in control cells, the γ-H2A.X level remained unchanged until 16-hour post IR. In contrast, the level of γ-H2A.X phosphorylation rapidly declined in WSTF RNAi cells 4 hours after IR treatment (FIG. 4A). It is well-established that the number and morphology of γ-H2A.X foci undergo significant changes during the DNA damage response. Initially, a number of small foci are formed, while at late stage of the DNA damage response, only a few large foci are usually observed (Aten et al., “Dynamics of DNA Double-Strand Breaks Revealed by Clustering of Damaged Chromosome Domains,” Science 303:92-95 (2004), which is hereby incorporated by reference in its entirety). As expected, large γ-H2A.X foci were formed in control cells starting 4-hours post IR (FIG. 4B, upper panels) and persisting until 12-hours after IR, while the overall level of γ-H2A.X phosphorylation remains relatively constant. However, this morphological progression was not observed in WSTF RNAi cells despite the initial formation of small γ-H2A.X speckles as in the controls (FIG. 4B, lower panels). Instead, the amount and intensity of the γ-H2A.X foci were significantly less than in controls (only 16% of the WSTF RNAi retain γ-H2A.X foci 4 hours post IR), and no large foci were observed in WSTF RNAi cells during the entire DNA repair process, even after 12 hours post ionizing radiation (FIG. 4B). Since the ATM/R kinases are the major kinase for γ-H2A.X phosphorylation and γ-H2A.X foci maintenance is dependent on sustained recruitment of active ATM to the damage foci via Mdc1, (Stucki et al., “MDC1 Directly Binds Phosphorylated Histone H2AX to Regulate Cellular Responses to DNA Double-Strand Breaks,” Cell 123:1213-1226 (2005), which is hereby incorporated by reference in its entirety) whether the re-localization of ATM and Mdc1 is defective in the WSTF deficient cells was examined. ATM activation during the DNA damage response is reflected by its autophosphorylation of a serine 1981 (referred to as phos-ATM hereafter) (Bakkenist et al, “DNA Damage Activates ATM Through Intermolecular Autophosphorylation and Dimer Dissociation,” Nature 421:499-506 (2003), which is hereby incorporated by reference in its entirety), although the functional significance of this mark is unclear (Lee et al., “ATM Activation by DNA Double-Strand Breaks Through the Mre11-Rad50-Nbs1 Complex,” Science 308:551-554 (2005), which is hereby incorporated by reference in its entirety). In control cells, large phos-ATM foci were observed at late stage of DNA damage response (8-hour post 10 Gy of IR) (FIG. 4C), while similar foci were not observed in the WSTF deficient cells, indicating that the recruitment and/or the activation of ATM is defective at late stage of DNA damage response in these cells. Similarly, Mdc1foci formation was also greatly impaired in WSTF deficient cells (FIG. 4D). These data strongly suggest that WSTF plays a critical role in the recruitment of active ATM and Mdc1 to the damage sites, both of which are critical for γ-H2A.X foci formation.

Whether the kinase activity of WSTF is required for its function during the DNA damage response, namely, the maintenance of phos-ATM and γ-H2A.X foci, was next investigated. Since the WSTF mRNA sequences targeted by the short hairpin RNAi constructs are different between human and mouse (see above), WSTF RNAi cells (derived from mouse 3T3 cells) were complemented with wildtype or C338A mutant kinase domain constructs of human WSTF. This approach successfully restored the expression of kinase domain of WSTF (FIGS. 4E and 4F). The expression of the wildtype WSTF kinase domain rescued the γ-H2A.X and phos-ATM foci formation defects in the WSTF RNAi cells (FIGS. 4E and 4F). In nearly 100% of the cells complemented with the wildtype construct, γ-H2A.X and phos-ATM foci were observed at least 8 hours after DNA damage treatment (FIGS. 4E and F). The kinetics and morphology of these foci are indistinguishable from the control 3T3 cells. On the other hand, the phos-ATM and γ-H2A.X foci formation were as defective in cells expressing the C338A mutant as in WSTF RNAi cells containing vector alone (FIGS. 4E and F). These results demonstrate that WSTF has additional roles during the DNA damage response via its novel kinase activity in addition to regulating H2A.X Y142 phosphorylation.

To explore the in vivo role of Tyr142, whether the H2A.X Y142F and Y142L mutations would affect phosphorylation on Ser139 and foci formation were examined. γ-H2A.X levels and foci formation were greatly reduced upon DNA damage in both mutant cells (FIGS. 4G and 4H), despite similar expression levels of the wildtype and mutant H2A.X proteins. Because of the close proximity between Ser139 and Tyr142 in the C-terminus of H2A.X, the possibility that the mutation of Tyr142 or Tyr142 phosphorylation would influence Ser139 phosphorylation simply due to epitope disruption of the γ-H2A.X antibodies or impaired ATM/R kinase activity at Ser139 of H2A.X was investigated and ruled out (FIGS. 8 and 9A). These results are consistent with the DNA damage defects observed in the WSTF deficient cells. As recent studies have demonstrated that Tyr142 is critical for the interaction between Mdc1 and the H2A.X phosphorylated at Ser139 (Stewart et al., “MDC1 is a Mediator of the Mammalian DNA Damage Checkpoint,” Nature 421:961-966 (2003); Stucki et al., “MDC1 Directly Binds Phosphorylated Histone H2AX to Regulate Cellular Responses to DNA Double-Strand Breaks,” Cell 123:1213-1226 (2005); and Lee et al., “Structure of the BRCT Repeat Domain of MDC1 and its Specificity for the Free COOH-terminal End of the Gamma-H2AX Histone Tail,” J Biol Chem 280:32053-32056 (2005), which are hereby incorporated by reference in their entirety), whether mutations on Tyr142 would affect this interaction in vitro was tested. Only H2A.X peptides singly phosphorylated on Ser139 interacted with significant levels of Mdc1 from nuclear extracts, while phosphorylation or mutation of Tyr142 in these peptides greatly reduced this interaction (FIG. 4I). The recombinant Mdc1BRCT domain specifically bound to Ser139(ph) peptides, but not to unphosphorylated peptides and either phosphorylation or replacement of Tyr142 greatly reduced the binding (FIG. 4J), consistent with the results from nuclear extract pull-downs. Therefore, the observed γ-H2A.X defects in the Tyr142 mutant cells could be due to the impairment of Mdc1 binding to the damage sites. Taken together, these data suggest that WSTF plays a critical role in regulating DNA damage foci formation and H2A.X Tyr142 may have multiple functions during the DNA damage response.

### Discussion of Examples 1-15

These studies call attention to a new regulatory mechanism controlling histone H2A.X function mediated by the WSTF-SNF2H (WICH) chromatin remodeling complex. A novel phosphorylation site on H2A.X, Y142, was found that plays a vital role in the DNA damage response. It was also discovered that the amino-terminal domain of WSTF has a previously unrecognized tyrosine kinase activity for this site. WSTF, a gene frequently deleted in the human William Syndrome (WS), has previously been shown to be the regulatory component of the WICH chromatin remodeling complex (Ito et al., “ACF Consists of Two Subunits, Acf1 and ISWI, That Function Cooperatively in the ATP-Dependent Catalysis of Chromatin Assembly,” Genes Dev 13:1529-1539 (1999) and Bozhenok et al., “WSTF-ISWI Chromatin Remodeling Complex Targets Heterochromatic Replication Foci,” EMBO J. 21:2231-2241 (2002), which are hereby incorporated by reference in their entirety). The findings herein, therefore, have identified an unexpected link between a histone minor variant, H2A.X, a histone covalent modification, and an ATP-dependent chromatin remodeling mechanisms that control the mammalian DNA damage response.

The kinase domain of WSTF is composed of two putative motifs. The N-motif contains the highly conserved WAC domain, which is found in many eukaryotic proteins, while the C-motif is much more divergent. Structural and biochemical studies are underway to determine the molecular basis for the catalytic reaction of WSTF. Eukaryotic kinases often have several downstream targets and important factors in a signaling network may be modified by multiple kinases. Therefore, the outcome of a sophisticated signaling network can be controlled at multiple levels by crosstalk mechanisms (Hunter T., “Signaling—2000 and Beyond,” Cell 100:113-127 (2000), which is hereby incorporated by reference in its entirety). In light of this notion, WSTF may have other downstream targets given its multiple roles in DNA damage repair, replication, and transcription. William-Beuren syndrome is a well-documented genetic disorder characterized by developmental defects and clinical behavioral phenotypes (Francke U., “Williams-Beuren Syndrome: Genes and Mechanisms,” Hum Mol Genet 8:1947-1954 (1999), which is hereby incorporated by reference in its entirety), but the molecular mechanisms leading to the development of this disease is unclear. The link between the biochemical function of WSTF, including its intrinsic ability to bring about tyrosine phosphorylation of H2A.X and ensuing chromatin remodeling, and the clinical manifestation of WS remains a challenge for future studies.

The data demonstrate that WSTF also plays a critical role during the DNA damage response. In WSTF deficient cells, the recruitment of phos-ATM and Mdc1 to the DNA damage foci is compromised and the maintenance of γ-H2A.X foci is also defective. Since ATM and Mdc1 are critical for γ-H2A.X foci formation (Stucki et al., “MDC1 Directly Binds Phosphorylated Histone H2AX to Regulate Cellular Responses to DNA Double-Strand Breaks,” Cell 123:1213-1226 (2005), which is hereby incorporated by reference in its entirety) it is conceivable that observed γ-H2A.X foci maintenance defects in WSTF RNAi cells may be due to the defective phos-ATM and Mdc1 foci. The kinase activity of WSTF is also required for the foci formation of phos-ATM and in turn, γ-H2A.X, although the underlying mechanisms remain unclear. γ-H2A.X foci formation is defective in Y142 mutant cell lines. This result seems to suggest a critical role for Y142 phosphorylation in H2A.X DNA damage response. On the other hand, it was also found that mutation of Y142 to F or L reduces Mdc1 binding to H2A.X in vitro. Therefore, it is possible that H2A.X Y142F or Y142L mutants less efficiently recruit Mdc1 and ATM to the DNA damage sites, which causes γ-H2A.X foci formation defects.

A clue to how WSTF activity is regulated during the DNA damage response comes from the recent observation that the kinase domain of WSTF is hyper-phosphorylated in response to DNA damage, presumably by the ATM/R kinases (Matsuoka et al., “ATM and ATR Substrate Analysis Reveals Extensive Protein Networks Responsive to DNA Damage,” Science 316:1160-1166 (2007), which is hereby incorporated by reference in its entirety). This phosphorylation event could modulate the activity of WSTF and provide a mechanism to retain ATM at the damage foci. For example, phosphorylated WSTF may modify yet unknown histone or non-histone substrates, some, if not all, of which may facilitate the retention of ATM. Alternatively, phosphorylated WSTF, together with SNF2H, may remodel the chromatin structure to recruit and stimulate ATM activity as several studies have already implicated chromatin structure in the regulation of ATM activity (Bakkenist et al, “DNA Damage Activates ATM Through Intermolecular Autophosphorylation and Dimer Dissociation,” Nature 421:499-506 (2003) and You et al., “Rapid Activation of ATM on DNA Flanking Double-Strand Breaks,” Nat Cell Biol 9:1311-1318 (2007), which are hereby incorporated by reference in their entirety). In summary, although the underlying mechanism remains to be explored, WSTF plays a critical role in regulating the DNA damage response. The novel kinase activity may contribute to DNA damage foci formation via multiple pathways, some of which may be independent of H2A.X Y142.

A number of outstanding issues can be resolved to provide a more complete understanding of the H2A.X-mediated DNA repair pathway. First, the DNA damage induced dephosphorylation of Y142 coincides with S139 (γ-H2A.X foci) phosphorylation, which raises several interesting questions. For example, is Y142 dephosphorylation an obligatory step for γ-H2AX phosphorylation or does γ-H2A.X phosphorylation trigger a Y142 dephosphorylation in response to DNA damage? Identifying and characterizing the function of protein phosphatase(s) for H2A.X Y142 phosphorylation will provide valuable insights. Furthermore, are Ser139 and Y142 simultaneously phosphorylated on the same H2A.X molecule under certain physiological conditions? It is quite possible that the Y142-phosphorylated and doubly phosphorylated (Y142 and S139) H2A.X C-terminus, if indeed it exists in vivo, may recruit distinct, yet unknown binding effectors to mediate H2A.X function. Thus, identifying and characterizing these factors will be required to delineate the pathways connecting S139 and Y142 phosphorylation. Third, how Y142 phosphorylation levels are reduced in response to damage remains unclear. Are H2A.X nucleosomes that contain phosphorylated Y142 evicted from sites of damaged chromatin, or are these nucleosomes dephosphorylated in situ by a yet unknown phosphatase? Identification of signaling pathways utilizing H2A.X Tyr142 phosphorylation or other physiologically-relevant phosphorylation events regulated by WICH will be of great interest in future studies.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An antibody or antigen-binding fragment thereof that selectively binds to a phosphorylated tyrosine residue, wherein the phosphorylated tyrosine residue is located in an SQEY tyrosine phosphorylation motif sequence (SEQ ID NO: 2).

2. The antibody or antigen-binding fragment thereof according to claim 1, wherein the phosphorylated tyrosine residue of the SQEY motif is at amino acid position 142 of SEQ ID NO:1.

3. The antibody according to claim 1, wherein the antibody is monoclonal or polyclonal.

4. The antibody according to claim 1, wherein the antibody is multivalent or monovalent.

5. The antibody according to claim 1, wherein the antibody is humanized.

6. An antibody or antigen-binding fragment thereof that selectively binds to a phosphorylated serine residue, wherein the phosphorylated serine residue is closest to the glutamine residue in an KENSSQ phosphorylation motif sequence (SEQ ID NO:4).

7. The antibody or antigen-binding fragment thereof according to claim 6, wherein the phosphorylated serine residue of the KENSSQ motif is at amino acid position 167 of SEQ ID NO:3.

8. The antibody according to claim 6, wherein the antibody is monoclonal or polyclonal.

9. The antibody according to claim 6, wherein the antibody is multivalent or monovalent.

10. The antibody according to claim 6, wherein the antibody is humanized.

11. An isolated polypeptide molecule comprising an amino acid sequence of SEQ ID NO:6 or SEQ ID NO:7.

12. An isolated nucleic acid molecule encoding the polypeptide of claim 11.

13. The isolated nucleic acid molecule of claim 12, wherein the nucleic acid molecule has a nucleotide sequence that has at least 80% nucleic acid sequence identity to the nucleotide sequence of SEQ ID NO: 5.

14. The isolated nucleic acid molecule of claim 12, wherein the nucleic acid molecule has a nucleotide sequence that has at least 90% nucleic acid sequence identity to the nucleotide sequence of SEQ ID NO:5.

15. The isolated nucleic acid molecule of claim 12, wherein the nucleic acid molecule has a nucleotide sequence that has at least 95% nucleic acid sequence identity to the nucleotide sequence of SEQ ID NO:5.

16. An expression vector comprising the isolated nucleic acid molecule of claim 12.

17. A host cell containing the expression vector of claim 16.

18. A method of identifying cellular DNA damage in a sample, said method comprising:
providing a cell sample;
detecting the presence or absence of a phosphorylated tyrosine residue in an SQEY tyrosine phosphorylation motif (SEQ ID NO:2); and
identifying cellular DNA damage in the cell sample based on said detecting.

19. The method according to claim 18, wherein the phosphorylated tyrosine residue of the SQEY motif is at amino acid position 142 of SEQ ID NO:1.

20. The method according to claim 18, wherein said detecting comprises contacting the sample with an antibody or antigen-binding fragment thereof that selectively binds to the phosphorylated tyrosine residue in an SQEY tyrosine phosphorylation motif (SEQ ID NO:2).

21. The method according to claim 20, wherein said antibody or antigen-binding fragment thereof selectively binds to the phosphorylated tyrosine residue of the SQEY motif at amino acid position 142 of SEQ ID NO:1.

22. The method according to claim 18, wherein the absence of a phosphorylated tyrosine residue in the SQEY motif indicates the presence of cellular DNA damage.

23. The method according to claim 18, wherein the presence of a phosphorylated tyrosine residue in the SQEY motif indicates the absence of cellular DNA damage.

24. A method of identifying cellular DNA damage in a sample, said method comprising:
providing a cell sample;
detecting the presence or absence of a phosphorylated serine residue at the serine closest to the glutamine residue in an KENSSQ phosphorylation motif sequence (SEQ ID NO:4); and
identifying cellular DNA damage in the cell sample based on said detecting.

25. The method according to claim 24, wherein the phosphorylated serine closest to the glutamine residue of the KENSSQ motif is at amino acid position 167 of SEQ ID NO:3.

26. The method according to claim 24, wherein said detecting comprises contacting the sample with an antibody or antigen-binding fragment thereof that selectively binds to the phosphorylated serine closest to the glutamine residue in an KENSSQ phosphorylation motif sequence (SEQ ID NO:4).

27. The method according to claim 26, wherein said antibody or antigen-binding fragment thereof selectively binds to the phosphorylated serine closest to the glutamine residue in the KENSSQ motif at amino acid position 167 of SEQ ID NO:3

28. The method according to claim 24, wherein the presence of a phosphorylated serine residue at the serine closest to the glutamine residue of the KENSSQ motif indicates the presence of cellular DNA damage.

29. The method according to claim 24, wherein the absence of a phosphorylated serine residue at the serine closest to the glutamine residue of the KENSSQ motif indicates the absence of cellular DNA damage.

30. A method of screening candidate compounds useful for modulating WSTF kinase activity, said method comprising:
providing a candidate compound;
providing a sample, wherein said sample comprises a WSTF protein or polypeptide having kinase activity, a protein substrate having an SQEY motif sequence, manganese, and ATP;
contacting the candidate compound with the sample;
detecting the presence or absence of a phosphorylated tyrosine residue in the SQEY tyrosine phosphorylation motif (SEQ ID NO:2); and
identifying compounds useful for modulating WSTF kinase activity based on said detecting.

31. The method according to claim 30, wherein the WSTF protein or polypeptide having kinase activity comprises SEQ ID NO: 6 or SEQ ID NO:7.

32. The method according to claim 30, wherein the phosphorylated tyrosine residue in the SQEY motif is at amino acid position 142 of SEQ ID NO:1 in the sample.

33. The method according to claim 30, wherein the sample is a cell sample.

34. The method according to claim 30, wherein detecting the absence of a phosphorylated tyrosine residue in the SQEY motif identifies a compound useful for inhibiting WSTF kinase activity.

35. A method of screening candidate compounds useful for modulating cellular DNA damage repair, said method comprising;
providing a candidate compound;
providing a cell sample;
contacting the candidate compound with the cell sample;
detecting the presence or absence of a phosphorylated tyrosine residue in a SQEY tyrosine phosphorylation motif (SEQ ID NO:2) in the cell sample; and
identifying candidate compounds useful for modulating cellular DNA damage repair based on said detecting.

36. The method according to claim 35, wherein the phosphorylated tyrosine residue in the SQEY motif is at amino acid position 142 of SEQ ID NO:1 in the cell sample.

37. The method according to claim 35, wherein the cell sample comprises one or more genetically modified yeast cells, wherein the genetically modified yeast cells contain a leucine to tyrosine mutation in their H2A.X SQEL motif.

38. The method according to claim 35, wherein said contacting occurs in the presence of a DNA damaging agent.

39. A method of screening candidate compounds useful for treating cancer, said method comprising;
providing a candidate compound;
providing a cell sample;
contacting the candidate compound with the cell sample;
detecting the presence or absence of a phosphorylated tyrosine residue in an SQEY tyrosine phosphorylation motif (SEQ ID NO:2) in the cell sample; and
identifying candidate compounds useful for treating cancer based on said detecting.

40. The method according to claim 39, wherein the phosphorylated tyrosine residue in the SQEY motif is at amino acid position 142 of SEQ ID NO:1 in the cell sample.

41. The method according to claim 39, wherein the cell sample comprises one or more genetically modified yeast cells, wherein the genetically modified yeast cells contain a leucine to tyrosine mutation in their H2A.X SQEL motif.

42. A method of modulating cellular DNA damage repair, said method comprising:
administering to a cell an agent which modulates tyrosine phosphorylation of an SQEY tyrosine phosphorylation motif (SEQ ID NO:2) under conditions effective to modulate cellular DNA damage repair.

43. The method according to claim 42, wherein said agent modulates tyrosine phosphorylation of the tyrosine residue in the SQEY motif at amino acid position 142 of SEQ ID NO:1 in the cell.

44. The method according to claim 42, wherein an agent that reduces tyrosine phosphorylation of the SQEY motif enhances cellular DNA damage repair.

45. The method according to claim 44, wherein the agent is a WSTF kinase domain inhibitor.

46. The method according to claim 45, wherein the WSTF kinase domain inhibitor is an siRNA molecule comprising SEQ ID NO: 21.

47. The method according to claim 44, wherein the agent is a recombinant EYA phosphatase protein.

48. The method according to claim 42, wherein an agent that induces tyrosine phosphorylation of the SQEY motif suppresses cellular DNA damage repair.

49. The method according to claim 48, wherein the agent is a nucleic acid molecule encoding a WSTF kinase domain comprising the nucleotide sequence of SEQ ID NO: 5 or the encoded polypeptide comprising an amino acid sequence of SEQ ID NO: 6 or SEQ ID NO:7.

50. The method according to claim 48, wherein the agent is an EYA phosphatase protein inhibitor.

51. A method of treating a subject having cancer, said method comprising:
administering to said subject having cancer, an agent that modulates tyrosine phosphorylation of a SQEY tyrosine phosphorylation motif (SEQ ID NO:2) under conditions effective to treat the subject having cancer.

52. The method according to claim 51, wherein said agent modulates tyrosine phosphorylation of the tyrosine residue in the SQEY motif at amino acid position 142 of SEQ ID NO:1.

53. The method according to claim 51, wherein the agent reduces tyrosine phosphorylation of the SQEY motif.

54. The method according to claim 53, wherein the agent is a WSTF kinase domain inhibitor.

55. The method according to claim 54, wherein the WSTF kinase domain inhibitor is an siRNA molecule comprising SEQ ID NO: 21.

56. The method according to claim 53, wherein the agent is a recombinant EYA phosphatase protein.

57. The method according to claim 51, wherein the agent induces tyrosine phosphorylation of the SQEY motif.

58. The method according to claim 57, wherein the agent is a nucleic acid molecule encoding a WSTF kinase domain comprising the nucleotide sequence of SEQ ID NO: 5 or the encoded polypeptide comprising an amino acid sequence of SEQ ID NO: 6 or SEQ ID NO:7.

59. The method according to claim 57, wherein the agent is an EYA phosphatase protein inhibitor.
